(19) United States
(12) Patent Application Publication
(10) Pub. No.: US 2008/0234183 A1
(43) Pub. Date: Sep. 25, 2008

(54) Cell Penetrating Peptides

(76) Inventors: Mattias Hallbrink, Stockholm (SE); Margus Pooga, Tartu (EE); Madis Metsis, Solna (SE); Priit Kogerman, Tabasalu (EE); Andreas Valkna, Viimsi (EE); Anne Meikas, Saku Estonia (EE); Maria Lindgren, Stockholm (SE); Astrid Graslund, Sollentuna (SE); Goran Eriksson, Stockholm (SE); Claes Goran Ostensson, Solna (SE); Metka Budihna, Ljubljana-Sentvid (SI); Matjaz Zorko, Skoffjica (SI); Anna Elmquist, Norrtalje (SE); Ursel Soomets, Tartu (EE); Pontus Lundberg, Solna (SE); Peter Jarver, Stockholm (SE); Kulliki Saar, Stockholm (SE); Samir El-Andaloussi, Stockholm (SE); Kalle Kilk, Danderyd (SE); Ulo Langel, Bandhagen (SE)

Correspondence Address: Andrew K Gonsalves;Nixon Peabody, Clinton Square, PO Box 31051, Rochester, NY, 14603-1051, US

(21) Appl. No.: 10/517,079

(22) PCT Filed: Jun. 18, 2003
(86) PCT No.: PCT/IB03/03163

§ 371 Date: Aug. 11, 2005

Related U.S. Application Data
(60) Provisional application No. 60/391,788, filed on Jun. 25, 2002.

(30) Foreign Application Priority Data
Jun. 18, 2002 (SE) 0201863-8

(51) Int. Cl. A61K 38/16; C12Q 1/02; C07K 7/06; C07K 7/08; C07K 14/00; A61K 31/7052; A61P 25/00; A61P 35/00; A61P 9/00; A61P 31/00; A61P 3/10; C12N 9/00; C12N 15/63; A61K 38/08; A61K 38/10

(52) U.S. Cl. 514/12; 435/29; 530/324; 530/325; 530/326; 530/327; 530/328; 435/183; 435/320.1; 514/13; 514/14; 514/15; 514/16; 514/44

(57) ABSTRACT

The present invention relates to a method for predicting or designing, detecting, and/or verifying a novel cell-penetrating peptide (CPP) and to a method for using said new CPP and/or a novel usage of a known CPP for an improved cellular uptake of a cellular effector, coupled to said CPP. Furthermore, the present invention also relates to a method for predicting or designing, detecting and/or verifying a novel cell-penetrating peptide (CPP) that mimics cellular effector activity and/or inhibits cellular effector activity. The present invention additionally relates to the use of said CPP for treating and/or preventing a medical condition and to the use of said CPP for the manufacture of a pharmaceutical composition for treating a medical condition.

### FIELD OF THE INVENTION

The present invention relates to a method for predicting, designing, detecting, and/or verifying a novel cell-penetrating peptide (CPP) and to a method for using said new CPP and/or a novel usage of a known CPP for an improved cellular uptake of a cellular effector, coupled to said CPP. Furthermore, the present invention also relates to a method for predicting, designing, detecting and/or verifying a novel cell-penetrating peptide (CPP) that mimics cellular effector activity and/or Inhibits cellular effector activity. The present invention additionally relates to the use of any of said CPP for treating and/or preventing a medical condition and to the use of any of said CPP for the manufacture of a pharmaceutical composition for treating a medical condition.

### BACKGROUND OF THE INVENTION

A number of techniques have been developed to deliver different cellular effectors into cells. The majority of these techniques are invasive, like electroporation or microinjection. Liposome encapsulation and receptor-mediated endocytosis are milder methods, but they unfortunately suffer from serious drawbacks, in particular, low delivery yield.

The established view in cellular biology dictates that the cellular Internalsation of hydrophilic macromolecules can only be achieved through the classical endocytosis pathway. However, in the last decade, several peptides have been demonstrated to translocate across the plasma membrane of eukaryotic cells by a seemingly energy-independent pathway. These peptides are defined as cell-penetrating peptides (CPPs) and have been used successfully for intracellular delivery of macromolecules with molecular weights several times greater than their own. (M. Undgren et al, 2000, Cell-penetrating peptides; TIPS, Vol. 21, pg. 99-103)

Cellular delivery using these cell-penetrating peptides offers several advantages over conventional techniques. It is non-invasive, energy-independent, is efficient for a broad range of cell types and can be applied to cells en masse. Furthermore, it has been found that for certain types of CPPs, cellular internalisation occurs at 37° C., as well as at 4° C. and that it can not be saturated. Also, in most cases, the Internalisation seems not to require a chiral receptor protein, since no enantiomeric discrimination has been observed.

Until recently, transport of hydrophilic macromolecules into the cytoplasmic and nuclear compartments of living cells without disrupting the plasma membrane seemed a far-off goal. Because of their low biomembrane permeability and their relatively rapid degradation, polypeptides and oligonucleotides were generally considered to be of limited therapeutic value. This is an obstacle in both biomedical research and the pharmaceutical industry.

An even more difficult, although very important task, is to deliver hydrophilic macromolecules across the blood-brain barrier. Several methods have been envisaged to overcome this hurdle. Nevertheless, they all suffer from limitations, such as their effectiveness being restricted to a subset of molecules, or that they give a too low yield. However, recent reports suggest that CPPs might be able to transport macromolecules across the blood-brain barrier.

Another essential area for desired delivery of effectors is nuclear import, wherein, in general, it has been found that the signal sequence must contain some positively charged (basic) residues (Moroianu J., J. Cell Biochem, 1999). It seems that such charged amino acids might also be required for plasma membrane translocation.

Today, a diversity of cell-penetrating peptides, CPPs, is known. Several peptides have been demonstrated to translocate across the plasma membrane of eukaryotic cells by a seemingly energy-independent pathway. Thus, cell-penetrating peptides might be used as delivery vectors for pharmacologically interesting substances, such as peptides, proteins, oligonucleotides, antisense molecules, as well as for research tools.

Of particular interest among CPPs are those peptides that have low lytic activity. These translocating peptides, also known as Trojan peptides (D. Derossi et al., Trends Cell Biol. 8 (1998) 84-87), have been applied as vectors for the delivery of hydrophilic biomolecules and drugs into cytoplasmic and nuclear compartments of cells, both In vivo and in vitro (for review, see M. Lindgren et al., Trends Pharmacol. Sci. 21 (2000) 99-103). When covalently linked with a cargo, including polypeptides and oligonucleotides with many times their own molecular mass, these peptides are still able to translocate.

Examples of useful transport peptides are sequences derived from homeodomains of certain transcription factors, as well as so-called Tat-derived peptides and peptides based on signal sequences. The first of the homeodomain-derived translocating peptides was penetratin, denoted pAntp, with a sequence corresponding to the 16 residues of the third α-helix (residues 43-58) from the Antennapedia homeodomain protein of Drosophila (D. Derossi et al., J. Biol. Chem. 269 (1994) 10444-10450; A. Prochiantz, Ann. NY Acad. Sci. 886 (1999) 172-179). The pAntp peptide retains its membrane translocation properties and has therefore been proposed to be a universal Intercellular delivery vector (D. Derossi et al., Trends Cell Biol. 8 (1998) 84-87).

Purely synthetic or chimeric peptides have also been designed, as reviewed in (D. Derossi et al., Trends Cell Biol. 8 (1998) 84-87, and M. Lindgren et al., Trends Pharmacol. Sci. 21 (2000) 99-103).

Transportan, e.g., a non-natural peptide, is able to deliver an antibody molecule with a molecular mass of about 150 kDa over the plasma membrane, although Transportan itself is only a 3 kDa peptide. Transportan and penetratin were demonstrated to deliver a non-natural DNA analogue, PNA (peptide nucleic acid) into cytoplasm and nuclei of cells in culture (Pooga et al. 1998, Nature Biotech.).

Another group of peptides that have surprisingly been shown to be able to transport across the cellular membrane, when coupled to a hydrophobic moiety, are modified receptors, in particular G protein coupled receptors, which are called pepducines (see e.g. WO0181408, Kuliopulos, et. al.). It was discovered that attachment of a hydrophobic moiety to peptides derived from the third intracellular loop of a 7™ receptor yields cellular translocation of said chimeric peptides and full agonist and/or antagonist of receptor G-protein signalling. These pepducines are membrane Inserting, membrane-tethered chimeric peptides and require the presence of their cognate receptor for activity and are highly selective for receptor type.

Although their astonishing transport capability has put CPPs into the focus of scientific interest for the last years, the most basic mechanisms of translocation for the different CPPs is still unknown. For instance, it is today still not known in the field, whether any particular secondary structure has to be induced in order to allow (energetically) a translocation, involving a concomitant transient membrane destabilization. It is clear, however, that the molecular details of the peptide-membrane interactions must be of fundamental importance for the translocation process.

The mechanism and requirements for internalisation have been studied on interactions between amphipathic α-helical peptides and lipid (bl)layers. The results of these studies often suggest tryptophan to be responsible for internalisation of a peptide, but although aromatic amino acids may be preferred in CPP sequences, they are not absolutely necessary for cell penetration.

Apart from the cell penetration capability, little correlation of structure or behaviour has been found between CPPs. Up to now, CPPs have thus not been designed in a rational manner, but have been found serendipitously. However, the sequences of CPPs published so far have a positive net-charge as the only common feature, giving a starting point for the prediction of CPP functionality in a given peptide sequence. Clearly, though, all sequences with a positive net-charge cannot be cell-penetrating, Indicating that further restrictions are needed to select CPPs with any certainty.

The present invention for the first time provides a novel general principle for predicting, designing, detecting and/or verifying a cell-penetrating peptide and/or a non-peptide analogue thereof, characterised by application of an assortment of novel prediction/selection criteria, optionally in combination with a method for testing the cellular penetration capacity of said found CPP in vitro and/or in vivo.

The present invention not only facilitates the much more effective and precise selection of CPP-active naturally occurring peptide fragments, that could maybe, but without doubt much more laboriously, have been found through trial and error, but also for the first time makes it at all possible to design such a desired CPP de novo. What is more, the present invention for the first time makes it possible to modify a correctly predicted naturally occurring CPP to improve its cell-penetrating effectiveness or to suit a secondary specific need, without loosing its cell-penetrating ability.

The present invention relates to a method for predicting, detecting, designing and/or verifying a cell-penetrating peptide (CPP) and/or a non-peptide analogue thereof, characterised by application of novel prediction/selection criteria, optionally in combination with a method for testing the cellular penetration capacity of said found CPP in vitro and/or in vivo.

A unifying aspect of the invention is thus directed to a method of identifying a cell-penetrating amino acid fragment, comprising assessing the bulk property value Z_{Σ} of said sequence, Z_{Σ} comprising at least 5 individual average interval values Z_{Σ1}; Z_{Σ2}; Z_{Σ3}; Z_{Σ4} and Z_{Σ5}, wherein Z_{Σ1}, Z_{Σ2}, Z_{Σ3}, Z_{Σ4} and Z_{Σ5} are average values of the respective descriptor values for the residues in said amino acid sequence, calculated with the formula

Z_{Σx}=(Zₓᵣₑₛ₁+Zₓᵣₑₛ₂ . . . +Zₓᵣₑₛₙ)/n

Z_{xresy} being the respective descriptor value for amino acid residue y comprised in the selected candidate fragment, and wherein the descriptor value of each residue corresponds to a Z₁, Z₂, Z₃, Z₄, and Z₅ descriptor value in a descriptor value scale, and wherein a cell-penetrating fragment is characterised by having a Z_{Σ} bulk property value essentially consisting of Individual average interval values, wherein Z_{Σ1}<0.2; Z_{Σ2}<1.1; Z_{Σ3}<−0.49; Z_{Σ4}<0.33; and Z_{Σ5}5<1.1 and Z_{Σ5}>0.12.

In a presently preferred embodiment of the invention, the above selection criteria are furthermore supplemented by a three graded system for successive narrowing of the descriptor interval, wherein two additional descriptors are introduced: Bulkₕₐ being the number of non-hydrogen atoms (e.g. C, N, S and O) in the side chains of the amino acids, and hdb standing for the number of accepting hydrogen bonds for the side chains of the amino acids.

The invention further relates to a method for using said novel CPP and/or to a novel and improved usage of a known CPP for improved cellular uptake of a cellular effector coupled to said CPP, and to a method for predicting, detecting, designing and/or verifying a novel cell-penetrating peptide (CPP) that has cellular effector activity itself. Furthermore, the present invention also relates to the use of said CPP and/or said improved usage of a known CPP for treating and/or preventing a medical condition, and/or for the manufacture of a pharmaceutical composition for treating a medical condition.

### DETAILED DISCLOSURE

The present invention for the first time discloses a novel general principle for predicting, designing, detecting and/or verifying a cell-penetrating peptide and/or a non-peptide analogue thereof, characterised by application of a novel prediction/selection criterion, optionally in combination with a method for in vivo and/or in vitro testing the cellular penetration capacity of said found CPP, either derived from a random de novo sequence or a naturally occurring protein, or a non-peptide analogue thereof.

### Evaluation of Predictors Relevant for the Function of Cell-Penetrating Peptides

In most peptide quantitative structure activity relationship studies (QSAR), a set of dimensionless values is used to describe a composite of the physical characteristics of the amino acids. In the classical literature, 3 values, Z₁, Z₂ and Z₃ are used for this purpose. Recently, Wold and colleagues expanded this descriptor set with 2 more: Z₄ and Z₅; and produced descriptor scales covering 87 natural and non-natural amino acids (Sandberg, M., Eriksson, L., Jonsson, J., Sjastrom, M., and Wold, S., New chemical descriptors relevant for the design of biologically active peptides. A multivariate characterization of 87 amino acids, J. Med. Chem., 41, 2481 (1998).

The novel methods described in the present application comprise using said expanded QSAR descriptor scales for the evaluation of CPP functionality in any given naturally occurring peptide or for de novo designing a CPP. Said new method thus opens a fast and reliable way to the production of CPPs, consisting of natural as well as non-natural building blocks. Moreover, a rigorous quantification of CPP uptake in a variety of physical tests as disclosed herein, even enables a QSAR model for tissue specificity.

TABLE 1A
Descriptor Scales for the
Characterized Coded and Non-Coded Amino Acids

| no. | abbrev | name^{a} | z₁ | z₂ | z₃ | z₄ | z₅ |
|---|---|---|---|---|---|---|---|
| 1 | Ala | alanine | 0.24 | −2.32 | 0.60 | −0.14 | 1.30 |
| 2 | Arg | arginine | 3.52 | 2.50 | −3.50 | 1.99 | −0.17 |
| 3 | Asn | asparagine | 3.05 | 1.62 | 1.04 | −1.15 | 1.61 |
| 4 | Asp | aspartic acid | 3.98 | 0.93 | 1.93 | −2.46 | 0.75 |
| 5 | Cys | cysteine | 0.84 | −1.67 | 3.71 | 0.18 | −2.65 |
| 6 | Gln | glutamine | 1.75 | 0.50 | −1.44 | −1.34 | 0.66 |
| 7 | Glu | glutamic acid | 3.11 | 0.26 | −0.11 | −3.04 | −0.25 |
| 8 | Gly | glycine | 2.05 | −4.06 | 0.36 | −0.82 | −0.38 |
| 9 | His | histidine | 2.47 | 1.95 | 0.26 | 3.90 | 0.09 |
| 10 | Ile | isoleucine | −3.89 | −1.73 | −1.71 | −0.84 | 0.26 |
| 11 | Leu | leucine | −4.28 | −1.30 | −1.49 | −0.72 | 0.84 |
| 12 | Lys | lysine | 2.29 | 0.89 | −2.49 | 1.49 | 0.31 |
| 13 | Met | methionine | −2.85 | −0.22 | 0.47 | 1.94 | −0.98 |
| 14 | Phe | Phenyl- | −4.22 | 1.94 | 1.06 | 0.54 | −0.62 |
| | | alanine | | | | | |
| 15 | Pro | proline | −1.66 | 0.27 | 1.84 | 0.70 | 2.00 |
| 16 | Ser | serine | 2.39 | −1.07 | 1.15 | −1.39 | 0.67 |
| 17 | Thr | threonine | 0.75 | −2.18 | −1.12 | −1.46 | −0.40 |
| 18 | Trp | tryptophan | −4.36 | 3.94 | 0.59 | 3.44 | −1.59 |
| 19 | Tyr | tyrosine | −2.54 | 2.44 | 0.43 | 0.04 | −1.47 |
| 20 | Val | valine | −2.59 | −2.64 | −1.54 | −0.85 | −0.02 |
| 21 | Acpa | Aminocaprylic | −4.38 | 1.92 | 2.14 | −2.61 | −4.93 |
| | | acid | | | | | |
| 22 | Aecys | (S)-2- | 3.03 | 2.60 | 0.50 | 2.65 | −1.55 |
| | | aminoethyl-L- | | | | | |
| | | cysteine•HCl | | | | | |
| 23 | Afa | aminophenylacetate | −3.51 | 2.93 | 2.94 | 1.17 | 1.22 |
| 24 | Aiba | -aminoiso- | −1.33 | −2.80 | −0.61 | −0.55 | 0.40 |
| | | bytyric acid | | | | | |
| 25 | Aile | alloisoleucine | −4.09 | −1.28 | −1.40 | −0.63 | 0.94 |
| 26 | Alg | L-allylglycine | −2.31 | −1.35 | −0.05 | 0.05 | 1.25 |
| 27 | Aba | aminobutyric | −1.22 | −2.44 | −0.38 | −0.51 | 0.65 |
| | | acid | | | | | |
| 28 | Aphe | p- | −0.62 | 3.28 | −0.11 | 3.24 | −1.51 |
| | | aminophenylalanine | | | | | |
| 29 | Bal | -alanine | 2.16 | −6.54 | −4.46 | −2.66 | −5.93 |
| 30 | Brphe | p- | −5.62 | 3.18 | 0.29 | 0.54 | −1.10 |
| | | bromophenylalanine | | | | | |
| 31 | Cha | cyclohexylalanine | −6.26 | 0.30 | −2.58 | −0.67 | 1.01 |
| 32 | Cit | citrulline | 1.31 | 1.47 | −2.76 | −2.10 | 0.42 |
| 33 | Clala | -chloroalanine | −0.66 | 0.30 | 2.65 | −0.47 | 1.92 |
| 34 | Cle | cycloleucine | −2.95 | −2.16 | −1.66 | −0.65 | 0.19 |
| 35 | Clphe | p- | −5.31 | 2.66 | 0.99 | 0.02 | −1.76 |
| | | chlorophenylalanine | | | | | |
| 36 | Cya | cysteic acid | 4.20 | 3.59 | 3.76 | −5.09 | −1.36 |
| 37 | Dab | 2,4-diamino- | 3.69 | −0.53 | −0.24 | 1.03 | −0.15 |
| | | butyric acid | | | | | |
| 38 | Dap | 2,3- | 4.34 | −0.54 | 0.96 | 1.04 | 0.24 |
| | | diaminopropionic | | | | | |
| | | acid | | | | | |
| 39 | Dhp | 3,4-dehydro- | −1.24 | 0.40 | 2.50 | 1.48 | 1.53 |
| | | proline | | | | | |
| 40 | Dhphe | 3,4- | −0.45 | 3.32 | −0.07 | −0.33 | −1.95 |
| | | dihydroxy- | | | | | |
| | | phenyl- | | | | | |
| | | alanine | | | | | |
| 41 | Fphe | p- | −4.58 | 2.26 | 1.28 | −0.70 | −1.58 |
| | | fluorophenylalanine | | | | | |
| 42 | Gaa | D-glucose- | 4.90 | 3.91 | −1.98 | −4.18 | 0.89 |
| | | aminic acid | | | | | |
| 43 | Hag | Homo- | 2.70 | 3.06 | −4.15 | 2.32 | −0.46 |
| | | arginine | | | | | |
| 44 | Hlys | hydroxyl- | 3.98 | 1.67 | −2.51 | 0.32 | 0.08 |
| | | lysine•HCl | | | | | |
| 45 | Hnvl | DL-hydroxynorvaline | −0.85 | −1.08 | −1.10 | −1.73 | −0.04 |
| 46 | Hog | Homoglutamine | 1.33 | 1.19 | −2.14 | −1.61 | 0.59 |
| 47 | Hoph | homophenylalanine | −5.86 | 2.95 | 0.37 | 1.03 | 0.32 |
| 48 | Hos | homoserine | 0.93 | −0.71 | −0.01 | −1.58 | 0.94 |
| 49 | Hpr | hydroxyl- | −0.24 | 2.27 | 2.47 | 0.18 | 2.94 |
| | | proiine | | | | | |
| 50 | Iphe | p-iodophenylalanine | −6.23 | 6.88 | 3.01 | 1.52 | 1.05 |
| 51 | Ise | isoserine | 3.78 | 2.82 | 2.55 | 0.27 | 2.96 |
| 52 | Mle | -methyl- | −5.40 | −2.07 | −2.86 | −1.15 | −0.27 |
| | | leucine | | | | | |
| 53 | Msmet | DL- | 1.22 | 1.89 | −0.91 | 3.75 | −1.25 |
| | | methionine-s-methyl- | | | | | |
| | | sulfoniumchloride | | | | | |
| 54 | 1Nala | 3-(1- | −5.67 | 6.31 | 3.43 | 3.51 | −0.47 |
| | | naphthyl)alanine | | | | | |
| 55 | 2Nala | 3-(2- | −6.48 | 6.37 | 2.81 | 3.02 | −0.49 |
| | | naphthyl)alanine | | | | | |
| 56 | Nle | norleucine (or | −4.33 | −1.30 | −1.54 | −0.85 | 0.74 |
| | | 2-aminohexanoic | | | | | |
| | | acid) | | | | | |
| 57 | Nmala | N-methyl- | −1.30 | −3.13 | −0.65 | 0.04 | −0.16 |
| | | alanine | | | | | |
| 58 | Nva | norvaline (or | −3.08 | −1.76 | −0.98 | −0.68 | 0.87 |
| | | 2- | | | | | |
| | | aminopentanoic | | | | | |
| | | acid) | | | | | |
| 59 | Obser | O- | −5.20 | 2.54 | −0.60 | 0.32 | −0.48 |
| | | benzylserine | | | | | |
| 60 | Obtyr | O-benzyl- | −7.71 | 7.33 | −1.81 | 2.39 | 0.11 |
| | | tyrosine | | | | | |
| 61 | Oetyr | O- | −5.62 | 3.33 | −0.75 | 0.71 | −1.17 |
| | | ethyltyrosine | | | | | |
| 62 | Omser | O- | −1.02 | −0.30 | 0.36 | −0.97 | 1.70 |
| | | methylserine | | | | | |
| 63 | Omthr | O-methythreonine | −1.75 | −1.63 | −1.55 | −1.60 | −0.20 |
| 64 | Omtyr | O-methyl- | −4.28 | 3.05 | −0.03 | 0.72 | −1.11 |
| | | tyrosine | | | | | |
| 65 | Orn | ornithine | 3.09 | 0.17 | −1.85 | 1.46 | 0.42 |
| 66 | Pen | penicillamine | 0.15 | −0.76 | 0.42 | 0.67 | −2.79 |
| 67 | Pga | pyroglutamic | −3.56 | 2.88 | 2.82 | 1.09 | 3.10 |
| | | acid | | | | | |
| 68 | Pip | pipecolic acid | −2.66 | −2.29 | −1.57 | 0.20 | −0.39 |
| 69 | Sar | sarcosine | 0.30 | −3.55 | −0.09 | 0.29 | −0.35 |
| 70 | Tfa | 3,3,3- | −1.47 | 1.11 | 3.66 | −4.70 | 2.13 |
| | | trifluoro- | | | | | |
| | | alanine | | | | | |
| 71 | Thphe | 6- | 1.29 | 5.13 | 0.89 | −0.93 | −2.06 |
| | | hydroxydopa | | | | | |
| 72 | Vig | L-vinylglycine | −0.81 | 1.17 | 3.54 | 1.20 | 3.43 |
| 73 | AasPa | (−)-(2R)-2- | 5.35 | 6.24 | 2.92 | −1.44 | −2.26 |
| | | amino-3-(2- | | | | | |
| | | aminoethyl- | | | | | |
| | | sulfonyl)propanoic | | | | | |
| | | acid | | | | | |
| | | dihydro- | | | | | |
| | | chloride | | | | | |
| 74 | Ahdna | (2S)-2- | −1.40 | 3.33 | −2.51 | −2.81 | 1.96 |
| | | amino-9- | | | | | |
| | | hydroxy-4,7- | | | | | |
| | | dioxano- | | | | | |
| | | nanoic acid | | | | | |
| 75 | Ahoha | (2S)-2- | 0.05 | 1.17 | −0.74 | −1.96 | 1.64 |
| | | amino-6- | | | | | |
| | | hydroxy-4- | | | | | |
| | | oxahexanoic | | | | | |
| | | acid | | | | | |
| 76 | Ahsopa | (−)-(2R)-2- | 3.01 | 5.82 | 3.85 | −3.86 | −1.72 |
| | | amino-3-(2- | | | | | |
| | | hydroxyethylsulfonyl)- | | | | | |
| | | propanoic | | | | | |
| | | acid | | | | | |

Using the expanded descriptor scales as listed in table 1A, the inventors assembled the 5 individual average interval values Z_{Σ1}; Z_{Σ2}; Z_{Σ3}; Z_{Σ4} and Z_{Σ5} of 4 known cell-penetrating peptides (CPPs): transportan, penetratin, pVEC and MAP; averaged over the total number of amino acids in the sequence.

Z_{Σ1}, Z_{Σ2}, Z_{Σ3}, Z_{Σ4} and Z_{Σ5} average values of the respective descriptor values for the residues in said amino acid sequence, calculated with the formula

Z_{Σx}=(Zₓᵣₑₛ₁+Zₓᵣₑₛ₂ . . . +Zₓᵣₑₛₙ)/n

obtained from the training set are listed in Table 1B.

TABLE 1B
Descriptor values for the QSAR training set

| Name | z₁ | z₂ | z₃ | z₄ | z₅ |
|---|---|---|---|---|---|
| Transportan | −0.728 | −0.992 | −0.575 | −0.308 | 0.64 |
| pVEC | 0.191 | −0.118 | −0.499 | −0.950 | 0.881 |
| penetratin | 0.157 | 1.073 | −0.586 | 0.167 | 0.296 |
| MAP | −0.948 | −1.03 | −1.071111 | 0.087 | 1.08 |

Consequently, the inventors were able to determine that a cell-penetrating amino acid fragment based on its Z_{Σ} bulk property value is characterised by having a Z_{Σ} bulk property value essentially consisting of individual average interval values, wherein Z_{Σ1}<0.2; Z_{Σ2}<1.1; Z_{Σ3}<−0.49; Z_{Σ4}<0.33; and Z_{Σ5}<1.1 and Z_{Σ5}>0.12.

The present invention thus discloses a first method for predicting, detecting and/or verifying a potential cell-penetrating peptide, comprising obtaining the amino acid sequence of a protein or peptide, selecting an amino acid sequence of at least one candidate fragment and assessing the bulk property value Z_{Σ} of said sequence, Z_{Σ1} comprising at least 5 individual average interval values Zen; Z_{Σ2}; Z_{Σ3}; Z_{Σ4} and Z_{Σ5}, wherein Z_{Σ1}, Z_{Σ2}, Z_{Σ3}, Z_{Σ4} and Z_{Σ5} are average values of the respective descriptor values for the residues in said amino acid sequence, calculated with the formula

Z_{Σx}=(Zₓᵣₑₛ₁+Zₓᵣₑₛ₂ . . . +Zₓᵣₑₛₙ)/fn

Z_{xresy} being the respective descriptor value for amino acid residue y comprised in the selected candidate fragment, and wherein the descriptor value of each residue corresponds to a Z₁, Z₂, Z₃, Z₄, and Z₅ descriptor value in a descriptor value scale as listed in table 1A, and identifying a cell-penetrating fragment from said at least one candidate fragment(s) based on its Z_{Σ} bulk property value. A cell-penetrating fragment is herein characterised by having a Z_{Σ} bulk property value essentially consisting of individual average interval values, wherein Z_{Σ1}<0.2; Z_{Σ2}<1.1; Z_{Σ3}<−0.49; Z_{Σ4}<0.33; and Z_{Σ5}<1.1 and Z_{Σ5}>0.12. Optionally, said cell-penetrating capacity of said identified peptide or protein and/or said fragment is further verified by in vitro and/or in vivo methods.

What is more, by determining for the first time the necessary qualities for a given peptide to display cell-penetrating activity, the present invention consequently also makes it possible to design an abundance of CPPs de novo, which do not necessarily have to be modelled on a naturally occurring counterpart, but can be designed to take into account any other desired parameter, such as obeying a certain size, insolubility in a given pH environment, degradability in the body, or biological effect on a given target cell/tissue or organ.

In another, presently preferred embodiment of the present invention, the above selection criteria are furthermore supplemented by a three grade system for successive narrowing of the descriptor interval, and two additional descriptors are introduced: Bulkₕₐ being the number of non-hydrogen atoms (C, A, S and O) in the side chains of the amino acids, and hdb standing for the number of accepting hydrogen bonds for the side chains of the amino acids.

TABLE 10
New descriptors:

| Amino acid | Bulkₕₐ | hdb |
|---|---|---|
| A | +1 | 0 |
| C | +2 | 0 |
| D | +4 | −4 |
| E | +5 | −4 |
| F | +7 | 0 |
| G | 0 | 0 |
| H | +6 | −2 |
| I | +4 | 0 |
| K | +5 | +3 |
| L | +4 | 0 |
| M | +4 | 0 |
| N | +4 | 0 |
| P | +3 | 0 |
| Q | +5 | 0 |
| R | +7 | +5 |
| S | +2 | −1 |
| T | +3 | −1 |
| V | +3 | −0 |
| W | +10 | +1 |
| Y | +8 | 0 |

The supplemented selection criteria uses Z_{Σ1}, Z_{Σ2}, Z_{Σ3}, Z_{ΣBulkha} and net hydrogen bond donation (hdb) and average hdb Z_{Σhdb}.

Bulkha is calculated as number of atoms in a side chain of the amino acids, not counting hydrogens. E.g. for CH₂CH₂OH (serine) Bulkₕₐ=2*C+1*O=3. Z_{ΣBukha} is the average value of the number of atoms in side chains of the amino acids, not counting hydrogens, for the residues in said amino acid sequence, calculated with the formula

Z_{Σ Bulkha}=(Z_{Bulkha res1}+Z_{Bulkha res2} . . . +Z_{Bulkha resn})/n

Z_{Bulkha resy} being the respective Bulkₕₐ value for amino acid residue y comprised in the selected candidate fragment.

hdb is calculated as the donated hydrogen bonds-accepted hydrogen bonds of side chains. E.g. N—H donates and C═O accepts. There are 2 uses of hydrogen bonds in the same set, the total (hdb) and the average (Z_{Σhdb}). Z_{Σhdb} is the average value of the number of donated hydrogen bonds-accepted hydrogen bonds of side chains, for the residues in said amino acid sequence, calculated with the formula

Z_{Σhdb}=(Z_{hdb res1}+Z_{hdb res2} . . . +Z_{hdb resn})/n

Z hdb resy being the respective hdb value for amino acid residue y comprised in the selected candidate fragment.

The three grades represent a successive narrowing of the descriptor interval. The performance of the grades can be seen from table 11. In short, the higher the grade, the lower the chance that a predicted CPP is a “false” positive. Thus, an amino acid sequence falling into grade 3 (>2), is to a substantially higher degree expected to exhibit a cell-penetrating ability when subsequently tested either in vivo or in vitro.

TABLE 11
Correctly predicted, in %, for the 3 grades

| | Grade is | | |
|---|---|---|---|
| | >0 | >1 | >2 |
| Positives | 100 | 72 | 41 |
| Negative | 50 | 61 | 82 |
| Unrelated | 80 | 90 | 100 |

Positives correspond to the CPP training-set, negatives correspond to the non functional CPP analogues training-set and unrelated correspond to hormone training-set. See table 12 “training sets”0.2

TABLE 12

| | Non-functional-CPP-analogues | |
|---|---|---|
| CPP training-set | training-set | Hormone training-set |
| GWTLNSAGYLLGKINLKALAALAKKIL | GWTLNSAGYLLGKFLPLILRKIVTAL | QNLGNQWAVGHLM |
| RQIKIWFQNRRMKWKK | LLGKINLKALAALAKIL | RPPGFSPFR |
| KLALKALKALKAALKLA | LNSAGYLLGKALAALAKKIL | LYGNKPRRPYIL |
| LLIILRRRIRKQAHAHSK | LNSAGYLLGKLKALAALAK | GWTNLSAGYLLGPPPGFSPFR |
| AGYLLGKINLKALAALAKKIL | GWTLNSAGYLLGKINLKAPAALAKKIL | GWTLNSAGYLLGPHAI |
| FLGKKFKKYFLQLLK | LLKTTALLKTTALLKTTA | HDEFERHAEGTFTSDVSSYLEGQAA |
| | | KEFIAWLVKGR |
| GRKKRRQRRRPQ | LLKTTELLKTTELLKTTE | WSYGLRPG |
| RRRRRRRRR | GRKKRRQPPQC | TIHCKWREKPLMLM |
| GWTLNSAGYLLGKINLKALAALAKKLL | FITKALGISYGRKKRRQC | FVPIFTHSELQKIREKERNKGQ |
| GWTLNPAGYLLGKINLKALAALAKKIL | QNLGNQWAVGHLM | AGCKNFFWKTFTSC |
| GWTLNPPGYLLGKINLKALAALAKKIL | RPPGFSPFR | CYFQNCPRG |
| LNSAGYLLGKINLKALAALAKKIL | LYGNKPRRPYIL | |
| GWTLNSAGYLLGKLKALAALAKKIL | GWTNLSAGYLLGPPPGFSPFR | |
| RRWRRWWRRWWRRWRR | GIWFAYSRGHFRTKKGT | |
| GWTLNSKINLKALAALAKKIL | LRKKKKKH | |
| LNSAGYLLGKLKALAALAKIL | VATIKSVSFYTRK | |
| AGYLLGKLKALAALAKKIL | KKKQYTSIHHGVVEVD | |
| KLALKLALKALKAALK | RQIKIFFQNRRMKFKK | |
| KLALKLALKAWKAALKLA | KKLSECLKRIGDELDS | |
| KITLKLAIKAWKLALKAA | PVVHLTLRQAGDDFSR | |
| KALAKALAKLWKALAKAA | EILLPNNYNAYESYKYPGMFIALSK | |
| KALKKLLAKWAAAKALL | IAARIKLRSRQHIKLRHL | |
| KLAAALLKKWKKLAAALL | | |
| LKTLATALTKLAKTLTTL | | |
| KALAALLKKWAKLLAALK | | |
| KLALKLALKALQAALQLA | | |
| KLALQLALQALQAALQLA | | |
| QLALQLALQALQAALQLA | | |
| LLKKRKVVRLIKFLLK | | |
| RLIKTLKTLLQKRKTL | | |
| NAKTRRHERRRKLAIER | | |
| LLIILRRPIRKQAHAHSK | | |
| LLIILRARIRKQAHAHSK | | |
| LLIILRRRIRKQAHAHSA | | |
| TRRNKRNRIQEQLNRK | | |
| GGRQIKIWFQNRRMKWKK | | |
| MGLGLHLLVLAAALQGAKKKRKV | | |
| RKKRRQRRR | | |
| GRKKRRQRRRPPC | | |
| GRKKRRQRRRC | | |
| GRKKRRQRRPPQC | | |
| RQPKIWFPNRRMPWKK | | |
| RQIKIWFPNRRMKWKK | | |
| TRQARRNRRRWRERQR | | |
| KMTRAQRRAAARRNRWTAR | | |
| RVIRVWFQNKRCKDKK | | |
| RKSSKPIMEKRRRAR | | |
| YGRKKRRQRRRPPLRKKKKKH | | |
| RQIKIWFQNRRMKWKKLRKKKKKH | | |
| VQAILRRNWNQYKIQ | | |
| MAQDIISTIGDLVKWIIDTVNKFTKK | | |
| KRPAATKKAGQAKKKKL | | |
| RRRRNRTRRNRRRVR | | |
| TRRQRTRRARRNR | | |
| MDAQTRRRERRAEKQAQWKAAN | | |
| TAKTRYKARRAELIAERR | | |
| RQGAARVTSWLGRQLRIAGKRLEGR | | |
| SK | | |
| RQGAARVTSWLGRQLRIAGKRLEGR | | |
| GAARVTSWLGRQLRIAGKRLEGRSK | | |
| RVTSWLGRQLRIAGKRLEGRSK | | |
| SWLGRQLRIAGKRLEGRSK | | |
| GRQLRIAGKRLEGRSK | | |
| KCRKKKRRQRRRKKLSECLKRIGDE | | |
| LDS | | |
| KCRKKKRRQRRRKKPVVHLTLRQAG | | |
| DDFSR | | |
| AAVALLPAVLLALLAPVQRKRQKLMP | | |
| RRRRRRWGRWGRWGRWGRWGR | | |
| WGRPKKKRKV | | |
| ALWMTLLKKVLKAAAKAALNAVLVG | | |
| ANA | | |
| ALWKTLLKKVLKA | | |
| PKKKRKVALWKTLLKKVLKA | | |
| RQARRNRRRALWKTLLKKVLKA | | |
| RQARRNRRRC | | |
| RRLSYSRRRF | | |
| RGGRLSYSRRRFSTSTGR | | |
| YGRKKRRQRRRSVYDFFVWL | | |
| YGRKKRRQRRRGTSSSSDELSWIIE | | |
| LLEK | | |
| IVIAKLKA | | |

As described previously for the first set of selection criteria, the values for the peptide are averaged (divided by number of amino acid residues in the peptide). Z_{Σ1}, Z_{Σ2}, Z_{Σ3}, Z_{ΣBulkha}, Z_{Σrhdb}

The intervals for the different grades are:

3: Preferred: Z_{ΣBulkha}>3.1 and Z_{ΣBulkha}<8.13 and Z_{Σ1}>−1.25 and Z_{Σ1}<3.52 and Z_{Σ2}>−3.9 and Z_{Σ2}<3.1 and Z_{Σ3}<−0.5 and Z_{Σ1}3>−3.51 and Z_{Σrhdb}>−0.115 and Z_{Σhdb}<5.1 and hdb>0 and hdb<84
2: More preferred: Z_{ΣBulkha}>3.2 and Z_{ΣBulkha}<5.9 and Z_{Σ1}>−1.25 and Z_{Σ1}<1.92 and Z_{Σ2}>−1.22 and Z_{Σ2}<1.29 and Z_{Σ3}<−0.5 and Z_{Σ3}>−1.94 and Z_{Σhdb}>0.28 and Z_{Σhdb}<2 and hdb>5 and hdb<30
1: Most preferred: Z_{ΣBulkha}>3.2 and Z_{ΣBulkha}<4.8 and Z_{Σ1}>−1.1 and Z_{Σ1}<1.92 and Z_{Σ2}>−1.1 and Z_{Σ2}<0 and Z_{Σ3}<−0.55 and Z_{Σ3}>−1.94 and Z_{Σhdb}>−0.28 and Z_{Σhdb}<1.57 and hdb>7 and hdb<25

As has been demonstrated for several of the serendipitously found CPPs, as for e.g. Transportan, penetratin and tat, truncation of the original sequence can still give an active CPP. This indicates that the previously found CPPs might contain a shorter sequence acting as the transporter “motor”. Taking this into account, a second aspect of the invention is directed to a method for checking cellular penetration properties of a peptide, or a shorter fragment of a peptide, such as from a known CPP, comprising the steps of obtaining the amino acid sequence of the peptide, assessing the bulk property value Z_{Σ} of said sequence, Z_{Σ} comprising at least 5 individual average interval values Z_{Σ1}; Z_{Σ2}; Z_{Σ3}; Z_{Σ4} and Z_{Σ5}, wherein Z_{Σ1}, Z_{Σ2}, Z_{Σ3}, Z_{Σ4} and Z_{Σ5} are average values of the respective descriptor values for the residues in said amino acid sequence, calculated with the formula

Z_{Σx}=(Zₓᵣₑₛ₁+Zₓᵣₑₛ₂ . . . +Zₓᵣₑₛₙ)/n

Z_{xresy} being the respective descriptor value for amino acid residue y comprised in the selected candidate fragment, and wherein the descriptor value of each residue corresponds to a Z₁, Z₂, Z₃, Z₄, and Z₅ descriptor value in a descriptor value scale as listed in table 1A, and checking the cell-penetrating properties of said peptide based on its Z₄ bulk property value, wherein a cell-penetrating fragment is characterised by having a Z₄ bulk property value essentially consisting of individual average interval values, wherein Z_{Σ}Z_{Σ1}<0.2; Z_{Σ2}<1.1; Z_{Σ3}<−0.49; Z_{Σ4}<0.33; and Z_{Σ5}<1.1 and Z_{93 5}>0.12, synthesizing or Isolating a peptide comprising the amino acid sequence of said Identified cell-penetrating peptide, and optionally verifying the protein-mimicking functionality and/or the cell-penetrating capacity of the synthesized or Isolated peptide by in vitro and/or in vivo methods. And optionally again, one may supplement the above selection criteria by a three grade system for successive narrowing of the descriptor interval, introducing two additional descriptors as described: Bulkₕₐ being the number of non-hydrogen atoms (C, N, S and O) in the side chains of the amino acids, and hdb standing for the number of accepting hydrogen bonds for the side chains of the amino acids.

Also comprised in the present invention is a method for producing a cell-penetrating and functional protein-mimicking peptide, essentially comprising the steps of selecting a functional protein of interest, obtaining the amino acid sequence of said selected protein, selecting the amino acid sequence of at least one candidate fragment corresponding to an intracellular part of said protein, assessing the bulk property value Z_{Σ4} of said sequence, Z_{Σ4} comprising at least 5 individual average interval values Z_{Σ1}; Z_{Σ2}; Z_{Σ3}; Z_{Σ4} and Z_{Σ5}, wherein Z_{Σ1}, Z_{Σ2}, Z_{Σ3}, Z_{Σ4} and Z_{Σ5} are average values of the respective descriptor values for the residues in said amino acid sequence, calculated with the formula

Z_{Σx}=(Zₓᵣₑₛ₁+Zₓᵣₑₛ₂ . . . +Zₓᵣₑₛₙ)/n

Z_{xresy} being the respective descriptor value for amino acid residue y comprised in the selected candidate fragment, and wherein the descriptor value of each residue corresponds to a Z₁, Z₂, Z₃, Z₄, and Z₅ descriptor value in a descriptor value scale as listed in table 1A, and identifying a cell-penetrating fragment from said at least one candidate fragment(s) based on its Zy bulk property value, wherein a cell-penetrating fragment is characterised by having a Z bulk property value essentially consisting of individual average interval values, wherein Z_{Σ1}<0.2; Z_{Σ1}2<1.1; Z_{Σ3}<−0.49; Z_{Σ4}<0.33; and Z_{Σ5}<1.1 and Z_{Σ5}>0.12. Finally, synthesizing or isolating a peptide comprising the amino acid sequence of said Identified cell-penetrating peptide, and optionally, verifying the protein-mimicking functionality and/or the cell-penetrating capacity of the synthesized or Isolated peptide by in vitro and/or in vivo methods. And optionally, supplementing the above selection criteria by a three grade system for successive narrowing of the descriptor interval, introducing two additional descriptors as described: Bulkₕₐ being the number of non-hydrogen atoms (C, N, S and O) in the side chains of the amino acids, and hdb standing for the number of accepting hydrogen bonds for the side chains of the amino acids.

In yet another preferred embodiment, the present invention comprises designing and producing a CPP peptide or fragment de novo, wherein said fragment can either resemble a naturally occurring CPP, and/or be designed to mimic a naturally occurring cellular effector peptide, or be designed essentially randomly, mainly taking into account a predicted cell penetration capability of a random amino acid sequence of a given length, comprising the steps of designing the amino acid sequence of said sequence, assessing the bulk property value Z_{Σ} of said sequence, Z_{Σ} comprising at least 5 Individual average interval values Z_{Σ1}; Z_{Σ2}; Z_{Σ3}; Z_{Σ4} and Z_{Σ5}, wherein Z_{Σ1}: Z_{Σ2}, Z_{Σ3}, Z_{Σ4} and Z_{Σ5} are average values of the respective descriptor values for the residues in said amino acid sequence, calculated with the formula

Z_{Σx}=(Zₓᵣₑₛ₁+Zₓᵣₑₛ₂ . . . +Zₓᵣₑₛₙ)/n

Z_{xresy} being the respective descriptor value for amino acid residue y comprised in the selected candidate fragment, and wherein the descriptor value of each residue corresponds to a Z₁, Z₂, Z₃, Z₄, and Z₅ descriptor value in a descriptor value scale as listed in table 1A, and checking the cell-penetrating properties of said peptide based on its Z_{Σ} bulk property value, wherein a cell-penetrating fragment is characterised by having a Z_{Σ} bulk property value essentially consisting of Individual average interval values, wherein Z_{Σ1}<0.2; Z_{Σ2}<1.1; Z_{Σ3}<−0.49; Z_{Σ4}<0.33; and Z_{Σ5}<1.1 and Z_{Σ5}>0.12, synthesizing or isolating a peptide comprising the amino acid sequence of said identified cell-penetrating peptide, and optionally verifying the protein-mimicking functionality and/or the cell-penetrating capacity of the synthesized or isolated peptide by in vitro and/or in vivo methods. And optionally again, supplementing the above selection criteria by a three grade system for successive narrowing of the descriptor interval, introducing two additional descriptors as described: Bulkₕₐ being the number of non-hydrogen atoms (C, N, S and O) in the side chains of the amino acids, and hdb standing for the number of accepting hydrogen bonds for the side chains of the amino acids.

Another, equally preferred, embodiment of the present invention relates to a method for producing an artificial cell-penetrating peptide and/or an artificial cell-penetrating and functional protein-mimicking peptide, comprising the steps of designing at least one artificial peptide and/or peptide fragment, assessing the bulk property value Z_{Σ} of the amino acid sequence of said artificial peptide or peptide fragment, Z_{Σ} comprising at least 5 individual average interval values Z_{Σ1}; Z_{Σ2}; Z_{Σ3}; Z_{Σ4} and Z_{Σ5} wherein Z_{Σ1}, Z_{Σ2}, Z_{Σ2}, Z_{Σ3}, Z_{Σ4} and Z_{Σ5} are average values of the respective descriptor values for the residues in said amino acid sequence, calculated with the formula

Z_{Σx}=(Zₓᵣₑₛ₁+Zₓᵣₑₛ₂ . . . +Zₓᵣₑₛₙ)/fn

Z_{xresy} being the respective descriptor value for amino acid residue y comprised in the selected candidate fragment, and wherein the descriptor value of each residue corresponds to a Z₁, Z₂, Z₃, Z₄, and Z₅ descriptor value in a descriptor value scale as listed in table 1A, and checking the cell-penetrating properties of said artificial peptide and/or peptide fragment based on its Z_{Σ} bulk property value, wherein a cell-penetrating fragment is characterised by having a Z_{Σ} bulk property value essentially consisting of individual average interval values, wherein Z_{Σ1}<0.2; Z_{Σ2}<1.1; Z_{Σ3}<−0.49; Z_{Σ5}<0.33; and Z_{Σ5}<1.1 and Z_{Σ5}>0.12. Further synthesizing said peptide and/or peptide fragment comprising the amino acid sequence identified as cell penetrating, and optionally verifying the protein-mimicking functionality and/or the cell-penetrating capacity of the synthesized peptide and/or peptide fragment by in vitro and/or in vivo methods. And optionally again, supplementing the above selection criteria by a three grade system for successive narrowing of the descriptor interval, introducing two additional descriptors as described: Bulkₕₐ being the number of non-hydrogen atoms (C, N, S and O) in the side chains of the amino acids, and hdb standing for the number of accepting hydrogen bonds for the side chains of the amino acids.

In the present context, a cell-penetrating fragment is characterised by having a Z_{Σ} bulk property value essentially consisting of individual average Interval values, wherein most preferably Z_{Σ1}<0.2; Z_{Σ2<1.1}; Z_{Σ3}<−0.49; Z₄<0.33; and Z_{Σ5}<1.1 and Z_{Σ5}>0.12. In alternative embodiments of the invention, said individual average values can comprise

Z_{Σ1}<0.3, such as Z_{Σ1}<0.21, Z_{Σ1}<0.22, Z_{Σ1}<0.23, Z_{Σ1}<0.24, Z_{Σ1}<0.25, Z_{Σ1}<0.26,

### Z_{Σ1}<0.27, Z_{Σ1}<0.28, or Z_{Σ1}<0.29;

Z_{Σ2}2<1.2, such as Z_{Σ2}<1.11, Z_{Σ2}<1.12, Z_{Σ2}<1.13, Z_{Σ2<1.14}, Z_{Σ2}<1.15, Z_{Σ2}<1.16, Z_{Σ2}<1.17, Z_{Σ2}<1.18, or Z_{Σ2}<1.19;
Z_{Σ3}<−0.39, such as Z_{Σ3}<−0.4, Z_{Σ3}<−0.41, Z_{Σ3}<−0.42, Z_{Σ3}<−0.43, Z_{Σ3}<−0.45, Z_{Σ3}<0.46, Z_{Σ3}<−0.47, or Z_{Σ3}<−0.48;
Z_{Σ4}<0.43, such as Z_{Σ4}<0.34, Z_{Σ4}<0.35, Z_{Σ4}<0.36, Z_{Σ4}<0.37, Z_{Σ4}<0.38, Z_{Σ4}<0.39, Z_{Σ4}<0.4, Z_{Σ4}<0.41, or Z_{Σ4}<0.42;
Z_{Σ5}<1.05 and Z_{Σ5}>0.22, such as Z_{Σ5}<1.04 and Z_{Σ5}>0.21, Z_{Σ5}<1.03 and Z_{Σ5}>0.20,

### Z_{Σ5}<1.02 and Z_{Σ5}>0.19, Z_{Σ5}<1.01 and Z_{Σ5}>0.11, Z_{Σ5}<1.00 and Z_{Σ5}>0.17, Z_{Σ5}<0.99 and

### Z_{Σ5}>0.16, Z_{Σ5}<0.98 and Z_{Σ5}>0.15, Z_{Σ}<0.97 and Z_{Σ5}>0.14, or Z_{Σ5}<0.96 and Z_{Σ5}>0.13.

In a presently preferred embodiment, said cell-penetrating fragment is further, or alternatively characterised by descriptor values as described above, essentially consisting of individual average interval values, wherein most preferably Z_{ΣBulkha}>3.1 and Z_{ΣBulkha}<8.13, and Z_{Σ1}>−1.25 and Z_{Σ1}<3.52, and Z_{Σ2}>−3.9 and Z_{Σ2}<3.1, and Z_{Σ3}<−0.5 and Z_{Σ3}>−3.51, and Z_{Σhdb}>−0.115 and Z_{Σhdb}<5.1, and hdb>0 and hdb<84;

or Z_{ΣBulkha}>3.2 and Z_{ΣBulkha}<5.9, and Z_{Σ1}>−1.25 and Z_{Σ1}<1.92, and Z_{Σ2}>−1.22 and Z_{Σ2}<1.29, and Z_{Σ3}<−0.5 and Z_{Σ3}>−1.94, and Z_{Σhdb}>0.28 and Z_{Σhdb}<2, and hdb>5 and hdb<30,
and most preferred; Z_{ΣBulkha}>3.2 and Z_{ΣBulkha}<4.8, and Z_{Σ1}>−1.1 and Z_{Σ1}<1.92, and Z_{Σ2}>−1.1 and Z_{Σ2}<0, and Z_{Σ3}<−0.55 and Z_{Σ3}>−1.94, and Z_{Σhdb}>−0.28 and Z_{Σhdb}<1.57, and hdb>7 and hdb<25.

Additionally, any conservative variant of the sequence of a CPP found, designed and/or verified by a method according to the present invention, and any cell membrane penetrating analogues of a CPP found, designed and/or verified by a method according to the present invention, is by virtue of its functional relationship to said CPP considered to be inside the scope of the present invention.

A conservative variant of a sequence is in the present context defined as an amino acid sequence which is conserved at least 70%, such as 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, when comparing variants of the same amino acid sequence between different species. The degree of conservation of a variant can, as is well known in the field, be calculated according to its derivation of PAM (see Dayhoff, Schwartz, and Orcutt (1978) Atlas Protein Seq. Struc. 5:345-352), or based on comparisons of Blocks of sequences derived from the Blocks database as described by Henikoff and Henikoff (1992) Proc Natl Acad Sci USA 89(22):10915-9.

Conservative substitutions may be made, for example according to table 17 below. Amino acids in the same block in the second column and preferably in the same line in the third column may be substituted for each other:

TABLE 17

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Such replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-g-amino butyric acid*, L-α-amino isobutyric acid*, L-e-amino caproic acid#, 7-amino heptanoic acid*, L-methionine sulfone#*, L-norleucine*, L-norvaline*, p-nitro-L-phenylalanine*, L-hydroxyproline#, L-thloproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tlc (1,2,3,4-tetrahydroisoquinoline-3-carboxyl acid)*, L-diaminopropionic acid # and L-Phe (4-benzyl)*. The notation* is herein utilised to indicate the hydrophobic nature of the derivative whereas # is utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or b-alanine residues. A further form of variation, involves the presence of one or more amino acid residues in peptoid form, which will be well understood by those skilled in the art. For the avoidance of doubt, “the peptoid form” is used to refer to variant amino acid residues wherein the a-carbon substituent group is on the residue's nitrogen atom rather than the α-carbon. Processes for preparing peptides in the peptoid form are known in the art, see for example, Simon R J et al., PNAS (1992) 89 (20), 9367-9371 and Horwell D C, Trends Biotechnol. (1995) 13(4), 132-134.

Peptides of the invention may be in a substantially isolated form. It will be understood that the peptide may be mixed with carriers or diluents, which will not interfere with the intended purpose of the peptide and still be regarded as substantially Isolated. A peptide of the invention may also be in a substantially purified form, in which case it will generally comprise the peptide or a fragment thereof in a preparation in which more than 90%, e.g. 95%, 98% or 99% of the protein in the preparation is a peptide of the invention.

Furthermore, any amino acid sequence being at least 70% identical, such as being at least 72%, 75%, 77%, 80%, 82%, 85%, 87%, 90%, 91%, 92%, 93%, 94%, 95%, 96%, 97%, 98%, or 99% identical with the amino acid sequence of a CPP, characterised by having a Z_{Σ} bulk property value essentially consisting of individual average interval values, wherein Z_{Σ1}<0.2; Z_{Σ2}<1.1; Z_{Σ3}<−0.49; Z_{Σ4}<0.33; and Z_{Σ5}<1.1 and Z_{Σ5}>0.12, or fitting into grade 1, 2, or 3, assessed as discussed above, found, designed and/or verified by a method according to the present invention, is also considered to be inside the scope of the present invention.

By a polypeptide having an amino acid sequence at least, for example 95% identical to a reference amino acid sequence, is intended that the amino acid sequence of the polypeptide is identical to the reference sequence except that the amino acid sequence may include up to 5 point mutations per each 100 amino acids of the reference amino acid sequence. In other words, to obtain a polypeptide having an amino acid sequence at least 95% identical to a reference amino acid sequence: up to 5% of the amino acids in the reference sequence may be deleted or substituted with another amino acid, or a number of amino acids up to 5% of the total amino acids in the reference sequence may be inserted into the reference sequence. These mutations of the reference sequence may occur at the amino or carboxy terminal positions of the reference amino acid sequence or anywhere between those terminal positions, interspersed either individually among amino acids in the reference sequence or in one or more contiguous groups within the reference sequence.

In the present invention, a local algorithm program is best suited to determine identity. Local algorithm programs, such as (Smith-Waterman) compare a subsequence in one sequence with a subsequence in a second sequence, and find the combination of subsequences and the alignment of those subsequences, which yields the highest overall similarity score. Internal gaps, if allowed, are penalized. Local algorithms work well for comparing two multidomain proteins, which have a single domain, or just a binding site in common.

Methods to determine Identity and similarity are codified in publicly available programs. Preferred computer program methods to determine identity and similarity between two sequences include, but are not limited to, the GCG program package (Devereux, J., et al., Nucleic Acid Research 12 (1):387 (1984)), BLASTP, BLASTN, and FASTA (Altschul, S. F., et al., J. Molec. Bio 1.215:403-410 (1990)). The BLASTX program is publicly available from NCBI and other sources (BLAST Manual, Altschul, S. F., et al., NCBI NLM NIH Bethesda, Md. 20894; Altschul, S. F., et al., J. Molec. Bio 1.215:403-410 (1990)). Each sequence analysis program has a default scoring matrix and default gap penalties. In general, a molecular biologist would be expected to use the default settings established by the software program used.

In the present context, an amino acid is any organic compound containing an amino (—NH₂) and a carboxyl (—COOH) group. Amino acids can be in either L- or D-form. There are at present 22 known coded α-amino acids from which proteins are synthesized during ribosomai transiation of mRNA. Additionally, a vast number of non-coded amino adds are constantly emerging, of which 56 examples are given in table 1A. Both coded and non-coded amino acids can of course be part of the amino acid sequences, peptide fragments, peptides, proteins and/or polypeptides included in the present invention.

Amino acid sequence is in the present context the precisely defined linear order of amino acids (including both coded and/or non-coded amino acids) in a peptide fragment, peptide, protein or polypeptide.

The term non-peptide analogue is in the present context employed to describe any amino acid sequence comprising at least one non-coded amino acid and/or having a backbone modification resulting in an amino acid sequence without a peptide linkage, i.e. a CO—NH bond formed between the carboxyl group of one amino acid and the amino group of another amino acid.

Furthermore, the present amino acid sequences may either be amidated or occur as free acids.

Reporter groups of different characters can be coupled to a putative CPP in order to estimate its cellular translocation and efficiency, such as biotin and different fluorophores, e.g. fluorescein, aminobenzoic acid, and rhodamines.

The present invention discloses a method for verifying the cell-penetrating capacity of a novel CPP and/or a known but improved CPP, which is efficient, fast and reliable, for screening the cellular uptake of a broad variety of CPPs in vitro and in vivo. The present invention thus in one embodiment relates to a method, wherein the cell-penetrating capacity of a peptide and/or peptide fragment is verified by monitoring the cellular uptake rate of said peptide into said cell after exposure to said peptide and/or peptide fragment. In a particularly applicable embodiment, the invention relates to a method, wherein the CPP itself is coupled to a traceable marker, such as a fluorescence detection marker, which can be detected by staining the target cells immunohistologically after cellular uptake has taken place.

Illustrative examples of said methods are described in example 10 and In example 16.

One aspect of the present invention thus comprises a method for screening the cellular uptake of a broad variety of CPPs in vitro, wherein cells are grown on glass cover slips to suitable, such as 50%, density, whereupon the media is changed to serum free and a blotinylated peptide solution is added. The cells are then incubated, washed, fixed and the peptides are visualised by staining with, e.g. avidin-FITC, or streptavidin-TRITC, and the cell nuclei are counterstained, e.g. with Hoechst. Images are preferably obtained with a fluorescence microscope and evaluated.

The term “cell-penetrating capacity” of a peptide will henceforth be used synonymously to its capability to translocate across the plasma membrane into either cytoplasmic and/or nuclear compartments of eukaryotic and/or prokaryotic cells, such as into cytoplasm, nucleus, lysosome, endoplasmatic reticulum, golgi apparatus, mitocondria and/or chloroplast, seemingly energy-independently. Additionally, the term “cell-penetrating capacity” of a peptide can in some aspects of the invention also be used synonymously to indicate transcellular or transmembrane transport, and thus also stand for e.g. the capability to translocate across an epithelial membrane, such as across the epithelium in the intestinal/buccal system, the mucosa in the mouth, lung, rectum or nose, or the blood-brain barrier of a mammal.

A detected or de novo designed and verified peptide, displaying cellular penetration capacity according to the present invention is in the present context defined as a “cell penetrating peptide (CPP)” and can e.g. be used for intracellular delivery of macromolecules, such as polypeptides and/or oligonucleotides with molecular weights several times greater than its own.

In general, cellular delivery using a cell-penetrating peptide and/or a non-peptide analogue thereof is non-invasive, energy-independent, efficient for a broad range of cell types and/or a broad variety of cargo, applicable to cells en masse, non-saturable, and/or receptor independent.

CPPs detected or de novo designed, and/or verified by a method disclosed in the present application will be useful for the transport of hydrophilic macromolecules into the cytoplasmic and nuclear compartments of a living cell and/or microorganism, without permanently disrupting the plasma membrane, as well as for delivering hydrophilic macromolecules across the blood-brain barrier, permitting e.g. the intracellular transport of conjugated oligopeptides and oligonucleotides and drugs.

Thus, a cell-penetrating peptide and/or a non-peptide analogue thereof might in the present context be used as a delivery vector for any pharmacologically interesting substance, such as a peptide, polypeptide, protein, small molecular substance, drug, mononucleotide, oligonucleotide, polynucleotide, antisense molecule, double stranded as well as single stranded DNA, RNA and/or any artificial or partly artificial nucleic acid, e.g. PNA, as well as a research tool for delivering e.g. tags and markers and/or for changing membrane potentials and/or properties.

A CPP found or designed and/or produced according to the present invention can therefore be of use as a vector for the delivery of a hydrophilic biomolecule and/or drug into cytoplasmic and nuclear compartments of a cell and/or a tissue, both in vivo and in vitro.

When covalently linked with a cargo, including any peptide, polypeptide, protein, small molecular substance, drug, polypeptide and oligonucleotide, with many times its own molecular mass, a CPP might still be able to translocate.

What is more, a CPP can in itself display intra and/or extracellular effector activity, thus unction as a cell-penetrating functional protein-mimicking peptide, or even display a new, on-predictable function when designed de novo.

A variety of different molecules have been presented in the technical field that are generated from naturally occurring cellular effectors, and that have been shown to essential retain the biological activity of its original counterpart after translocation into target cells (see e.g. Kuliopulos et al. WO0181408; or Covic et al., 2002, PNAS Vol. 99). Nonetheless, none of these have simultaneously been both CPP and biological effector. Typically, a truncated peptide is coupled to a known CPP or to a hydrophobic moiety, thus creating the necessity of various intermediate synthesis steps and a costly and complicated production.

A cell-penetrating, functional protein-mimicking peptide in the present context is a peptide that will, due to its Internal CPP capacity, be Internalised into a host cell, and once inside the host cell, will display a mimicking activity of either the original protein or peptide that it has been generated from, or a protein or peptide of choice to that it has been designed to mimic. A cell-penetrating, functional protein-mimicking peptide is thus defined as a CPP that in itself has effector activity and that will activate or inactivate an internal and/or external signalling pathway and/or cascade, resembling the activated functional protein that it is derived from. It is therefore characterised as having both cellular penetrating capability and effector and/or functional protein-mimicking activity.

A cellular effector can herein be either an intracellular and/or extracellular effector and is in the present context defined as a structure that produces a cellular effect, such as a contraction, secretion, electrical impulse, or activation or Inactivation of an intracellular and/or extracellular signalling cascade, or that induces the up regulation of a cellular level of an mRNA and/or a protein, in response to a stimulation by said effector. A typical effector is in the present context selected from the group consisting of a metabolite, an antagonist, an agonist, a receptor ligand, a receptor coupled protein, an activated receptor, an enzyme inhibitor, activator/inactivator and/or stimulator, a kinase, a phosphatase, an enhancer, or a silencer, a transcription factor, a transporter and/or a transmitter, a hormone, a channel, an ion, a prion, and a viral protein.

A typical CPP detected and verified by a method according to the present invention can be derived from or designed to resemble a broad variety of proteins and/or peptides. In one embodiment, said protein and/or peptide is a transmembrane protein, and In yet another embodiment, it can as well be a non-membrane-associated protein.

Consequently, one aspect of the present invention concerns a cell-penetrating functional protein-mimicking peptide, detected and verified by a method according to the present invention that is derived from a transcription factor or designed to closely resemble a transcription factor or at least a functional fragment of a transcription factor.

A preferred embodiment of the present invention thus relates to a CPP comprised in any of SEQ.ID.NO. 18399-31839, which are detected and verified by a method according to the present invention and as described above, and wherein each CPP is derived from a naturally occurring transcription factor.

Presently most preferred, though, the CPP detected and verified by a method according to the present invention, is derived from or designed to resemble a transmembraneous protein, such as a membrane-associated receptor or a receptor agonist and/or antagonist. A cell-penetrating functional protein-mimicking peptide is in this embodiment most preferably derived from a membrane-associated receptor or designed to closely resemble a membrane-associated receptor or at least a fragment of a membrane-associated receptor. More preferably still, the CPP is derived from an intracellular part or loop of said membrane-associated receptor.

As found for many G-protein coupled receptors, some synthetic peptides, derived from their intracellular loops, influence receptor-G-protein interactions in membrane preparation. Thus in a much preferred embodiment of the present invention, said cell-penetrating functional protein-mimicking peptide is derived from or designed to resemble a mammalian receptor, such as a receptor belonging to a protein family which can be classified based on its member's structure and their function and comprises channel receptors, tyrosine kinase receptors, guanylate cyclase receptors, serine/threonine kinase receptors, cytokine receptors, and receptors coupled to guanosine triphosphate (GTP)-binding proteins (G protein-coupled receptors: GPCRS).

GPCRs are in the present context defined as having seven transmembrane domains, three extracellular loops (e₁, e₂, e₃) and four intracellular loops (i₁, i₂, i₃, i₄). A cell-penetrating functional protein-mimicking peptide is thus preferably derived from or resembles a fragment of any of the intracellular or extracellular loops of said receptors.

In an even more preferred embodiment, said cell-penetrating functional protein-mimicking peptide is derived from or designed to resemble the group consisting of the GLP-1 receptor, AT1A receptor, CGRP receptor, and Dopamine-2 receptor.

Nonetheless, a cell-penetrating functional protein-mimicking peptide can equally well be derived from or be designed to resemble any other cellular effector, such as an enzyme, channel, hormone, transcription factor, receptor agonist or antagonist, transporter, or ligand, and can e.g. be derived from or resemble platelet-activating factor (PAF), CGRP, thyroid-stimulating hormone (TSH), luteinizing hormone (LH), or follicle-stimulating hormone (FSH).

A typical example for the above is given in example 13, wherein 24 peptides are synthesised resembling peptide fragments derived from different secretases believed to be involved in Aβ-production. The peptide sequences particularly comprised in the present invention are listed as SEQ.ID.NO. 31840-31864, and are derived from different secretases with the intention to produce a peptide containing an ability to bind to a consensus sequence in the secretase/APP, thus competing with the naturally occurring secretase binding, which at the same time is cell penetrating.

As described above, a CPP can stem from or be designed to mimic a receptor activating ligand, or an internal loop, or a transmembraneous loop of a receptor and thus have internal activating/repressing properties, but can also be solely transporting cargo across a membrane. Thus, in the present context, CPPs are divided into two classes:
a. functional protein-mimicking CPPs
b. cargo-transporting CPPs

None withstanding, a CPP belonging to group a) will of course in most cases also be capable and useful for cargo-transport.

The above described novel transport peptides, or any other receptor derived or resembling CPP, are universal transport peptides, functional protein-mimicking CPPs, as well as cargo-transporting CPPs, and can be used for cellular delivery of a variety of cellular effectors, e.g. general modifiers of intracellular and/or extracellular metabolic and signalling mechanisms, such as peptides, proteins, oligonucleotides and polynucleotides and/or for the delivery of antibiotics and/or antiviral agents into cells and microorganisms.

The following will exemplify a variety of different CPPs detected, or designed and verified with a method according to the present application. Given the magnitude of potential CPPs that can for the first time be detected, or designed with the different methods disclosed in the present application, naturally, the selection of specific and novel CPPs and improved usage of known CPPs given herein is purely meant to be illustrative and by no means exhaustive.

Examples for CPPs, detected and verified by a method according to the present application, are given in the experimental section, and are also solely intended to be illustrative and by no means exhaustive.

References mentioned in the present application are considered to be incorporated.

### Receptor Derived CPPs:

One preferred embodiment of the present invention comprises a novel synthesized peptide: GOP, derived from the glucagon like peptide 1 receptor, GLP-1 receptor (as described in detail in example 5). The novel CPP acts as a potent mimicker of action of the GLP-1 receptor, i.e. it increases Insulin release, when incubated with rat and human pancreatic islets. Further, the peptide and cell membrane penetrating analogues thereof are able to localize intracellularly, when incubated with cells and act as mimics of an agonist of GLP-1 receptor protein action. Thus, they are potential powerful candidates for treatment of non-insulin dependent diabetes mellitus, NIDDM, and generally for treating both diabetes type I and II.

Consequently, one specific aspect of the present invention is directed to a peptide selected from the group consisting of peptides comprising or essentially consisting of the amino acid sequence IVIAKLKA (GOP), conservative variants of the sequence and cell membrane penetrating analogues thereof.

The cell-penetrating analogues of peptide GOP, being in example 5 derived from rat GLP-1 receptor or de novo designed to resemble said receptor, such as GOP-6, which is a completely de novo designed sequence, wherein all amino acids are in D-form, or such as GOP-8, wherein all amino acids are N-methylated, can be seen in table 8, and are listed as SEQ.ID.NO. 31865-31886, may e.g. be corresponding peptides from other mammalian species or individual variants from the same species, and may thus have amino acid extensions, deletions or substitutions in relation to the amino acid sequence of peptide GOP, as long as they have cell-penetrating properties/capability. A representative example of this type of cell-penetrating peptides, held in the scope of the present invention, is IVIAKLKANLMCKTCRLAK-amide (M 569). Cell-penetrating properties of said analogues of GOP can easily be tested by a variety of standard methods, well known to the skilled artisan, or as illustrated in example 2 or 16.

The above described novel transport peptides, or any other receptor derived or resembling CPP, are universal transport peptides, functional protein-mimicking CPPs, as well as cargo-transporting CPPs, and can be used for cellular delivery of a variety of cellular effectors, e.g. general modifiers of intracellular and/or extracellular metabolic and signalling mechanisms, such as peptides, proteins, oligonucleotides and polynucleotides and/or for the delivery of antibiotics and/or antiviral agents into cells and microorganisms.

Another aspect of the invention is directed to the above disclosed CPP of the invention for use as a medicament, in particular a medicament for the treatment of Insulin deficiency in non-insulin dependent diabetes mellitus. Consequently, any other receptor-derived CPP can of course be used as a medicament for treating any disease or abnormal condition correlated to the receptor that said CPP is derived from. Typically, such diseases are selected from the group consisting of metabolic diseases or disorders, such as diabetes type I and type II, neurological diseases, such as Alzheimer's Disease, Huntington-Chorea, Parkinson's Disease, or epilepsy, taste and smell disorders, psycotic diseases, such as schitzophrenic diseases, depression, anxiety, a disease with oncogenic properties, ulcer, addiction and abuse disorders, Infectious diseases, inflammations, pain, Immunological diseases or disorders, such as asthma and allergy, immunological suppression, immunological hyper function, or autoimmune diseases.

In a presently preferred embodiment, wherein said CPP is derived from a G protein-coupled receptor, or designed to mimic a G protein-coupled receptor, said CPP is used for the manufacture of a pharmaceutical composition for the treatment of a disease with oncogenic properties, including toxic thyroid hyperplasia (mutated thyroid-stimulating hormone (TSH) receptor), retinis pigmentosa (mutated rhodopsin), precocious puberty (mutated luteinizing hormone (LH) receptor), hypocalcaemia (mutated Ca²⁺-receptor) and Jansen metaphyseal chondrodysplasia (mutated parathyroid hormone and parathyroid hormone-related peptides (PTH/PTHrP) receptors). Furthermore, said composition can also be used for the treatment of a pathology associated with Inactivation of GPCRs such as X-linked nephrogenic diabetes Insipidus (vasopressin V2 receptor), familial glucocorticoid deficiency (adrenal corticoid hormone (ACTH) receptor), bleeding disorder (thromboxane A₂ receptor), male pseudohermaphroditism (LH receptor), familial hypocalciuric hypercalcaemia, neonatal hyperparathyroidism (Ca²⁺ receptor) or Hirschprung disease (endothelin B receptor).

Still another aspect of the invention is directed to a method of treating insulin deficiency in a patient having non-insulin dependent diabetes mellitus, comprising the steps of administering to said patient an Insulin release increasing amount of a peptide according to the invention, or a pharmaceutical composition according to the invention. The Insulin release increasing amount will be recommended by the attending physician with guidance from the manufacturer and the response from the patient.

Yet another aspect of the invention is directed to a pharmaceutical composition comprising, as an active Ingredient, a peptide according to the invention, together with a pharmaceutically acceptable vehicle, and to the use of a peptide according to the invention for the manufacture of a pharmaceutical composition for treating and/or preventing Insulin deficiency in a patient. The vehicle is selected by the manufacturer based on the desired route of administration, and examples of suitable vehicles can be found in the US or European pharmacopoeia.

In yet another highly preferred embodiment, the present invention relates to a novel vasoconstrictor, more precisely to a synthetic peptide derived from the intracellular C-terminus of angiotensin 1A receptor. The peptide is a functional protein-mimicking CPP and promotes contraction of heart coronary blood vessels of different origin.

The CPP related to herein is derived from the ATLA receptor, comprising a peptide corresponding to at least one fragment of the C-terminal tail, comprised in the third and/or second intracellular loop of the receptor.

The disclosed peptide, mimicking agonist-activated ATLA receptor, is of particular interest as potential drug, useful in the situations where vasoconstriction is required, e.g. in chronical hypotension or migraine.

The present invention thus in one embodiment comprises the synthesized peptide M511, derived from the C-terminal intracellular part of the rat AT1A receptor. M511 is able to translocate into a human melanoma cell line Bowes. The effects of M511 and biotinylated M511 were tested with porcine coronary arteries and veins, as well as with human umbilical blood vessels (as can be seen in example 8). In all cases, peptides triggered contraction of blood vessels. The sequence to which the M511 peptide corresponds is conserved within the AT1 receptor subfamily, but has low similarity to AT2 type receptor (see table 2).

TABLE 2

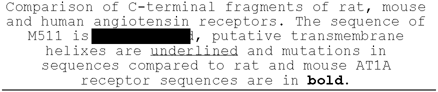

| origin | Segments 291-330 for AT1 and 307-346 for AT2 receptors |
|---|---|
| Rat AT1A | 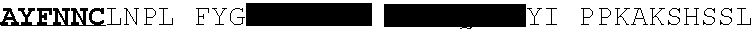 |
| Mouse AT1A | AYFNNCLNPL FYGFLGKKFK KYFLQLLKYI PPKAKSHSSL |
| Human AT1A | AYFNNCLNPL FYGFLGKKFK RYFLQLLKYI PPKAKSHSNL |
| Rat AT1B | AYFNNCLNPL FYGFLGKKFK RYFLQLLKYI PPKARSHAGL |
| Mouse AT1B | AYFNNCLNPL FYGFLGKKFK RYFLQLLKYI PPKARSHAGL |
| Human AT1B | AYFNNCLNPL FYGFLGKKFK KDILQLLKYI PPKAKSHSNL |
| Rat AT2 | GFTNSCVNPF LYCFVGNRFQ QKLRSVFRVP ITWLQGKRET |
| Mouse AT2 | GFTNSCVNPF LYCFVGNRFQ QKLRSVFRVP ITWLQGKRET |
| Human AT2 | GFTNSCVNPF LYCFVGNRFQ QKLRSVFRVP ITWLQGKRES |

Although the inventors have not demonstrated its selectivity to AT1 mediated signal transduction, they show that the peptide activates specifically the same type of G-proteins as agonist activated AT1A receptor. Scrambled M511 (as can be seen in table 3) were prepared and tested as a control, with no effect in blood vessel contraction.

TABLE 3A
Sequences of penetratin, M511 and scrambled M511.

| Name | Sequence |
|---|---|
| Penetratin | RQIKIWFQNRRMKWKK |
| M511 | FLGKKFKKYFLQLLK |
| ScrM511 | KGKFQLYLKLKFKFL |

TABLE 3B
novel analogues of M511 that fullfill the selection criteria according
to the present invention and that might potentially be effective
in inducing long-lasting contraction of blood vessel in a similar
manner as M511, see also SEQ. ID. NO. 31887-31894.
(Cit-citrulline, Fph-4-fluoro-phenylalanine)

| Name | Sequence/Name |
|---|---|
| Analogue 1 | KKFKKYFL |
| Analogue 2 | KKYFLQLLK |
| Analogue 3 | FKKYFLQLL |
| Analogue 4 | KKFKKYFLQ |
| Analogue 5 | Cit-Cit-Phe-Cit-Cit-Fph-Ile |
| Analogue 6 | Cit-Cit-Fph-Ile-Cit-Ile-Ile-Cit |
| Analogue 7 | Phe-Cit-Cit-Fph-Ile-Cit-Ile-Ile |
| Analogue 8 | Cit-Cit-Phe-Cit-Cit-Fph-Ile-Cit |

To determine the biological effects of the above listed M511 analogues, they can easily be tested with porcine coronary arteries and veins, as well as with human umbilical blood vessels (as exemplified in example 8).

Unique properties of the novel peptide M511 are e.g. that it penetrates cell membranes by non-endocytotic mechanism, it induces long-lasting contraction of blood vessel and this contraction is peptide sequence specific. Furthermore, it interacts with G-proteins and mimics agonist activated ATLA receptor.

Thus, one aspect of the invention is directed to a peptide selected from the group consisting of peptides having the amino acid sequence FLGKKFKKYFLQLLK (=M511) and to cell membrane penetrating analogues thereof as well as to non-peptide membrane penetrating analogues.

The cell-penetrating analogues of the peptide M511 (derived from rat ATLA receptor) may be corresponding peptides from other mammalian species or individual variants from the same species, or non-peptide analogues, and may thus have amino acid extensions, deletions or substitutions in relation to the amino acid sequence of the peptide M511, as long as they have cell-penetrating properties.

Analogue to the above synthetic peptide derived from the 1A receptor, yet another vasoconstrictor is comprised in the scope of the present invention, designed and produced by the inventors, which is derived from CGRP receptor loop iC4, sequences 391-405 (VQAILRRNWNQYKIQ) and named M630, see SEQ.ID.NO. 31895. As shown in example 17, it penetrates cell membranes by a non-endocytotic mechanism, induces long-lasting contraction of blood vessel in a peptide sequence specific mode.

Thus, another aspect of the invention is directed to a peptide selected from the group consisting of peptides essentially comprising the amino acid sequence VQAILRRNWNQYKIQ (=M630) and to cell membrane penetrating analogues thereof, which can be found using the selection criteria as disclosed herein.

The cell-penetrating analogues of the peptide M630 may be corresponding peptides from other mammalian species or individual variants from the same species, or non-peptide analogues, and may thus have amino acid extensions, deletions or substitutions in relation to the amino acid sequence of the peptide M630, as long as they display cell-penetrating properties.

In an additional embodiment of the invention, a CPP as described above is coupled to a cargo. The cargo may be a marker molecule, such ds biotin.

Another aspect of the invention is directed to a peptide of the invention for use as a vasoconstrictor, and to its use for the manufacture of a pharmaceutical composition for treating and/or preventing vasoconstriction.

Yet another aspect of the invention is directed to a pharmaceutical composition comprising, as an active ingredient, a peptide according to the invention, together with a pharmaceutically acceptable vehicle. The vehicle is selected by the manufacturer based on the desired route of administration, and examples of suitable vehicles can be found in the US or European pharmacopoeia.

Still another aspect of the invention is directed to a method of inducing contraction of blood vessels in an individual comprising the steps of administering to said individual a vasoconstricting amount of a peptide according to the invention, or a pharmaceutical composition according to the invention.

### Transmembrane-Protein Derived CPPs:

In another, equally preferred embodiment, a CPP related to in the present context can stem from any other transmembrane peptide, and is by no means limited to being derived from a receptor. As disclosed in example 6, one embodiment of the present invention thus relates to a CPP derived from mouse PrpC (1-28): MANLG YWLLA LFVTM WTDVG LCKKR PKP, human PrpC(1-28): MANLG CWMLV LFVAT WSDLG LCKKR PKP, or bovine PrpC (1-30): MVKSK IGSWI LVLFV AMWSD VGLCK KRPKP. See SEQ.ID.NO. 31896-31899.

As disclosed in example 7, even amyloid precursor protein (APP) and presenilin-1 (PS-1) have cell-penetrating sequences and are consequently included as sources for a CPP derived from their amino acid sequence, according to a method described in the present application. See SEQ.ID.NO. 31900-31906.

### Detected Potential CPPs

In the present context, cell-penetrating peptides can be derived or de novo designed from both random peptide sequences, and from naturally occurring proteins. Typical examples for a de novo designed CPP are given in Table 18 bellow and listed as SEQ.ID.NO.31923-31940.

TABLE 18

| | |
|---|---|
| Evo162 | KTVLLRKLLKLLVRKI |
| Evo163 | KIIKRLIVVRLITLVIK |
| Evo164 | LLKLKLLAILKIKLIV |
| Evo83 | KLIRKRLI |
| Evo86 | RLIKRLIK |
| Evo86 dimer | (RLIKRLIKC)₂ |
| Evo165 | LLKKRKVVRLIKFLLK |
| Evo165 analogue | LLKKRKVVRLIKQKQK |
| Evo165 analogue | LLKKRKVRLIKQKQK |
| Evo165 analogue | LLKKRKVVRLIKAHSK |
| Evo165 analogue | LLKKRKVRLIKAHSK |
| Evo165 analogue | LLKKRKVVRLIKVRK |
| L-407-Abz | LKLLYKNKLLKYNLKamide |
| L-408-Abz | KLFKYKKLKRYFYLQKamide |
| L 409-Abz | YKRLSLVKRLIKamide |
| Evo165-B | Biotin-LLKKRKVVRLIKFLLKamide |
| Evo165 | LLKKRKVVRLIKFLLKamide |

Furthermore, a naturally occurring sequence can of course be modified to become cell-penetrating or to be optimised with regards to its cell-penetrating capacity.

A cell-penetrating peptide and/or a non-peptide analogue thereof detected by a method according to the present invention is preferably selected from a 8 to 50 amino acid residues long peptide, such as a 8 to 30 amino acid residues long peptide, or a 14 to 30 amino acid residues long peptide, or a 16 to 20 amino acid residues long peptide.

In special circumstances, though, said cell-penetrating peptide and/or a non-peptide analogue thereof detected by a method according to the present invention can also consist of at least 2, 3, 4, 5, 6, or 7 amino acids.

In a typical embodiment of the invention, a cell-penetrating peptide is selected from a 12 to 50 amino acid residues long peptide or a fragment of a peptide of one of the amino acid sequences as listed in the accompanying sequence listing as SEQ.ID.NO. 1-150.

In another, equally preferred embodiment of the invention, a cell-penetrating peptide is selected from a 8 amino acid residues long peptide or a fragment of a peptide of one of the amino acid sequences as listed in the accompanying sequence listing as SEQ.ID.NO. 6234-7420.

In yet another embodiment of the invention, a cell-penetrating peptide is selected from a 12 amino acid residues long peptide or a fragment of a peptide of one of the amino acid sequences as listed in the accompanying sequence listing as SEQ.ID.NO. 151-2684, and as SEQ.ID.NO. 7421-11649.

Additionally, in a further embodiment of the invention, a cell-penetrating peptide is selected from a 16 amino acid residues long peptide or a fragment of a peptide of one of the amino acid sequences as listed in the accompanying sequence listing as SEQ.ID.NO. 2685-6233, and as SEQ.ID.NO. 11650-18398.

### Cargo

As described previously, a CPP can be coupled to a cargo to function as a carrier of said cargo into cells, various cellular compartments, tissue or organs. The cargo may be selected from the group consisting of any pharmacologically interesting substance, such as a peptide, polypeptide, protein, small molecular substance, drug, mononucleotide, oligonucleotide, polynucleotide, antisense molecule, double stranded as well as single stranded DNA, RNA and/or any artificial or partly artificial nucleic acid, e.g. PNA, a low molecular weight molecule, saccharid, plasmid, antibiotic substance, cytotoxic and/or antiviral agent. Furthermore, the transport of cargo can be useful as a research tool for delivering e.g. tags and markers as well as for changing membrane potentials and/or properties, the cargo may e.g. be a marker molecule, such as biotin.

With respect to the Intended transport of a cargo across the blood-brain barrier, both intracellular and extracellular substances are equally preferred cargo.

Naturally, not every CPP will be equally qualified for transporting any and each cargo, such as has e.g. been shown for Tat and Penetratin, not being optimal for transporting highly negative charged cargo, such as DNA. Thus, the selection of most optimal CPP of choice for transporting a certain cargo will have to be estimated and verified by the person skilled in the art, and will be highly dependent on the nature of the specific cargo and the target cell/tissue.

In a preferred embodiment of the invention, the cell-penetrating peptide is coupled by a S—S bridge to said cargo. Naturally, there are a broad variety of methods for coupling a cargo to a CPP, selected individually depending on the nature of CPP, cargo and intended use. A mode for coupling can be selected from the group consisting of covalent and non-covalent binding, as biotin-avidin binding, ester linkage, amide bond, antibody bindings, etc.

In some embodiments, a labile binding is preferred, in other embodiments, a stabile binding is elementary, such as in the use of a CPP according to the present invention for use in transport of medical substances, due to the necessary storage of said pharmaceutical compositions before use.

Illustrative examples for the above described embodiments are given in examples 4, 6, 14 and 15.

In example 14, a methotrexate (MTX) conjugate with a CPP carrier is described. MTX is a cytotoxic drug, which was developed for the treatment of malignancies but is now also used to treat autoimmune diseases, such as psoriasis. Normally, MTX is present in bodily fluids as negatively charged molecule. Therefore, it can cross the cell membrane only with difficulty.

Thus one Illustrative example of the use of a CPP for the transport of a cargo according to the present invention comprises MTX-CPP conjugates essentially as comprised in SEQ.ID.NO. 31907-31911, which are e.g. synthesised using a solid phase peptide synthesis strategy as described in example 14, specific examples of which are listed in table 16 below:

TABLE 16
MTX-CPP conjugates

Apa-γGlu-Gly-CPP
Apa-(γGlu)2-5-Gly-CPP
Apa-Cys-S-S-Cys-CPP

Another example of a molecule being carried across a cellular membrane with a CPP is given in example 15, wherein siRNA uptake is substantially improved.

In recent years small interfering RNA (siRNA) have gained attention for their highly sensitive ability to regulate gene expression in mammalian cells. siRNA are short strands (about 21-23 bp) of double stranded RNA that induce specific cleavage of their complementary mRNA through activation of the RNA-induced silencing complex (RISC). Although RNA-induced silencing is an endogenous mechanism, synthetically synthesized siRNA's could bee shown to have the same effect both in vitro and in vivo.

A well known problem when using siRNA, is low yield of uptake in the cell. By coupling cell-penetrating peptides (CPP) to synthetically synthesized siRNA though, the cellular uptake is significantly improved.

Another specific embodiment of the use of a CPP for cargo transport included in the present invention thus relates to a sIRNA against the GALR-1 mRNA coupled to a CPP, such as Transportan10 (Tp10) via a disulfide linker, e.g. as shown in FIG. 36 and listed as SEQ ID NO. 31912.

### Improvement of a Known Cellular Penetration Method

Additionally, a CPP discovered by a method according to the present invention can also be used for the improvement of a known cellular penetration method, such as for the improvement of gene delivery in vivo, comprising transfection, microinjection, transduction or electroporation.

During the past 40 years, DNA delivery, especially via the nonviral route (i.e., transfection), has become a powerful research tool for elucidating gene function and regulation. Nonviral gene delivery systems generally exhibit a superior safety compared to viruses, which are more commonly used especially in clinical trials, however, their relatively low efficiency of transgene expression is a major obstacle (Ma, H. & Diamond, S. L. Nonviral gene therapy and its delivery systems. Curr Parm Biotechnol 2, 1-17. (2001)). The efficiency of DNA delivery is dependent on several steps: adsorption of transfection complex to the cellular surface, uptake by the cell, endosomal release, nuclear translocation and expression of the gene.

### Nonviral Transfection Methods

### Nonviral Transfection Reagents Available Today are Mainly Working in Three Ways:

Increasing the uptake of the plasmid across the plasma membrane, destabilizing the endosomal membrane and enhancing nuclear uptake. The main transfection protocols and reagents include: 1) calcium phosphate precipitation; 2) cationic polymers as DEAE dextran, polylysine and polyethyleneimine (PEI) (Garnett, M. C. Gene-delivery systems using cationic polymers. Crit. Rev Ther Drug Carrier Syst 16, 147-207 (1999)); 3) physical methods like microinjection and electroporation, DNA gun and similar (Somiari, S. et al. Theory and in vivo application of electroporative gene delivery. Mol Ther 2, 178-87. (2000)); and 4) liposomal vectors like cationic and anionic liposomes (Lee, R. I. & Huang, L. Lipidic vector systems for gene transfer. Crit. Rev. Therap. Drug Carrier Syst. 14, 173-206 (1997)). Many of these methods and transfection reagents are working well in vitro and for ex vivo transfections but are less suitable for in vivo gene transfer (Ma, H. & Diamond, S. L. Nonviral gene therapy and its delivery systems. Curr Pharm Biotechnol 2, 1-17. (2001)). In general, methods with high delivery efficiency are also toxic for the cells. One exception is microinjection, which is both effective in delivery and non-toxic, but unfortunately can not be used en masse (Luo, D. & Saltzman, W. M. Enhancement of transfection by physical concentration of DNA at the cell surface. Nat Blotechnol 18, 893-5). Old chemical reagents and methods like DEAE-dextran and calcium phosphate precipitation are simple, effective and still widely used but both suffer of cytotoxicity and are difficult to apply in vivo. Lipofection lacks cell specific targeting and the structure of DNA-lipid complexes are poorly understood.

For introduction of DNA into cells, favourite methods have been complexing with different compounds. This approach allows easy preparation of transforming agent and therefore quick modification of DNA construct and transformation conditions. Primary role of complexing agents is neutralization of the negative charge of phosphate groups in the DNA backbone and condensing the large DNA molecule. An average DNA molecule used for delivery of foreign DNA has to be at least 3000 base pair long to be propagated in bacterial cells during preparation. Most of modern plasmids for mammalian cell expression are 4500 to 5000 bp long i.e. have Mw over 3,000,000. After neutralization of negative charge and packing into tight particles DNA molecules are taken up by cells via endocytotic pathways. Classical transfection methods/agents have been modified in many ways attempting to prevent or neutralize degradation pathway activation in response to endocytosis.

Ca-phosphate transfection method remains still the most popular and widely used. The main reason for the popularity is very low cost. However, the method is extremely cell type specific and toxic for many cell types including neuronal and primary cells. Many attempts have been made to include DNA into liposome-like structures. Other methods relay on complexing of DNA with polymeric molecules that bind to DNA. A major problem with all those approaches have been toxicity to cells.

### Polyplex Technique

Another approach that has gained a lot of prominence in last years, is the use of transfection systems based on the principle of condensing DNA with polycations. According to renewed nomenclature, this technique is referred to as polyplex (Felgner, P. L. et al. Nomenclature for synthetic gene delivery systems. Hum Gene Ther 8, 511-2. (1997)). Polyplexes are more effective than lipid based vectors and also, in most cases, less toxic (Gebhart, C. L. & Kabanov, A. V. Evaluation of polyplexes as gene transfer agents. J Control Release 73, 401-16. (2001)). One of the cheapest, very effective and most widely used polycation is polyethylenelmine (PEI) (Boussif, O. et al. A versatile vector for gene and oligonucleotide transfer into cells in culture and In vivo: polyethylenimine. Proc Natl Acad Sci U S A 92, 7297-301. (1995), Abdallah, B. et al. A powerful nonviral vector for in vivo gene transfer into the adult mammalian brain: polyethylenimine. Hum Gene Ther 7, 1947-54. (1996), Schatzlein, A. G. Non-viral vectors in cancer gene therapy: principles and progress. Anticancer Drugs 12, 275-304. (2001)). Since it has a high positive net charge, it neutralises negative charges of dsDNA and also condenses DNA. Compact PEI/DNA globules Internalise into cells mostly by endocytosis (Remy-Kristensen, A., Clamme, J. P., Vuilleumier, C., Kuhry, I. G. & Mely, Y. Role of endocytosis in the transfection of L929 fibroblasts by polyethylenimine/DNA complexes. Biochim Blophys Acta 1514, 21-32. (2001)). These complexes also promote transfection by preventing degradation of DNA by lysosomal enzymes and by enhancing the release of DNA from the endocytic vesicles (Gebhart and Kabanov 2001). Unfortunately, though, at concentrations successfully used in vitro, the polycations are still too toxic for systemic in vivo use.

The approach described in the present application discloses a principally new way of transporting large DNA molecules across a cell membrane. Instead of relying on cell-activity-dependent endocytosis, an active transport of plasmids is proposed, using the capacity of cell-penetrating peptides (CPP) to carry cargoes Into cells. While CPPs are performing the transport action, a second component neutralises phosphate groups and condenses (packs) the large DNA molecule. Further, packing agents that mask the phosphates have always had an additional function as proton buffers. Binding of protons neutralises lysosomes and Inhibits many degradation pathway enzymes. Also, escaping of the degradation pathways by active transport over the cell membrane dramatically reduces the amount of phosphate neutralising/packing agent necessary, and therefore lowers its toxic effects.

As described in example 9, the invention thus also relates to an improved polyplex mediated gene delivery method, wherein a cell-penetrating peptide and/or a non-peptide analogue thereof. Is conjugated either to a reporter gene or to a transfection reagent, such as e.g. polyethylene amine. In both cases enhanced expression of reporter proteins, GFP and luciferase, are observed.

In this study the inventors developed a new gene delivery system. By combining the effects of PEI and TP10 or YTA-2 together, substantially higher transfection ratios were achieved than with PEI only. The approach was to crosslink TP10 or YTA-2 to transfection reagent. Thereafter PEI of common transfection protocol was replaced by CPP modified one. Under optimal conditions, the results postulate a significant improvement in gene delivery compared to other systems.

TABLE 4
Comparison of transfection methods with PEI-TP10/YTA-2
methods (+++ being good transfection efficiency).

| | Transfection | Toxicity | | |
|---|---|---|---|---|
| Method | efficiency | in vitro | in vivo | Remarks |
| Viruses | | | | |
| adenovirus | +++ | low | variable | immunogenic |
| Retrovirus | +++ | low | low | revertant risk |
| Lentiviruses | +++ | low | unknown | |
| Microinjection | +++ | low | not | |
| | | | applicable | |
| Electroporation | ++(+) | high | high | |
| Ca₃(PO₄)₂ | ++ | high | high | |
| precipitation | | | | |
| Lipofection | | | | |
| cationic | ++ | medium | high | |
| liposomes | | | | |
| anionic | +(+) | medium | high | |
| liposomes | | | | |
| Polycations | | | | |
| DEAE-dextran | +(+) | medium | medium | |
| Polylysine, | +(+) | low | medium | |
| Polyornithine | | | | |
| Loligomers | +(+) | medium | medium | |
| dendrimers | ++ | medium | medium | |
| polyethyleneimine | ++ | medium | medium | |
| Trasferrin-PEI | ++ | low | medium | cell selective |
| Polyethylene | ++ | low | medium | cell selective |
| glycosylated | | | | |
| PEI | | | | |
| PNA-NLS + PEI | ++(+) | low | unknown | low PEI doses |
| present | ++(+) | low | low | low PEI doses |
| method | | | | |

Consequently, the present invention in particular relates to a vector for (non-viral) cell transfection, comprising a) a nucleic acid component, b) a polycation conjugate, and c) a cell-penetrating peptide and/or a non-peptide analogue thereof, such as YTA-2 (SEQ.ID.NO. 31913), which is able to enhance the average rate of transfection per cell at identical transfection conditions by a factor of at least 2, such as by a factor of at least 5, 10, or 15, compared to a vector comprising only components a) and b), or only a) and c).

Also envisioned herein is a vector as described above, for usage in a transient transfection and/or a stable transfection of a cell in vivo and/or in vitro, for transfecting a mammalian cell such as a cell selected from the group consisting of human, rodent, pig, cow.

A vector as described above will typically comprise DNA as oligonucleotide and/or polynucleotide and said polycation conjugate will be polyethyleneimine (PEI), polyornithine, polylysine, polyamines, dendrimers, spermidine, DEAE-dextran, patricine, transferrin-PEI, polyethylene glycosylated PEI, or loligomers.

Consequently, the present invention also relates to a method for in vivo transfecting a cell in a host tissue with a nucleic acid, comprising introducing a vector according to the present invention, e.g. as illustrated in example 9, for in vivo transfecting a cell in a host tissue and/or an isolated cell with a nucleic acid.

### Cell-Selective CPPs

In yet a further embodiment of the present invention, a cell-penetrating peptide and/or a non-peptide analogue thereof is provided that will enter selectively into a certain cell type/tissue/organ, or that transports a cargo that will only be activated in a certain cell type, tissue, or organ type.

The inventors show that different CPPs are internalised by specific cell lines, such as human melanoma cell line Bowes and others, with significantly different efficacy and rate of uptake, sometimes more than two-fold. This is a prerequisite to define CPPs that are internalised with different efficacy to different cell lines and tissues. Hence, an Important embodiment of the present invention is a method for the development of selective CPPs (selCPPs) characterised e.g. by testing all available natural or de novo designed CPPs for cellular uptake in cell lines, cells, tissues and/or organs into which a selective transport is required. On the other hand, certain selective methods might be employed to artificially enhance the cell selectivity of a CPP of choice for a certain target cell or target cell population, which will be described in detail below.

As an example, cancer cells expose many cell surface antigens and/or proteins, as well as secrete certain proteins. Usually, tumour cells do not express cell surface markers that are unique but rather over-express common receptors/markers. The signalling through these over-expressed markers and/or over-amplifications of the intracellular and/or extracellular signals is thought to be one of the mechanisms for the loss of control of the cellular machinery over the cell cycle. Thus, in a specific embodiment of the present invention, an over-expressed cell surface protein and/or secreted protein is applied as target for CPP addressing.

In one embodiment of the invention, a cell-selective CPP (selCPP) is envisioned that comprises an antigen/protein raised against a cellular marker, selected from the group consisting of channel receptors, tyrosine kinase receptors (e.g. EGF, IGF), guanylate cyclase receptors, serine/threonine kinase receptors, cytokine receptors, receptors coupled to guanosine triphosphate (GTP)-binding proteins (G protein-coupled receptors: GPCRS), glycosphingolipids, CD44, neuropeptide receptors, e.g. neurotensin receptors, galanin, and substance P receptors.

Generally, a cell-selective CPP (selCPP) will of course be extremely useful in the targeted transport of any kind of drug or pharmaceutical substance to a variety of specific eukaryotic and/or prokaryotic cellular targets. A cell-selective transport of such cargo is e.g. envisioned for an improved treatment or prevention of infectious diseases, such as diseases caused by a viral, bacterial or parasital infection.

In order to avoid non-specific internalisation of CPPs before finding a target cell in vivo, the invention in a further aspect relates to a new variation of the enzyme-prodrug strategy, wherein a selCPP-conjugate is designed so that the cell penetration-active structure of the CPPs is disrupted until the binding event of a peptide part of said selCPP to a cell/tissue or organ specific receptor/marker, or the cleavage of said selCPP-conjugate by a protease secreted by the target cell/tissue or organ, releases the CPP from conformational discrimination.

The term “enzyme-prodrug strategy/therapy” is in the field of the art used to define a specific approach to delivering a drug, which is focused on the development of amino-acid or nucleic-acid prodrugs, which, before or after delivery, require activation by more or less tissue, organ and/or cell-selective enzymes. Large differences in selectivity are found in the prior art. For some prodrugs, a rapid removal of the released drug from the target tissue/organ/cell explains the low selectivity, whereas for others, cleavage in non-target tissue and Insufficient transport across the cell to the enzyme site seems mainly responsible.

Especially many anticancer agents are severely toxic, which explains the need of more effective and less toxic prodrugs and/or softdrugs. A typical prodrug/softdrug must therefore be an efficient and selective substrate for the activating enzyme, and be metabolised to a potent cytotoxin and/or cytostatica, which is preferably able to kill cells at all stages of the cell cycle. Many of the early antimetabollte-based prodrugs provided very polar activated forms that had limited abilities to diffuse across cell membranes, and relied on gap junctions between cells for their bystander effects. Prodrugs as described in the present invention, though, have good distributive properties and their activated species are naturally cell penetrating, so that the resulting bystander effects can maximize the effectiveness of the therapy.

In the present context, the term “enzyme-prodrug strategy/therapy” is additionally used to describe the above revealed method of delivering a drug, wherein the drug itself or Its transporter CPP is rendered non-cell-penetrating in order to avoid non-specific Internalisation of CPPs before finding a target cell in vivo, and wherein only the binding event of a peptide part of said selCPP to a cell/tissue or organ specific receptor/marker, or the cleavage of said selCPP-conjugate by a protease secreted by the target cell/tissue or organ, releases the CPP from conformational discrimination, whereupon it can penetrate the target cell.

Cell type targeted CPPs can further be modified by a non-covalent intermolecular interaction with the part of a receptor targeting sequence. After binding to the receptor, the CPP is herein displaced by a receptor and the CPP will internalise, see FIG. 1 and FIG. 2. Receptor internalisation is relatively slow, as compared to the CPP translocation.

In another, equally preferred aspect of the present invention, the selCPP is in contrast to the above, selected or designed particularly not to be cell penetrating. Strictly spoken, such a selCPP should be called a “sel-non-CPP”. The intention being that the non-CPP is released not into the target cell, but after coupling to the specific cell, is released into the surrounding extracellular space. In principal, the selection criteria described in the present application can of course as well be used to predict, verify, design and/or produce a peptide that is not cell penetrating. Said non-CPP should then be characterised by not having a Z_{Σ} bulk property value essentially consisting of individual average interval values, wherein most preferably Z_{Σ1}<0.2; Z_{Σ2}<1.1; Z_{Σ3}<−0.49; Z_{Σ4}<0.33; and Z_{Σ5}<1.1 and Z_{Σ5}>0.12. Neither should it in alternative embodiments of the invention, have individual average values that comprise Z_{Σ1}<0.3, such as Z_{Σ1}<0.21, Z_{Σ1}<0.22, Z_{Σ1}<0.23, Z_{Σ1}<0.24,

### Z_{Σ1}<0.25, Z_{Σ1}<0.26, Z_{Σ1}<0.27, Z_{Σ1}<0.28, or Z_{Σ1}<0.29;

Z_{Σ2}<1.2, such as Z_{Σ2}<1.11, Z_{Σ2}<1.12, Z_{Σ2}<1.13, Z_{Σ2}<1.14, Z_{Σ2}<1.15, Z_{Σ2}<1.16, Z_{Σ2}<1.17, Z_{Σ2}<1.18, or Z_{Σ2}<1.19;
Z_{Σ3}<−0.39, such as Z_{Σ3}<−0.4, Z_{Σ3}<−0.41, Z_{Σ3}<−0.42, Z_{Σ3}<−0.43, Z_{Σ3}<−0.45, Z_{Σ3}<−0.46, Z_{Σ3}<−0.47, or Z_{Σ3}<−0.48;
Z_{Σ4}<0.43, such as Z_{Σ4}<0.34, Z_{Σ4}<0.35, Z_{Σ4}<0.36, Z_{Σ4}<0.37, Z_{Σ4}<0.38, Z_{Σ4}<0.39,

### Z_{Σ4}<0.4, Z_{Σ4}<0.41, or Z_{Σ4}<0.42;

Z_{Σ5}<1.05 and Z_{Σ5}>0.22, such as Z_{Σ5}<1.04 and Z_{Σ5}>0.21, Z_{Σ5}<1.03 and Z_{Σ5}>0.20, Z_{Σ5}<1.02 and Z_{Σ5}>0.19, Z_{Σ5}<1.01 and Z_{Σ5}>0.18, Z_{Σ5}<1.00 and Z_{Σ5}>0.17, Z_{Σ5}<0.99 and

### Z_{Σ5}>0.16, Z_{Σ5}<0.98 and Z_{Σ5}>0.15, Z_{Σ5}<0.97 and Z_{Σ5}>0.14, or Z_{Σ5}<0.96 and Z_{Σ5}>0.13.

An especially preferred embodiment of the present invention thus relates to a cell-selective delivery system for a cytostatic and/or cytotoxic agent, comprising a) a protease consensus site for a protease specifically overexpressed in a target cell, b) a cell penetrating peptide and/or a non-peptide analogue thereof, and c) a cytostatic and/or cytotoxic agent, wherein said cell-selective delivery system additionally comprises an inactivation sequence repressing the cellular penetration capacity of said cell-penetrating peptide, and which is cleaved by said protease specifically overexpressed in the target cell upon introducing said cell-selective delivery system in the near vicinity of said target cell. See e.g. example 12.

A typical example for the above concept are matrix metallo proteases (MMPs), which are Zn²⁺ metallo endopeptidases. The family contains both membrane bound and secreted members of which both catalyse the breakdown of proteins located either on the cell's plasma membrane or within the extracellular matrix (ECM) (M. D. Stemlicht and Z. Werb How matrix metallo proteinases regulate cell behavior” Annual Review of Cell and Developmental Biology, 17:463-516, 2001). MMPs have been linked to the invasive and metastatic behaviour of a wide variety of malignancies, and these enzymes are generally overexpressed in a variety of tumours (M. D. Sternlicht and Z. Werb “How matrix metallo proteinases regulate cell behavior” Annual Review of Cell and Developmental Biology, 17:463-516; D. V. Rozanov et al., “Mutation analysis of membrane type-1 metalloproteinase (MT1-MMP, alternative name MMP-14)”, Journal of Biological Chemistry (IBC), 276:25705-14, Jul. 13, 2001). Membrane type MMPs (MT-MMP), such as MMP-MT1 have been strongly implicated in oncogenesis. These enzymes localise to the invasive fronts. The soluble MMPs 1-3 and 9 have also been implicated as agonists of tumourigenesis (Smith, L. E., Parks, K. K., Hasegawa, L. S., Eastmond, D. A. & Grosovsky, A. J. Targeted breakage of paracentromeric heterochromatin induces chromosomal instability. Mutagenesis 13, 435-43. (1998)).

Concomitantly, and as elegantly proven in examples 4 and 12, the present invention relates to a method for designing a selCPP, based on three basic functions: 1) selective cleavage (and thereby activation) by MMP-2 or MMP-MT1, 2) cellular penetration by peptides (CPPs) and 3) killing of nearby, preferably tumour cells or endothelia involved in tumour neovascularisation, by a known cytostatic and/or cytotoxic agent (see FIGS. 3, 4, 25 and 26).

The present invention thus comprises a selCPP selected from an amino acid sequence contained in table 5 or table 6, and to a combined cell-selective delivery system for a cytostatic and/or cytotoxic agent, comprising an amino acid sequence listed in table 5 or table 6, and a cytostatic and/or cytotoxic agent. See SEQ.ID.NO.31913-31922.

TABLE 5
selCPPs based on MMP-2 (gelatinase-A) cleavage specificity:

| Name | Sequence | MMP site | Penetration | Comment: |
|---|---|---|---|---|
| YTA-2 | YTAIAWVKAFIRKLRK | SGESLAY-YTA | +++ | stain also nuclear |
| | | | | membrane FIG. 3 |
| YTA-2ps* | SGESLAY- | SGESLAY-YTA | + | bind the plasma |
| | YTAIAWVKAFIRKLRK | | | membrane FIG. 4 |

*ps stands for proteinase cleavage site

TABLE 6
selCPPs based on MMP-MT1 cleavage specificity

| Name | Sequence | MMP site |
|---|---|---|
| LRSW-1 | LRSWVISRSIRKAA | GPLG-LRSW |
| LRSW-2 | LRSWIRRLIKAWKS | GPLG-LRSW |
| LRSW-3 | LRSWRVIIRNGQR | GPLG-LRSW |

Consequently, the present invention also relates to a cell-selective delivery system for a cytostatic and/or cytotoxic agent, comprising a) a cell-penetrating peptide and/or a non-peptide analogue thereof comprising a protease consensus site for a protease specifically overexpressed in a target cell and c) a cytostatic and/or cytotoxic agent, wherein said cell-selective delivery system additionally comprises an Inactivation sequence repressing the activity of said cell-penetrating peptide, and which is cleaved by said protease specifically overexpressed in the target cell upon introducing said cell-selective delivery system in the near vicinity of said target cell.

In a preferred embodiment of said cell-selective delivery system, as described above, said cell-penetrating peptide comprised in said cell-selective delivery system enhances the average rate of cellular uptake of said cytostatic and/or cytotoxic agent into said selective cell per cell by a factor of at least 1.5 compared to the average rate of cellular uptake into said cell of a cell-selective delivery system comprising only components a) and c), or to the average rate of cellular uptake of component c) alone of said cell.

In another, equally preferred embodiment of said cell-selective delivery system, as described above, said cell-penetrating peptide comprised in said cell-selective delivery system enhances the average rate of cellular uptake of said cytostatic and/or cytotoxic agent into said selective cell per cell by a factor of at least 1.5, such as at least 2, 3, 4, 5, 6, 7, 8, 9, 10, 15, 20, 25, 30, 35, 40, 50, 60, 70, 80, 90, 100, 1000 or 10 000, compared to the average rate of cellular uptake into said cell of a cell-selective delivery system comprising only components a) and c), or to the average rate of cellular uptake of component c) alone of said cell.

In another equally preferred embodiment of said cell-selective delivery system as described above, said overexpressed protease is a Zn²⁺ metallo endopeptidase selected from the group consisting of MMP-1, MMP-2, and MMP-MT1.

In yet another embodiment, said overexpressed protease is selected from the group consisting of bacterial surface proteases and viral enzymes.

Furthermore enclosed in the scope of the present invention is a method for targeting CPPs to particular cells and tissues, i.e. to design and apply selCPPs. A suitable CPP is herein designed with a unique surface marker, which is specific for the designated cell or tissue type, characterised by selecting a CPP from any suitable CPP sequence that is designed by the prediction/selection criteria or found in any other way, selecting peptide X, or address, as an epitope from the suitable cell surface receptor against which a specific monoclonal antibody has been raised against and which recognizes this particular epitope with high affinity, choosing a linker among polypeptides (Glyₙ, Proₙ, GABAₙ, Ahaₙ, etc.), or suitable organic substances in order to achieve required interactions between Peptide X/antibody and CPP/plasma membrane.

Enclosed in the present application is also an in vivo and/or in vitro method for stopping cellular proliferation of a specific cellular population and the use of a cell-selective delivery system as described herein for in vivo and/or in vitro stopping cellular proliferation of a specific cellular population.

A cell-selective delivery system as described above can of course be used for the manufacture of a pharmaceutical composition for stopping cellular proliferation of a specific cellular population in a mammal and for treating a patient suffering from a medical condition characterised by uncontrolled cellular growth, such as any oncological disorder or disease, or immunological and/or metabolic hyperfunction.

A general aspect of the present invention comprises the use of a cell-selective delivery system, or CPP related to in the present invention for the manufacture of a pharmaceutical composition for gene therapy and to the pharmaceutical composition comprising said cell-selective delivery system, or CPP.

Another aspect of the invention is directed to a composition comprising a cell-penetrating peptide and/or a non-peptide analogue thereof, or cell-selective delivery system according to the invention or resulting from performing any one of the methods according to the invention, and a compound selected from peptides, oligonucleotides and proteins that are general modifiers of intracellular metabolic or signalling mechanisms, either Inhibiting or activating.

Yet another aspect of the invention is directed to the use of a cell-penetrating peptide and/or a non-peptide analogue thereof, or cell-selective delivery system according to the invention or resulting from performing any one of the methods according to the invention, for the manufacture of a medicament.

A further aspect of the invention is directed to the use of a composition according to the invention for the manufacture of a medicament.

The different aspects and embodiments of the invention will now be illustrated by the following examples. It should be understood that the invention is not limited to any specifically mentioned details.

### ABBREVIATIONS

Aβ beta-amyloid
AD Alzhelmer's disease
APP Amyloid precursor protein
AT1 angiotensin receptor type AT1
ATLA angiotensin receptor subtype ATLA
AT1B angiotensin receptor subtype AT1B
AT2 angiotensin receptor type AT2
BACE β-site APP-cleaving enzyme
Bio biotin, blotinylated
BSA bovine serum albumin
CPP cell-penetrating peptide
CTF C-terminal fragment
DCC N,N′-dicyclohexylcarbodlimide
DCM dichloromethane
DIEA diisopropylethylamine
DMF dimethylformamide
DNP dinitrophenyl
EOFAD early onset Alzheimer's disease
FITC 5-fluorescein isothiocyanate
Fmoc 9-fluorenylmethoxycarbonyl
GOP IVIAKLKA-amide
GPCR G-protein coupled receptor
GTP guanosine 5′-triphosphate
GTPase guanosine triphosphatase
GTPγS guanosine γ-S-5′-triphosphate
HKR Hepes-Krebbs-Ringer
HOBt N-hydroxybenzotriazole
HPLC high performance liquid chromatography
IDE insulin degrading enzyme
LOAD late onset Alzhelmer's disease
MBHA 4-methylbenzhydrylamine
NFT neurofibrillary tangles
NICD notch intracellular domain
NMP N-methylpyrrolidone
NTF N-terminal fragment
PAF paraformaldehyde
PBS phosphate buffered saline
PNA peptide nucleic acid
PS presenilin
RNA ribonucleic acid
SAPP secretory APP
TACE tumour necrosis factor alpha converting eiz-yim-e
t-Boc tert-butyloxycarbonyl
TBTU 2-(1H-benzotriazole-1-yl)-1,1,3,3-tetramethyluronium tetrafluoroborate
TFA trifluoroacetic acid
TFA trifluoroacetic acid
TFMSA trifluoromethanesulphonic acid

### LEGENDS TO FIGURES

FIG. 1. Scheme of addressing selCPP by application of Interaction with single transmembrane (A) or 7-transmembrane (B) receptor.

FIG. 2. Intramolecularly constrained selCPP looses its constrain upon recognition event by specific receptor and the internalisation takes place.

FIG. 3. Schematic structure of chimeric selCPP.

FIG. 4. Incubation of non-covalent selCPP-AB complex with the selected cells exposing the epitope sequence. Peptide X leads to competitive interaction of AB with the Peptide X sequence in the cell surface protein.

FIG. 5. Example of inactivated selCPP. Internalisation of YTA-2 (A) compared to YTA-2 ps (B) In LoVo cells, both biotynilated peptides detected by TRITC-avidin at 37° C.

FIG. 6. Example of protease activated selCPP, detection by fluorophore/quencher system. Method of determining the specific cleavage of YTA-2 ps of the matrix metallo proteinase-2 (MMP-2).

FIG. 7. Schematic structure of 7™ spanning G-protein coupled receptor and trimeric G-protein.

FIG. 8. Cellular uptake of GOP (M569) in Bowes cells at 37° C., demonstrating both cytosolic and plasma membrane localisation.

FIG. 9. Stimulation of insulin release in rat pancreatic islets by cell-penetrating peptide GOP

FIG. 10. Blood glucose concentrations in healthy rats after i.p. injection of GOP (100 nmol/kg) and glucose (1 g/kg)

FIG. 11. Plasma Insulin levels in healthy rats after i.p. injection of GOP (100 nmol/kg) and glucose (1 g/kg)

FIG. 12. Internalisation of Prpc-avidin-FrrC. 100 fold dilution of avidin-FITC together with 2.5 FM was incubated with Bowes melanoma cells for 3 h. The plasma and nuclear membrane is clearly outlined.

FIG. 13. Translocation of 10 μM peptide at 37° C. in LoVo cells (human colon cancer).

FIG. 14. Cellular Internalisation In N2A cells at a concentration of 10 μM A and B) Cells treated with APP 521-536 coupled to fluorescein. C) Cells treated with APP 725-740 coupled to fluorescein. D) Cells treated with PS-1 97-109 deletion analogue coupled to fluorescein. E) Cells treated with the known CPP penetratin coupled to fluorescein.

FIG. 15. Localization of biotinyl-M511 (A), blotinyl-scrambled M511 (B), natural background (C) and penetratin (D) in Bowes melanoma cells. Concentration of the peptides was 10 μM, Incubation time 1 h at 4° C. Staining was done using 150 nM streptavidin-FITC.

FIG. 16. Effect of M511 (upper and middle panel) and scrambled M511 (lower panel) on the contraction of the porcine left anterior descending coronary artery without epithelium (upper panel) and in the presence of Intact epithelium (middle and lower panel). Contraction force is given in the arbitrary units. The meaning of the arrows: 1-administration of 30 mM KCl; 2—administration of substance P; 3-washing-out; 4-administration of 16 μM M511 or scrambled M511.

FIG. 17. Dependence of maximal observed relative contraction of porcine coronary artery on the concentration of M511 (▪) and blotin coupled M511 (□). Contraction obtained by 90 mM KCl was taken as 100%. Curves were obtained by nonlinear regression procedure using Prism computer package (GraphPad Software Inc., USA), according to the dose-response equation with variable slope (Hill coefficient) but fixed bottom (0%) and top (100%) values.

FIG. 18. Effect of M511 (▪—no preincubation of membranes with peptide; —25 min. preincubation of the membranes with peptide), blotinylated M511 (◯—no preincubation of membranes with peptide), and scrambled M511 (×—no preincubation of membranes with peptide) on the rate of GTPγS binding to the membranes prepared from the porcine left anterior descending coronary artery.

FIG. 19. Effect of phospholipase C inhibitor U73122 on the contraction of the porcine left anterior descending coronary artery induced by M511. Contraction force is given in the arbitrary units. The meaning of the arrows: 1—administration of 30 mM KCl; 2—administration of substance P; 3—washing-out; 4—administration of 16 μM M511; 5—administration of 30 μM U73122.

FIG. 20. Effect of Sar¹-Thr⁸-anglotensin II on the contraction of the porcine left anterior descending coronary artery induced by M511 (upper graph) and anglotensin II (lower graph). Contraction force is given in the arbitrary units. The meaning of the arrows: 1—administration of 30 mM KCl; 2—washing-out; 3—administration of 100 μM M511; 4—administration of 1 μM anglotensin II; 5—administration of 45 μM of Sar¹-Thr⁸-anglotensin II.

FIG. 21A. GFP expression in N2A cells 3 days after transfection with unmodified 60 kDa PEI (A, B and C), or with TP10 modified (D, E and F). Concentration of plasmide was 0.5 μg/well each time; N/P ratio is 4 (A and D), 8 (B and E) or 16 (C and F).

FIG. 21B. GFP activities measured on murine fibroblasts C3H 10T1/2. Concentration of plasmid in each case 1.2 μg/well. expression measured 24 h after transfection. B) luciferase activity shown relatively to control to background. Concentration of plasmid 0.5 μg/well, protein expression measured 72 h after transfection.

FIG. 22. Transfection of mouse neuroblastoma N2A cells with unmodified polyethylenimine (PEI) or chemically crosslinked PEI and cycteinyl YTA2 or TP10 peptide. Two different concentrations of PEI or peptidyl-PEI were tested: 1 or 0.5 μg per 1 ml of media. Transfection was performed for 3 h in serum-free growth media (DMEM) and photos taken 48 h after transfection. Improvement of transfection is registered with application of both, YTA2 and TP10 peptides. YTA2-PEI and PEI-TP10 transfect 2 times more cells than unmodified

PEI, and also the expression level in transfected cells is higher.

FIG. 23. Positional scanning of CPP within human 7Tm receptors. Human 7Tm receptor sequences was downloaded from the swissprot/trmbl databases. The sequences was searched for CPPs of the indicated length (8,12 and 17 aa long). The position of the start of the CPP in the protein is divided by the total length of the protein, is plotted against the frequency of occurrence (fraction of CPPs). Here, a search window size of 17 aa produced the most hits. The four peaks in evidence corresponds the four intracellular parts of the 7Tms, with the largest corresponding the IC3. It can be noted that the CPP functionality seems to correlate well, both with the topology of the 7tms, as well as the proposed G protein activation sites.

FIG. 24. Another example of an Inactivated selCPP. PenMMP14 uptake in Bowes (A) and B)) and Caco-2 (C) and D)). The left column is uptake of fluorescein labelled peptide (A) and C)) and the left with coumarine label (B) and D)).

FIG. 25. Scheme of selCPP activation by matix metalloproteases (a) representative for any tissue/organ/cell specific protease, leading to tissue-(tumour) selective uptake (b).

FIG. 26. Scheme of selCPP activation by matix metalloproteases(a) leading to tissue-(tumour) selective uptake (b).

FIG. 27. Internalization of blotinylated YTA-2 in human colon adenocarcinoma, LoVo cells.A) Detected with streptavidin-FITC B) comparison nuclear staining with Hoechst. B)

FIG. 28. Internalisation of YTA-2 (A) compared to YTA-2 ps (B) In LoVo cells, both biotynilated peptides detected by TRITC-avidin at 37° C. C) and D) YTA-2 in Caco-2 cells detected with streptavidin TRITC and nuclear stain.

FIG. 29. Uptake of fluorescently labeled peptide (F). Figures are shown in % uptake of added peptide.

FIG. 30. Peptide induced luminescence. A., C. The N293 and the C283 cell lines subjected to a 4×8 h interval exposure of PS-1 (7) and PS-1 (11) at concentrations indicated. B., D. The N293 and the C293 cell line after a 4 h peptide exposure.

FIG. 31. Penetration of Apa-γGlu-Gly-Evo165 into cultured human epidermal keratinocytes, immunofluorescent detection.

FIG. 32. Effects of different MTX-PVEC conjugates on Bowes cell viability (assayed using Cell-TiterGlo™). Exposure for 24 h in 10% FBS-MEM.

FIG. 33. Effect of Apa-γGlu-Gly-Evo165 on Bowes cell viability (assayed using Cell TiterGlo™).

FIG. 34. Effects of MTX, Apa-γGlu-Gly-YTA2 and YTA2 on Bowes cell viability (assayed using Cell-TiterGlo™).

FIG. 35. Effects of MTX, Apa-γGlu-Gly-YTA2 and YTA2 on K562 cell viability (assayed using Cell-TiterGlo™). Exposure for 2 days in 7.50% FBS-RPMI.

FIG. 36. siRNA linked with disulfide bonds to Tp10-PNA, schematic view of construct and theoretical mechanism of action in the cell.

FIG. 37. Uptake of 1 μM peptide-DNA construct, 30 min 37° C. in Bowes cells, 24 well plate.

FIG. 38. Example of cellular penetration by well studied CPPs. Peptide internalisation in live Caco-2 cells of 1 μM Fluo-peptide.

FIG. 39. Protein internalisation by YTA-2 of Fluo-Streptavidin 37° C. and T/E, showing the delivery property of a selCPP.

FIG. 40. Protein internalisation by YTA-2 of Fluo-Streptavidin 4° C. and T/E, showing the delivery property and temperature independency of a selCPP.

FIG. 41. Effect of M630 (50 μM) on porcine coronary artery. Arrows indicate the following procedures: 1 application of KCl; 2 washing-out; 3 application of 50 μM M630.

FIG. 42. Cellular internalisation of M630 conjugated to fluorescein in N2a cells, visualised by confocal microscope. The cells were incubated for 1 h at 37° C. with a final peptide concentration of 5 μM.

### EXPERIMENTAL SECTION

### Example 1

### Prediction of Cellular Penetration Properties of a Peptide

### Introduction

In most peptide quantitative structure activity relationship studies (QSAR), a set of dimensionless values is used to describe a composite of the physical characteristics of the amino acids (descriptors). In the classical literature 3 values, Z₁, Z₂ and Z₃ are used for this purpose. Recently Wold and colleagues expanded this descriptor set with 2 more: Z₄ and Z₅; and produced descriptor scales covering 87 natural and non-natural amino acids.

### Method

Using the expanded descriptor scales, bulk property values Zy where assembled for 4 cell-penetrating peptides (CPPs): Transportan, penetratin, pVEC and MAP (the training set); and averaged over the total number of amino acids in the sequence. Here, the Z₃ value, mainly describing polarity, had the highest predicting power. A number of protein and random sequences where searched, for sequences falling within the bulk property value Z_{Σ} interval obtained from the training set.

The descriptor interval used where: Z_{Σ1}<0.2 and Z_{Σ2}<1.1 and Z_{Σ3}<−0.49 and Z_{Σ4}<0.33 and Z_{Σ5}<0.95 and Z_{Σ5}>0.12.

### Results

Searching either a random- or natural protein sequence, sequences corresponding to CPPs appear clustered in blocks. This behaviour is due to the existence of “transport motors”, i.e. shorter sequences with CPP characteristics, in the search window. For the GLP-1 and Angiotensin receptors, sequences corresponding to CPPs were correctly predicted by the above outlined method. Searching a random sequence of 10,000 amino acid length returns on average 32 block hits for a sliding window length of 18 amino acids. However, the number of hits is dependent of window length. Other CPPs where used as controls. The criteria outlined above holds true for all of them, with the possible exception of the Tat/poly Arg family of CPPs.

TABLE 7
Example of block phenomena in the search of CPPs in AT1
receptor with an sliding window of 18 aa. The transport motor
is the motif included in all sequences.

| Position | Sequence | Block start |
|---|---|---|
| 298 | NPLFYGFLGKKFKKYFLQ | Transport motor |
| 299 | PLFYGFLGKKFKKYFLQL | |
| 300 | LFYGFLGKKFKKYFLQLL | |
| 301 | FYGFLGKKFKKYFLQLLK | |
| 302 | YGFLGKKFKKYFLQLLKY | |
| 303 | GFLGKKFKKYFLQLLKYI | |
| 304 | FLGKKFKKYFLQLLKYIP | M511 |
| 305 | LGKKFKKYFLQLLKYIPP | |
| 306 | GKKFKKYFLQLLKYIPPK | |
| 307 | KKFKKYFLQLLKYIPPKA | |
| 308 | KFKKYFLQLLKYIPPKAK | |
| 309 | FKKYFLQLLKYIPPKAKS | |
| | | Block end |

### Example 2

### Peptide Synthesis (Describing Default Method of the Experiments below if not Indicated Otherwise)

Peptides were synthesized in a stepwise manner in a 0.1 mmol scale on a peptide synthesizer (Applied Biosystems model 431A, USA) using t-Boc strategy of solid-phase peptide synthesis. tert-Butyloxycarbonyl amino acids (Bachem, Bubendorf, Switzerland) were coupled as hydroxybenzotriazole (HOBt) esters to a p-methylbenzylhydrylamine (MBHA) resin (Bachem, Bubendorf, Switzerland) to obtain C-terminally amidated peptide. Biotin was coupled manually to the N-terminus by adding a threefold excess of HOBt and o-benzotriazole-1-yl-N,N,N′,N′-tetramethyluronium tetrafluorborate (TBTU) activated biotin (Chemicon, Stockholm, Sweden) In DMF to the peptidyl-resin. The peptide was finally cleaved from the resin with liquid HF at 0° C. for 30 min in the presence of p-cresol. The purity of the peptide was >98% as demonstrated by HPLC on an analytical Nucleosil 120-3 C-18 RP-HPLC column (0.4×10 cm) and the correct molecular mass was obtained by using a plasma desorption mass spectrometer (Bioion 20, Applied Biosystems, USA) or MALDI-TOF (Vaager STR-E, Applied Biosystems, USA), as described in (Langel, U., Land, T. & Bartfai, T. Design of chimeric peptide ligands to galanin receptors and substance P receptors. Int J Pept Protein Res 39, 516-22. (1992)).

Cell Culture (Describing Default Method of the Experiments below if not Indicated Otherwise)

Murine fibroblasts C3H 10T1/2, mouse neuroblastoma N2A cells and COS-7 cells were grown in 10 cm petri dishes in Dulbecco's Modified Eagle's Media (DMEM) supplemented with 10% fetal calf serum (FCS), 2 mM L-Glutamine, 100 U/ml penicillin and 0.1 mg/ml streptomycin at 37° C. in a 5% CO₂ atmosphere. The cells were seeded and replated every fifth day. COS-7 and 10T1/2 cells were trypsinized when seeded while the N2A cells were suspended by mechanical force by adding media to the cells. Before starting the experiments, the cells were grown to confluency and then seeded and diluted two times in media before adding to 24-well plates (approximately 60 000 cells/well).

High throughput Screening (HTS) of Cargo Delivery Efficiency.

By using CPP—S—S-cargo constructs where the cargo is labelled with the 2-aminobenzoic acid fluorophore and the CPP with the 2-nitrotyrosine quencher, it is possible to monitor, in real time, the intracellular degradation of the disulfide bond resulting from the reducing intracellular milieu, and hence the cellular uptake of the constructs by in increase in apparent fluorescence.

### Peptide Uptake and Outflow Studies in Cells in Suspension or Attached

Cells were detached with trypsin (Invitrogen, Sweden), dissolved in culture media and centrifuged (1000×g for 10 min at RT). The cells were resuspended, counted and aliqoted in HKR on Ice, 300 000 cells/tube. Abz-labelled peptide was incubated for 15 and 30 min together with the cells in suspension, on a shaking 37° C. water bath. To stop the uptake or outflow, trypsin solution was added for 3 min. The cells were spun down at 1000×g for 10 min at 4° C. The pellets were resuspended in HKR for fluorescence detection, or for the outflow samples, incubated again with peptide-free HKR. Fluorescence was read at 320/420 nm on a Spectramax Gemini XS (Molecular Devices, Calif.). The intracellular concentrations were calculated from a standard curve of Abz-labelled peptides. The average cell volume of Caco-2 cells was determined by using a Coulter 256 channelizer (Coulter Electronics Ltd. CA). Additionally, a varianty of the same assay was applied: the cells were seeded in a 24well plate at a density of 100 000 cells/well, the day before. Cells are incubated at 5 μM peptide concentration for 30 min at 37° C. Trypsin treated, washed and lysed in hydrochloric acid. The fluorescence was measured as described for the suspension assay.

### Materials and Methods

### Cells

Human melanoma cells Bowes, were cultured in MEM using standard cell culturing techniques, and seeded at density of 100 000 cells/well in 24 well-plates the day before experiments were conducted.

### Results and Discussion

TABLE 20
Examples demonstrating the quantitative measurements for the ability of
various CPPs to enter Bowes cells when added for 30 min at
37° C. at 5 μM concentration.

| Fluoresceinyl peptide | % of added fluorescence in cell lysate |
|---|---|
| Transportan | 10.5 |
| pVEC | 4.6 |
| YTA-2ps | 3.5 |
| LRSW-3 | 1.9 |
| TP10 | 1.8 |
| LRSW-1 | 1.4 |

Table 20 Shows data from the preferred method to verify that the peptide is in fact a cell-penetrating peptide: Peptide uptake and outflow studies in cells in suspension or attached (see below). Approximately 50 peptides have been screened so far. For negative control the non-membrane permeable sugar polymer fluoresceinyl Dextran is added to the cells in the same manner, the percentage found in cell lysate of Dextran is always<0.5%. Note that YTA-2 ps, LRSW-1 and LRSW-3 are examples of de novo designed artificial peptides.

### Example 3

### Sequence Prediction

Several randomly generated sequences of 10.000 amino acids length were searched. The amount of hits varied with window length, but held at around 3% of the total sequences. Around 500 G protein coupled receptor sequences were searched, about 0.015% of the possible sequences was found to mach the CPP criteria, perhaps demonstrating that CPPs are selected against in nature.

The criteria outlined, successfully predicted around 95% of published CPP sequences.

### Example 4

### Cell-Selective CPPs

### Tissue Specificity of CPPS:

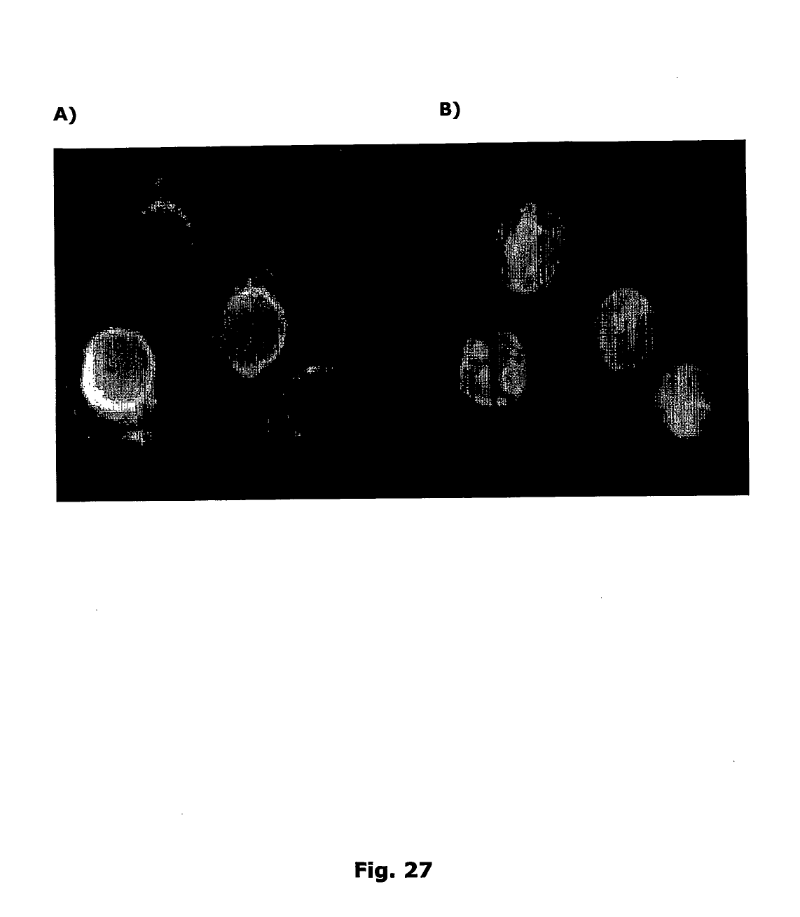

As the cellular uptake of CPPs is likely to depend on membrane properties and membrane potential of the target cell, it is possible to obtain cell specific CPPs. Using the criteria outlined above, a biased library of peptides will be generated. The library is then be tested for uptake/cargo delivery efficiency by e.g. using the method described above. Briefly, by using CPP—S—S-cargo constructs, wherein the cargo is labeled with the 2-aminobenzoic acid fluorophore and the CPP with the 3-nitrotyrosine quencher, it is possible to monitor (in real time) the intracellular degradation of the disulfide bond. Hence the cellular uptake of the constructs and the cargo delivery efficiency of the CPP can be measured by the increase in apparent fluorescence (See FIG. 27). This method eliminates the experimentally difficult step of distinguishing between internalised and outer membrane bound peptide.

### Method of Determining the Specific Cleavage of YTA-2 ps of the Matrix Metallo Proteinase-2 (MMP-2):

As Illustrated above and In FIG. 27, the inventors have been able to show that the CPP-part of a selective CPP (YTA-2) can efficiently enter cells both at 37° and 4° C. (data not shown). In addition, the “Inactivator” made the peptide less active in translocation over the cell membrane. The next step in the development of this technique is to check the specific cleavage of YTA-2 ps by active MMP-2. It is performed by a fluorescence/quencher assay, wherein the MMP-2 substrate YTA-2 ps upon cleavage increases in fluorescence intensity. The correct cleavage is further checked by mass spectrometry.

### Example 5

Design of a functional protein mimicking CPP, examplified by a new effector-mimic-CPP for treatment of Insulin deficiency in non-insulin dependent diabetes mellitus:

### Background

GPCR-ligand interactions and their mimicry in disorders:

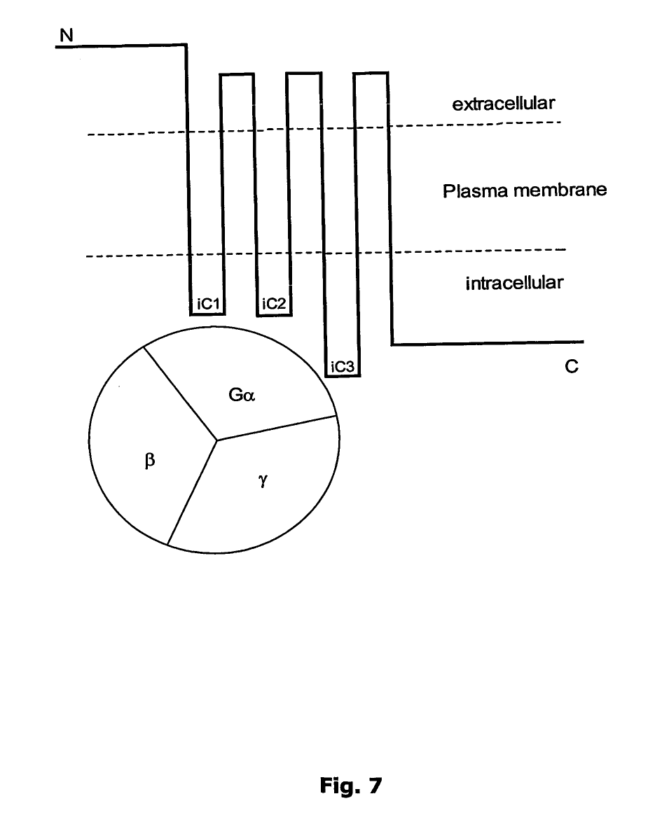

The interactions between 7™ spanning receptors with their respective G-proteins are well defined and specific. In fact, these interactions are the analogues of DNA/DNA interactions between specific and well-defined proteins. 7™ receptors are G-protein coupled receptors (GPCR) and as such, they expose amphipathic α-helical motifs, which are suggested to be responsible for G-protein binding. It has been demonstrated that parts of the GPCR's third intracellular loop, but sometimes also other loops are involved in signaling. On the appended schematic drawing FIG. 7, such a 7™ receptor-G-protein complex is presented. Synthetic peptides from the intracellular parts of GPCRs can mimic the interaction of the GPCR and G-protein, i.e. conveying an activated receptor signal, as has been demonstrated in cellular fragment systems.

In the present example, the inventors have demonstrated that a novel CPP, derived from one of said intracellular parts of a GPCR, can both act as a cell-penetrating peptide, as well as at the same time mimic the function of an activated receptor in the cells, e.g. mimic the interaction of the GPCR and G-protein in the target cell.

Non-Insulin Dependent Diabetes Mellitus, NIDDM, and Glucagon like Peptide 1 Receptor, GLP-1R

Non-insulin dependent diabetes mellitus, NIDDM, also known as type 2 diabetes mellitus, T2DM, is characterised by complex hormonal disturbances and insulin resistance.

Treatment of NIDDM is complicated due to the complexity of the disorder, as well as to poor understanding of the mechanisms behind it. Probably, several key cellular and molecular mechanisms in NIDDM still remain to be defined. Despite the lack of comprehensive knowledge of mechanisms of NIDDM, recent achievements in diabetes research have revealed some promising targets for studies and treatment.

Non-insulin dependent diabetes mellitus, NIDDM, is currently treated by hormone replacement with insulin, with insulin releasers, or insulin sensitizers. However, none of these treatments is fully satisfactory in controlling serum glucose levels. Glucose dependent insulin release is partly controlled by the activation of the G-protein coupled receptor glucagon like peptide 1 receptor, GLP-1R, rendering it a promising target for a new NIDDM therapeutic agent. The existence of an Ideal endogenous agonist for GLP-1R, the glucagon like peptide 1, GLP-1, has been known for almost 15 years. However, its pharmacological exploitation has so far failed due to short half-life of the peptide when administered i.v. and due to loss of agonist efficacy of most of the synthetic analogues.

The inventors herein show that GLP-1R agonist-mimics, based on intracellular loop 3 (iC3) peptides of GLP-1R receptors can mimic the active state of the agonist occupied receptor in signalling to initiate Insulin release.

The design and synthesis of peptides derived from GLP-1R iC3 are demonstrated, which induce insulin release in rat pancreatic islets. These approaches greatly facilitate the study of the mechanisms underlying the activation of insulin-release by GLP-1, and provide physical libraries of substances, which will stimulate insulin release, and thus serve as potent leads for the development of drugs for NIDDM treatment.

### GLP-LR Fragments as GLP-1 Receptor Agonist Mimics.

Synthetic peptides derived from the third intracellular loop, IC3, of GLP-1 receptor activate GTPase and adenylate cyclase (AC) with EC50=100 nM. The iC3 peptides are powerful enzyme activators, often a 6- to 13-fold activation of the AC is achieved. Additionally, these peptides may serve as tools for study of promiscuity of the GLP-1 receptor in signal transduction to G-proteins.

The short, 12-20 amino acids long iC3 peptides mimic the interactions of the agonist occupied 7™ receptor proteins in vitro. The peptides can be furthermore be connected to cellular transporters, such as Transportan, for more efficient penetration into the cell interior where these interactions take place in in vivo studies.

### Summary of Results

The inventors have designed, synthesized and tested for insulin release a novel octapeptide derived from glucagon-like peptide-1 receptor, GLP-1R with sequence IVIAKLKA-amide (GOP). This peptide mimics the action of the parent protein, GLP-1R, a seven transmembrane spanning protein known to initiate insulin release in pancreatic islets followed by recognition of the GLP-1 peptide hormone. Analogues of this peptide are novel cell-penetrating peptides, CPPs, that are able to translocate cell membranes in the tested cells. These data demonstrate a novel possibility to design agonists of a desired protein, which are cell-penetrating by themselves.

### Methods

### Cellular Penetration of Biotin-Labelled Peptide

The medium containing serum was exchanged for a serum-free medium and water solution of the peptide was added directly into the medium to reach the concentration of 10 μM. A negative control was always used, because living cells all the time have some biotin inside. To control cells pure water instead peptide solution were added, and further handled alike all other. The cells were incubated for 1 h at 37° C. or 4° C. in 5% CO₂ enriched air. The cells were washed three times with PBS, fixed and permeabilised with methanol for 10 min at −20° C., washed again with PBS and Incubated for 1 h in a 5% (w/v) solution of fat-free milk in PBS in order to decrease non-specific binding. The peptides were visualised by staining with 0.1 μM streptavidin-FITC in the same solution for 1 hour at room temperature. The cell nuclei were visualised by staining with DNA with Hoechst 33258 (0.5 μg/ml) for 5 min, thereafter the coverslips were washed 3 times with PBS and mounted in 20% glycerol in PBS. The images were obtained by Zeiss Axioplan 2 microscope (Carl Zeiss Inc., Germany).

### Insulin Release

Effects of the peptides on insulin secretion were assessed in pancreatic islets from male Wistar rats weighing 200-250 g. Islets were isolated aseptically by colagenase digestion, and then cultured overnight at 37° C. in RPMI 1640 culture medium supplemented with 10% heat-inactivated fetal calf serum. After culturing, analysis of insulin secretion was performed by incubation at 37° C. for 1 h in batches of 3 islets, each in 300 μl of Krebs-Ringer bicarbonate buffer with 10 mM Hepes and 2 mg/ml bovine serum albumin, pH 7.4. The incubation medium contained either 3.3 or 16.7 mM glucose, with or without peptide. In one experimental series, batches of 5×10⁵ Rin m5F cells were incubated under similar conditions, except that glucose was omitted from the medium. Aliquots of the incubation medium were taken for radioimmunoassay of Insulin. Insulin secretion is expressed as IU insulin/islet/h and IU insulin/5×10⁵ cells/h, respectively. Statistical significance was evaluated with the Student's t-test with p<0.05 regarded as significant

### Results and Conclusions

A short peptide derived from the GLP-1R-sequences is able to dose-dependently and glucose dependendently increase insulin secretion form isolated rat pancreatic islets. Furthermore the peptide is able to increase insulin secretion and decrease blood-glucose levels when injected i.v. in rats.

### Cell Penetration of GLP-1R Loop Derived Peptides

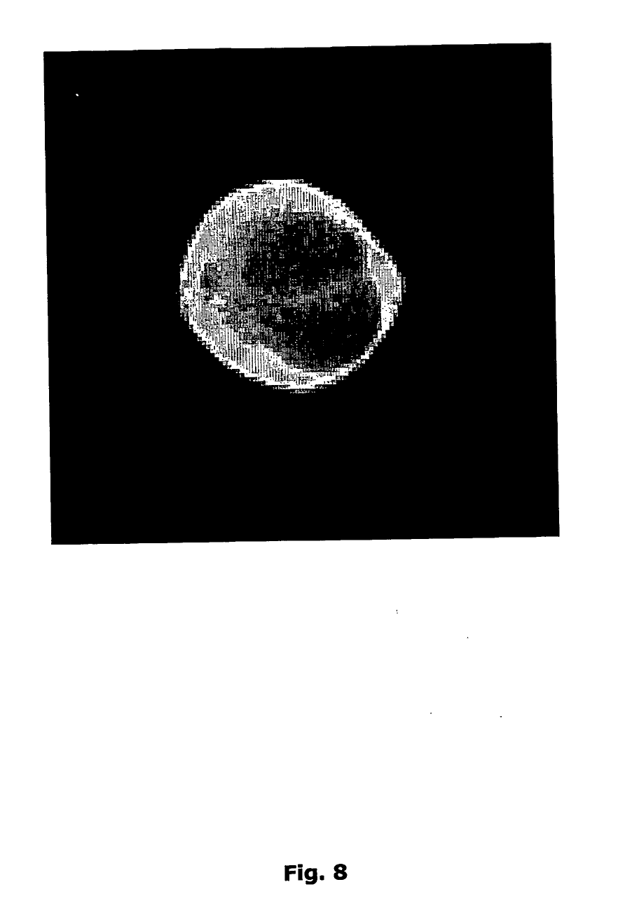

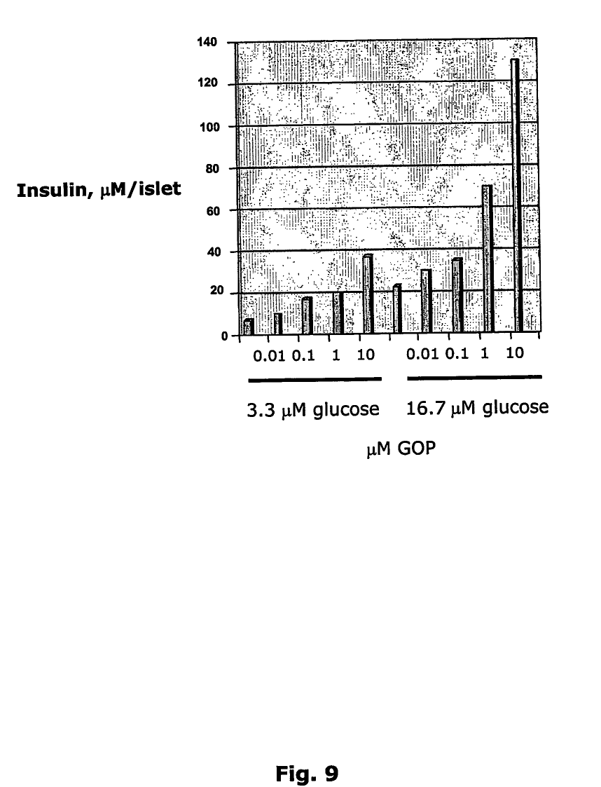

In FIG. 8, the cellular penetration of the GOP analogue M569 is illustrated. The inventors have been able to further develop the GLP-1R derived peptides described above. A new generation of these peptides, which includes the sequence IVIAKLKA is characterized to have cell-penetrating properties, activate G-proteins and increase insulin release when incubated with rat pancreatic islets. The GLP-1R derived peptide M569 shows temperature independent penetration into the human Bowes melanoma cells and the same intracellular localization of the peptide is registered at 4° C. as well (not shown).

### Insulin Release

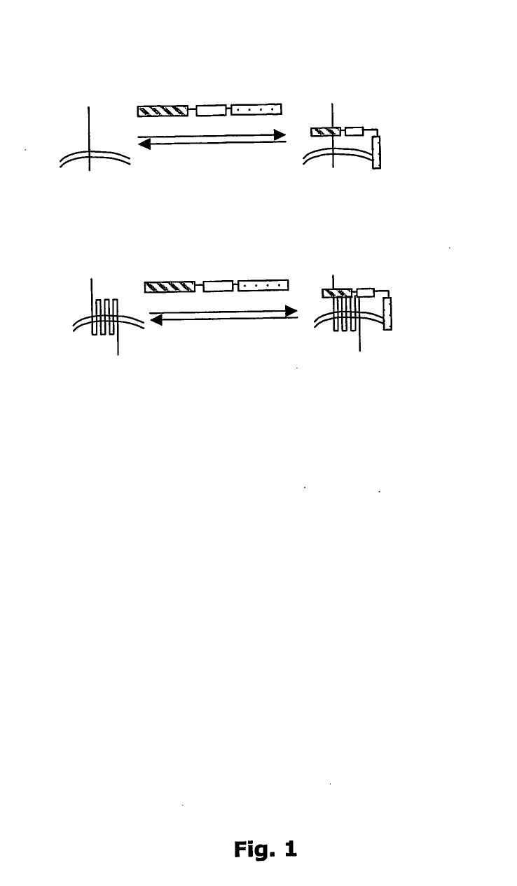

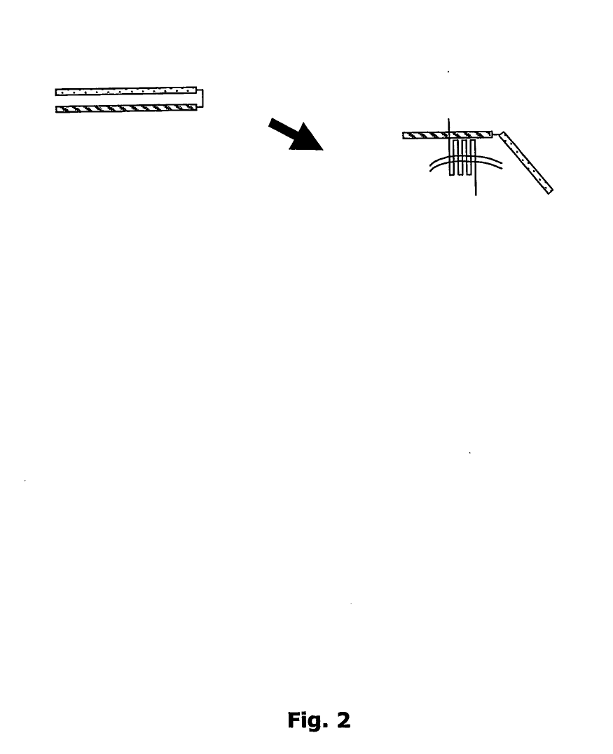

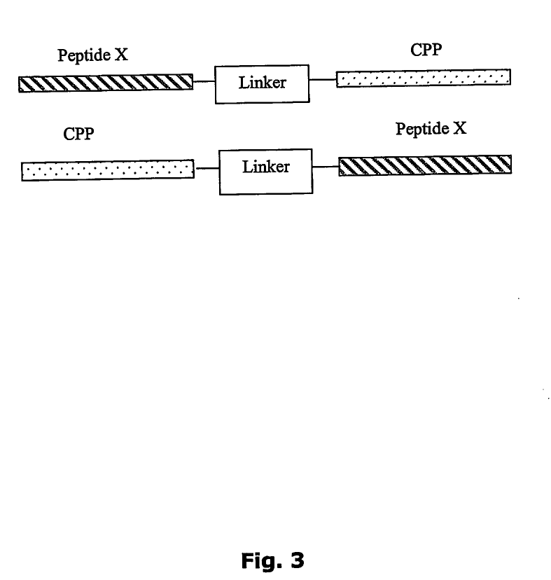

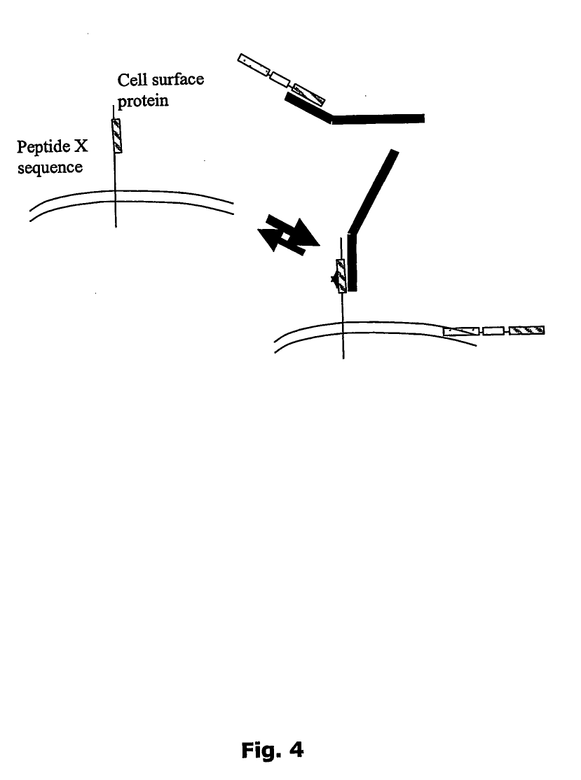

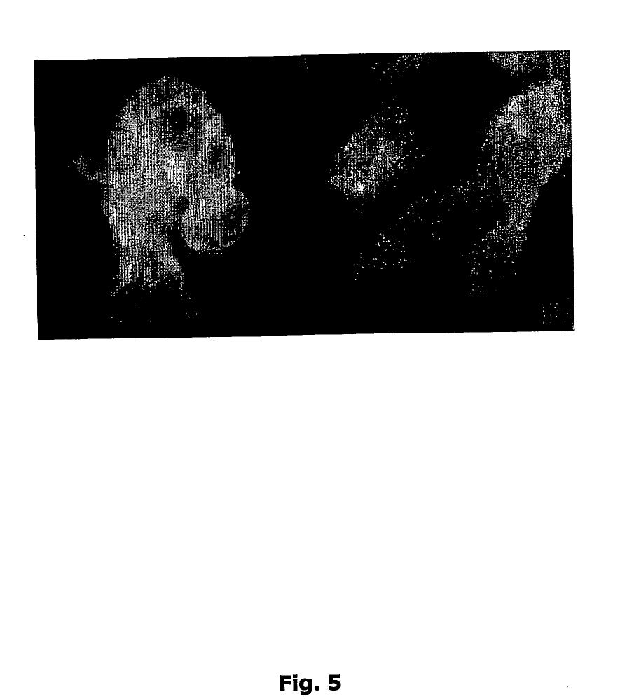

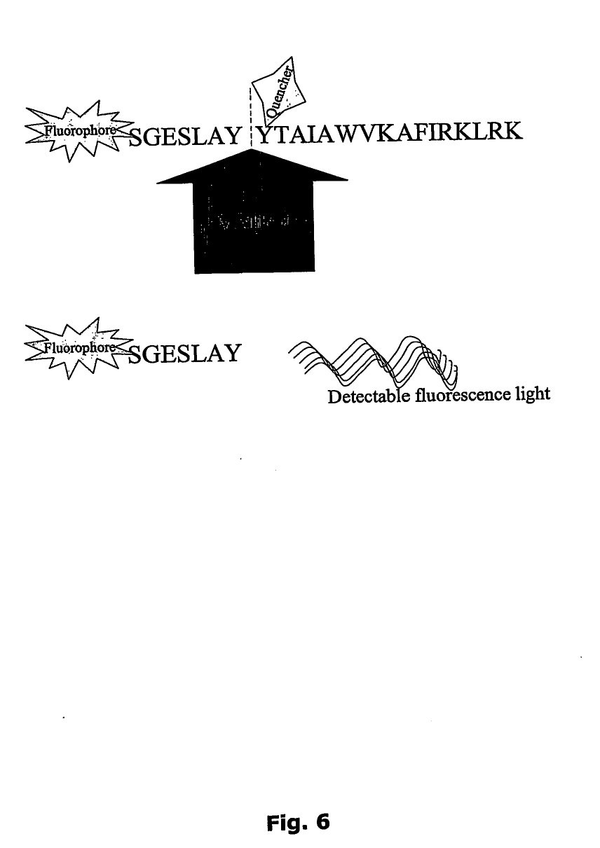

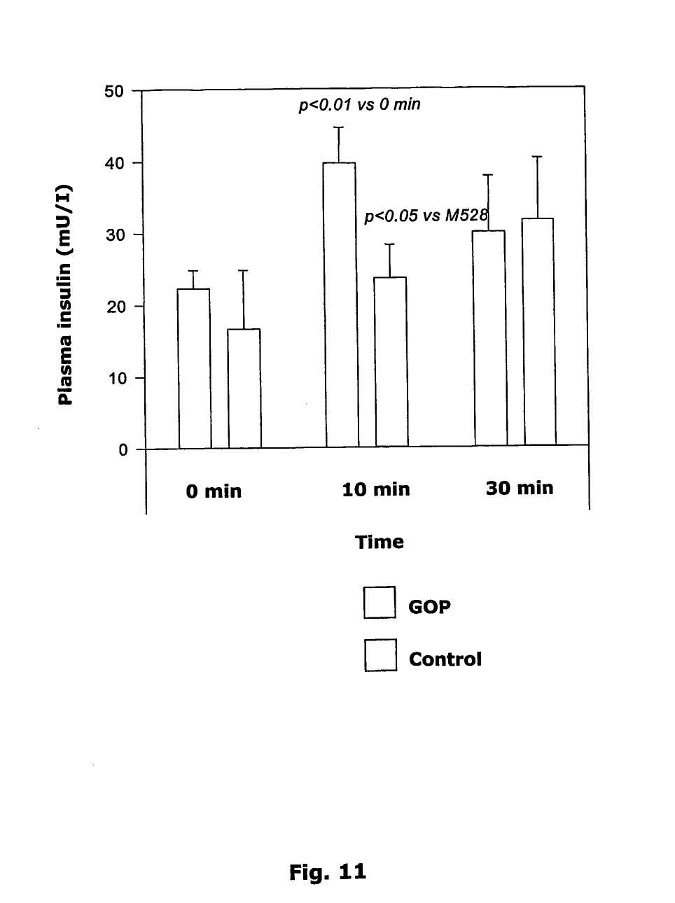

In the absence of the peptide GOP, 16.7 mM glucose stimulated Insulin release almost 3-fold as compared to basal release at 3.3 mM glucose (FIGS. 1 and 11). GOP (10 μM) stimulated insulin release 5-fold (p<0.001) at 3.3 mM glucose, and at 16.7 mM glucose, the stimulation by 1 and 10 FM GOP is 4-fold.

In vivo Effect of GOP (M528) on Insulin Release and Blood Glucose Level

Healthy male Wistar rats, weighing approximately 250 g, were fasted over-night. After an initial blood sample obtained by incision of the distal tail vein (0 min), GOP (M528, 100 nmol/kg; n=4) was injected intraperitoneally, followed by another i.p. injection of glucose (1 g/kg). Control rats (n=4) were injected with saline and glucose. Additional blood samples were taken from the tail vein after 10 and 30 min. Blood glucose levels were determined by a glucose oxidase method and plasma insulin levels by radioimmunoassay.

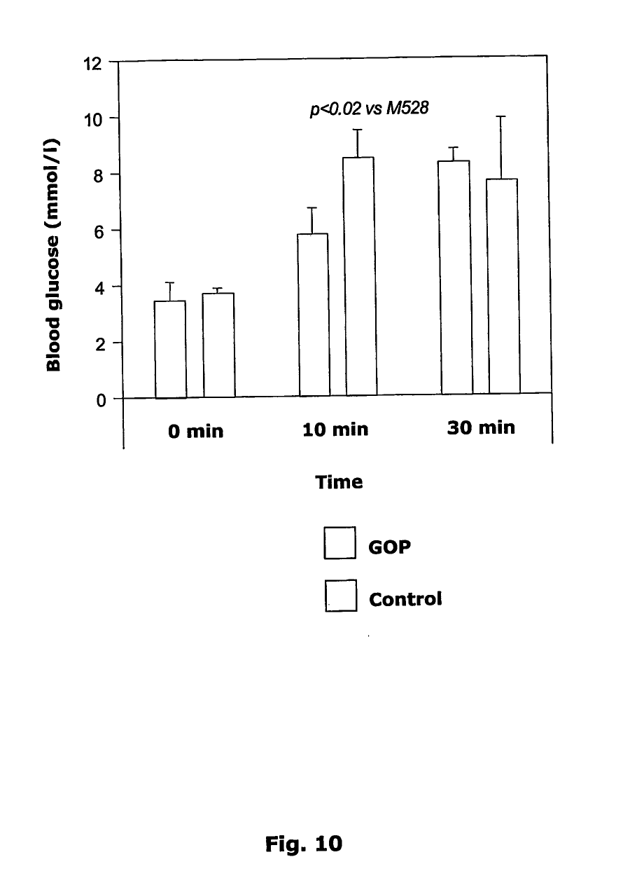

As evident from FIG. 10 and FIG. 11, plasma insulin and blood glucose concentrations were similar in all rats before injections. After 10 min, plasma Insulin had increased significantly in the GOP-injected rats, compared to control rats (p<0.05) and to same group of rats at 0 min (p<0.01) (FIG. 10). In parallel, blood glucose levels were significantly lower in the GOP-injected rats relative to control rats at 10 min (p<0.02) (FIG. 11). After 30 min there were no differences between the two groups of rats regarding Insulin and glucose levels. The present data indicate a marked, however transient, effect of the peptide on insulin release in vivo. In conclusion, the GLP-1R derived peptides of the GOP family penetrate cell membranes, interact specifically with respective G-protein and Increase insulin release from rat pancreatic islets.

TABLE 8
GOP-peptide derivatives

| | | | Insulin release | | Insulin release | |
|---|---|---|---|---|---|---|
| | | | at 3.3 mM | | at 6.7 mM | |
| | | | glucose | | 1 glucose | |
| Peptide | sequence | MW | 10 μM | 100 μM | 10 μM | 100 μM |
| GOP | IVIAKLKA amide | 853.62 | | no | stim+ | stim++ |
| (GOP1) | | | | | | |
| GOP2 | (CIVIAKLKA)₂ amide | 1928.47 | | | | |
| GOP3 | IVIAKLRA amide | 881.63 | | stim++ | stim+ | stim++ |
| GOP4 | IAIAKLKA amide | 825.59 | | inh− | inh−− | inh− |
| GOP5 | IVIAKLAA amide | 796.56 | | no | inh− | no |
| GOP6 | all-D-(IVIAKLKA) amide | 853.62 | | stim+ | inh− | no |
| GOP7 | I-(N-Me-V)-IAKLKA amide | 866 | | | | |
| GOP8 | I(all-N-Me(VIAKLKA)) | 855.63 | | | | |
| | amide | | | | | |
| GOP9 | IV-Oca-KA amide | | | | | |
| GOP10 | AKLKAIVI amide | 853.62 | | | | |
| GOP11 | IAIAKLAA amide | 768.53 | | inh− | inh− | inh−− |
| GOP12 | VIAKLK amide | 669.5 | | | | |
| GOP13 | IVI-(N-Me-A)KLKA amide | | | | | |
| GOP14 | IVIAKLK-(N-Me-A) amide | | | | | |
| GOP15 | IVI-aib-KLKA amide | | | | | |
| GOP16 | IVVSKLKA amide | 855.6 | | no | no | no |
| GOP17 | IVIAKLKA-COOH | | | | | |
| GOP18 | I-norV-IAK-norL-Cit-A- | | | | | |
| | COOH | | | | | |
| GOP25 | I-OmTyr-IAKLKA amide | 931.67 | | | | |
| GOP26 | IVIA-Cit-LKA amide | 882.71 | | | | |
| GOP52 | VVKK amide | 471.37 | | | | |

Series of GOP Analogues will be Chosen Among the Following:

IVV-X-KLKA, IVI-X-KLKA, IV-X₁-X-KLKA

Where X₁ is an amino acid and X is a linker.

### Example 6

### Design of a Non-Receptor Functional Protein Mimicking CPP, Exemplified by a new Effector-Mimic-CPP Corresponding to the N-Terminal Sequences of the Prion Protein (PrpC)

### Background

The N-terminal sequences of the prion protein (PrPC) are very similar to constructed signal peptide-NLS chimera, shown to function as cell-penetrating peptides (CPPs) (Vidal, P. et al. Interactions of primary amphipathic vector peptides with membranes. Conformational consequences and Influence on cellular localization. J Membr Blol 162, 259-64. (1998)). Based on these sequence similarities, the inventors tested the hypothesis that also the PrPC derived sequence from mouse with non-cleaved signal sequence is active as a CPP. The inventors found that mouse PrPC(1-28) is indeed a CPP, with the ability to carry a cargo avidin into cells, based on a standard fluorescence assay technique. In distilled water, the PrPC(1-28) peptide is strongly aggregated above 1 mM concentration, and has a dominating 0 structure. The findings have significant implications for the understanding of how prion proteins with intact N termini may invade cells and of the secondary structure conversion to p structure that is associated with the conversion to the scrapie form of the protein.

The following peptide sequences were compared:

The chimeric CPP, i.e. the hydrophobic sequence from gp41 of HIV (1-17)+NLS from SV40 large antigen T (18-24): MGLGL HLLVL AAALQ GAKKK RKVC (1)

Mouse PrpC(1-28): MANLG YWLLA LFVTM WTDVG LCKKR PKP (2)

Human PrpC(1-28): MANLG CWMLV LFVAT WSDLG LCKKR PKP (3)

Bovine PrPC (1-30): MVKSK IGSWI LVLFV AMWSD VGLCK KRPKP (4).

The general features of both types of sequences is a mainly hydrophobic signal peptide part of 16-23 residues followed by a NLS part of about 7 residues, with high positive charge.

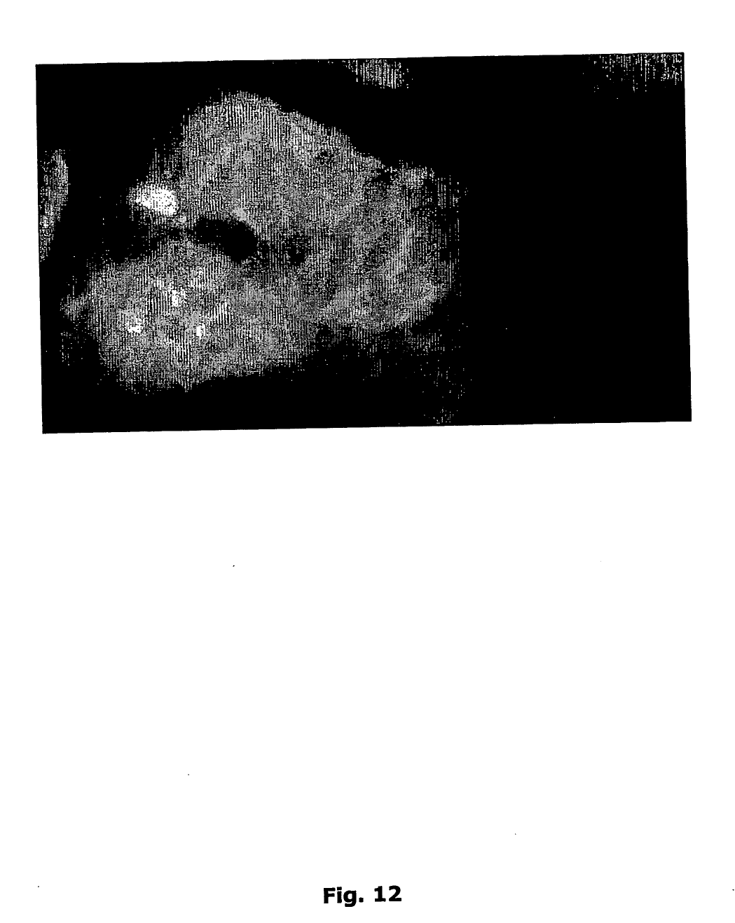

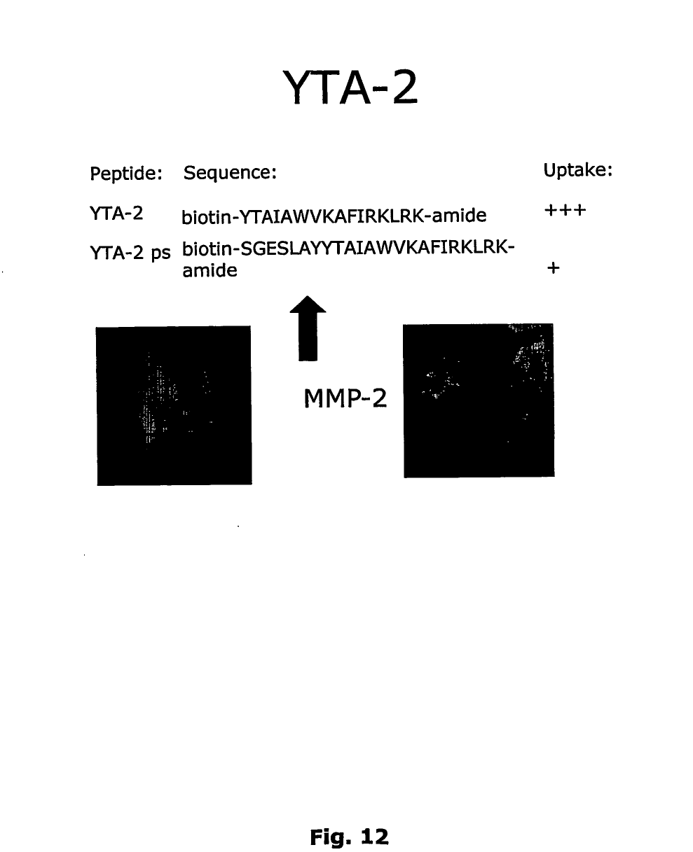

Mouse PrpC(1-28) was synthezised with a blotin lable in the N terminus to investigate its cell-penetrating properties. The internalisation of the peptide was monitored through the coupled flourescine, as previously described in example 7 and conducted in cultured N2A cells. The cell penetration properties were investigated for the peptide itself and for the peptide carrying a large cargo of the avidin protein (65 kD). The protocol followed was the same as used in previous experiments (Kilk, K. et al. Cellular Internalization of a cargo complex with a novel peptide derived from the third helix of the Islet-1 homeodomain. Comparison with the penetratin peptide. Bioconjug Chem 12, 911-6. (2001), Magzoub, M., Kilk, K., Eriksson, L. E., Langel, U. & Graslund, A. Interaction and structure induction of cell-penetrating peptides in the presence of phospholipid vesicles. Biochim Biophys Acta 1512, 77-89. (2001)) demonstrating CPP properties of the pIsl peptide sequence derived from the homeodomain of the rat transcription factor Islet-1 (Inoue, A., Takahashi, M., Hatta, K., Hotta, Y. & Okamoto, H. Developmental regulation of islet-1 mRNA expression during neuronal differentiation in embryonic zebrafish. Dev Dyn 199, 1-11. (1994)). FIG. 12 shows fluorescence microscope pictures clearly indicating the CPP efficiency and perinuclear localization of both preparations, PrpC(1-28) without and with the avidin cargo.

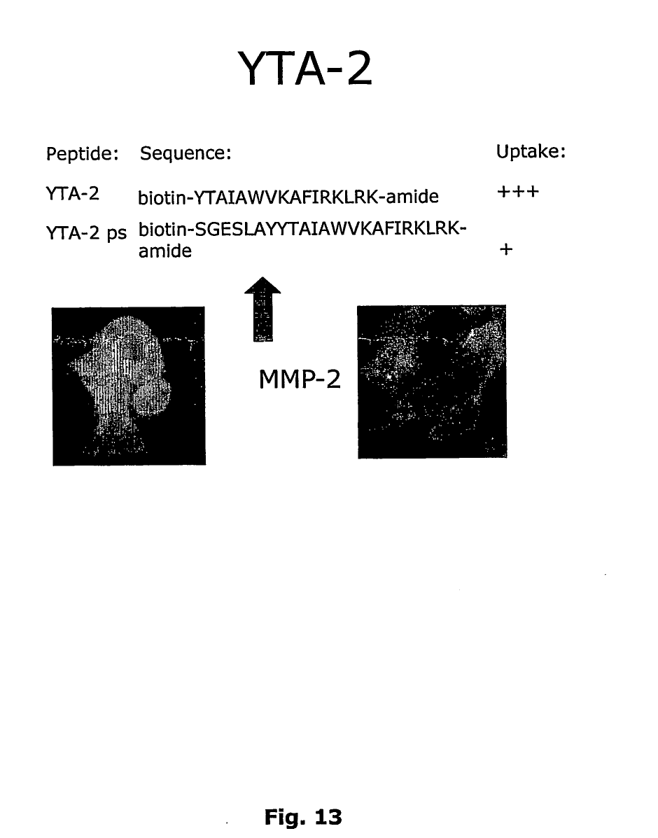

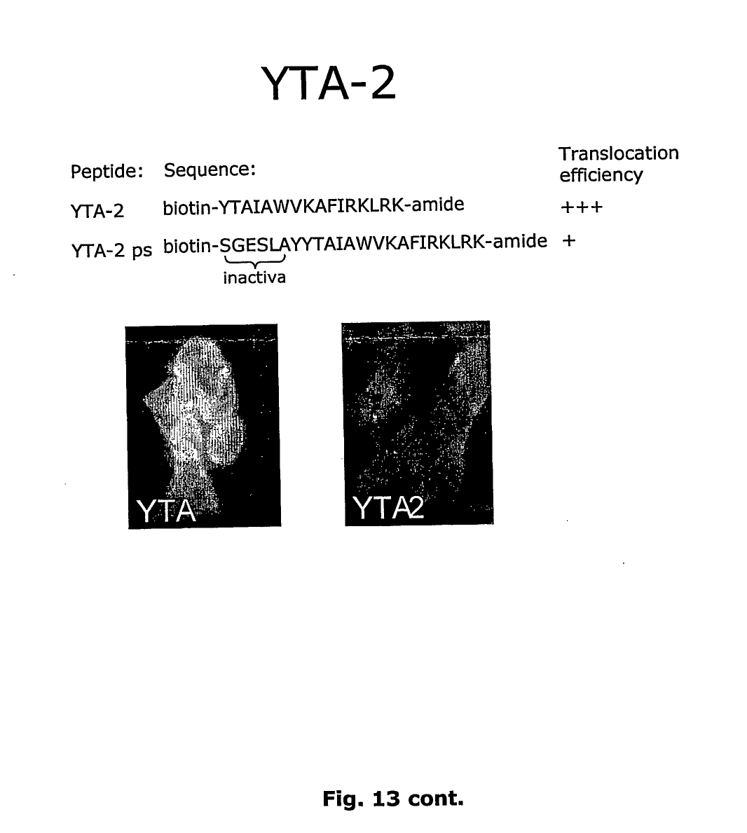

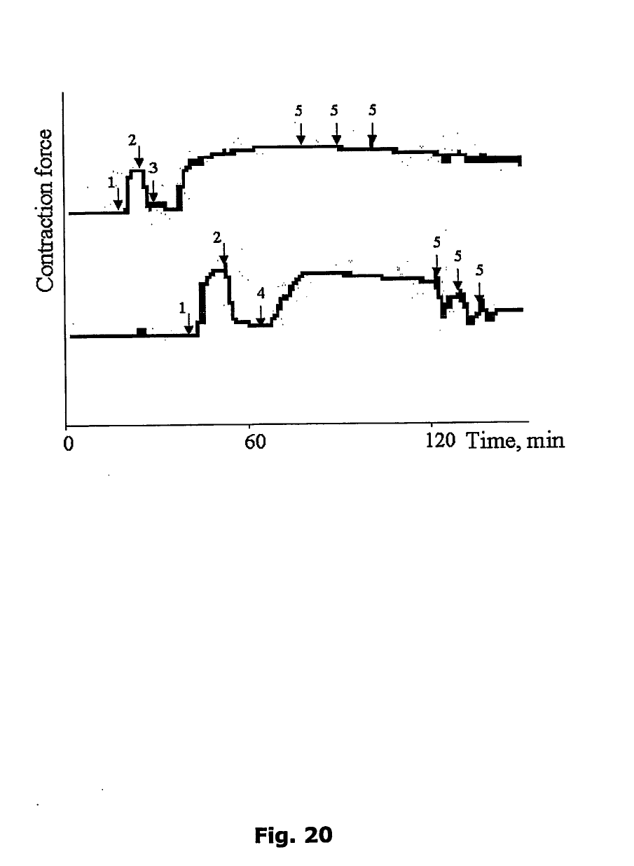

The secondary structures of the peptide were further investigated, now without biotin attachment, in aqueous solution and In various membrane mimetic solvents by CD and NMR spectroscopy. FIG. 20B shows a CD spectrum of 1 mM peptide in distilled water, with features typical of a significant 0 structure contribution. Addition of salt increased the β structure contribution (data not shown). Parallel ¹H NMR experiments yielded no evidence of a resolved spectrum and attempts to investigate diffusion showed that peptide aggregates had formed in the sample that were larger than could be measured by the NMR technique. In the presence of negatively charged phospholipid vesicles the in structure contribution of the peptide was considerably increased (FIG. 13). FIG. 20C shows a partial NMR TOCSY spectrum of the peptide in SDS micelles, which dissolves the peptide aggregates so that a well resolved ¹H NMR could be obtained and the resonances assigned. The secondary chemical shifts of the Has along the peptide chain give clear evidence of induced α-helical structure. This chameleon-like behaviour of the peptide in various solvents essentially mirrors observations from other CPPs, like penetratin (Derossi, D., Joliot, A. H., Chassaing, G. & Prochiantz, A. The third helix of the Antennapedia homeodomain translocates through biological membranes. J. Biol. Chem. 269, 10444-10450 (1994)) and pIsl (Kilk, K. et al. Cellular internalization of a cargo complex with a novel peptide derived from the third helix of the islet-1 homeodomain. Comparison with the penetratin peptide. Bioconjug Chem 12, 911-6. (2001)) except that the aggregation and P structure contribution in aqueous solution is more pronounced and even more dependent on ionic strength and peptide concentration for mouse PrPC(1-28) than for penetratin and pIsI.

### Example 7

### Design of a Functional Protein Mimicking CPP, Examplified by New Effector-Mimic-CPPs Corresponding to Amyloid Precursor Protein (APP) and Presenilin-1 (PS-1)

### Background

Alzheimer's disease is the most common form of dementia in the elderly, and although the disease was discovered almost a century ago, no cure nor exact mechanism of action have been discovered. The most classic hallmark of the disease is protein aggregates, called senile plaques. These senile plaques consist mainly of a peptide derived from the amyloid precursor protein, called β-amyloid. This β-amyloid peptide is created during the processing of the amyloid precursor protein, where two proteins called presenilin-1 and presenlin-2 are thought to be involved.

### Summary

The example demonstrates that the amyloid precursor protein (APP) and presenilin-1 (PS-1) have specific peptide sequences, so called cell-penetrating sequences, which can help them to translocate across cell membranes and be internalised by living cells. This might be used as a mechanism by which secretory APP, or any other neuroprotective peptide, can be internalised and thereby exert its neuroprotective properties.

### Materials and Methods

### Cellular Assays

### Cell Culture:

Mouse neuroblastoma cells, N2A, were cultivated in Dulbecco's minimal essential medium with Glutamax-I, supplemented with 10% (v/v) fetal bovine serum, penicillin (100 U/ml) and streptomycin (100 μg/ml).

### Cellular Internalisation Assay:

The cells used for Internalisation were seeded out on round glass coverslips in 24well plates. One day post seeding, the cells were semi confluent, and the medium was changed to serum-free medium. The fluorescein-labelled peptides were added, with a final concentration of 10 μM. After 60 min of incubation at 37°, the cells were washed 3 times with 1 ml of Hepes-Krebbs-Ringer-(HKR) buffer and fixed with 3% paraformaldehyde solution in phosphate-buffered saline solution (PBS) for 10 minutes. The cells were then washed 3 times with 1 ml of HKR-buffer, and the coverslips were mounted and sealed for microscopy studies.

### Results

### Peptide Synthesis and Purification

The following peptides were synthesised, coupled to fluorescein, purified on HPLC and analysed on mass spectrometer (as described in Langel, U., Land, T. & Bartfal, T. Design of chimeric peptide ligands to galanin receptors and substance P receptors. Int J Pept Protein Res 39, 516-22. (1992)).

TABLE 9
Sequence, calculated molecular masses and measured molecular masses
of synthesised APP and PS peptides labeled with fluorescein

| Peptide | Sequence |
|---|---|
| APP (521-537) | KKAAQIRSQVMTHLRVI |
| APP (712-726) | IATVIVITLVMLKKK |
| WT | |
| APP (712-726) | IATVIFITLVMLKKK |
| mutant V717F | |
| APP (725-740) | KKKQYTSIHHGVVEVD |
| PS-2 (86-110) | KVHIMLFVPVTLCMIVVVATIKSVR |
| PS-1 (151-162) | VVLYKYRCYKVI |

### Cellular Internalisation Assay

The cell type used for the internalisation assays with the fluorescein'tagged peptides was N2A mouse neuroblastoma cells. This cell-line is commonly used in association with Alzheimer's disease studies, and serves as a good model cell-line for these internalisation assays where peptides derived from proteins involved in Alzheimer's disease were examined. These Internalisation assays were performed at 37° C., therefore the internalisation by endocytosis cannot be excluded.

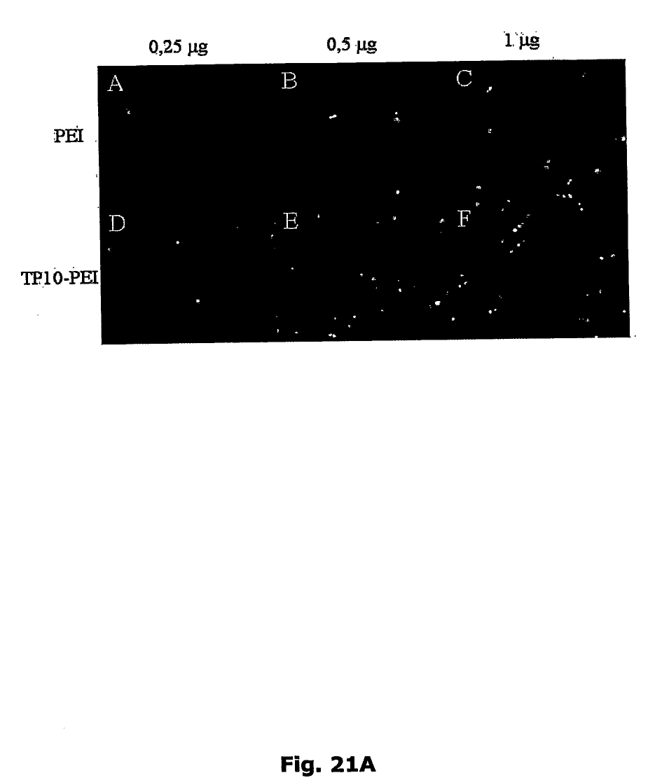

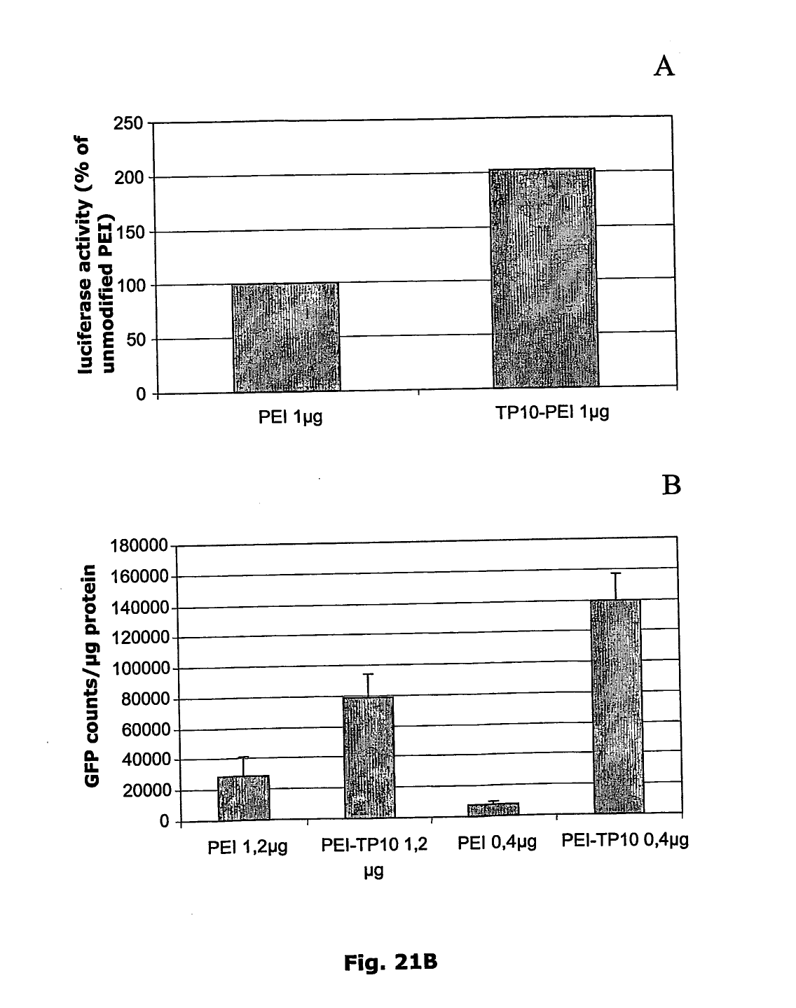

FIG. 21 shows cellular localisation of peptides in N2A cells at a concentration of 10 μM A) and B) Cells treated with APP 521-536 labeled with fluorescein. C) Cells treated with APP 725-740 labeled with fluorescein. D) Cells treated with PS-1 97-109 deletion analogue coupled to fluorescein. E) Cells treated with the known CPP penetratin labeled with fluorescein. F) Untreated cells.

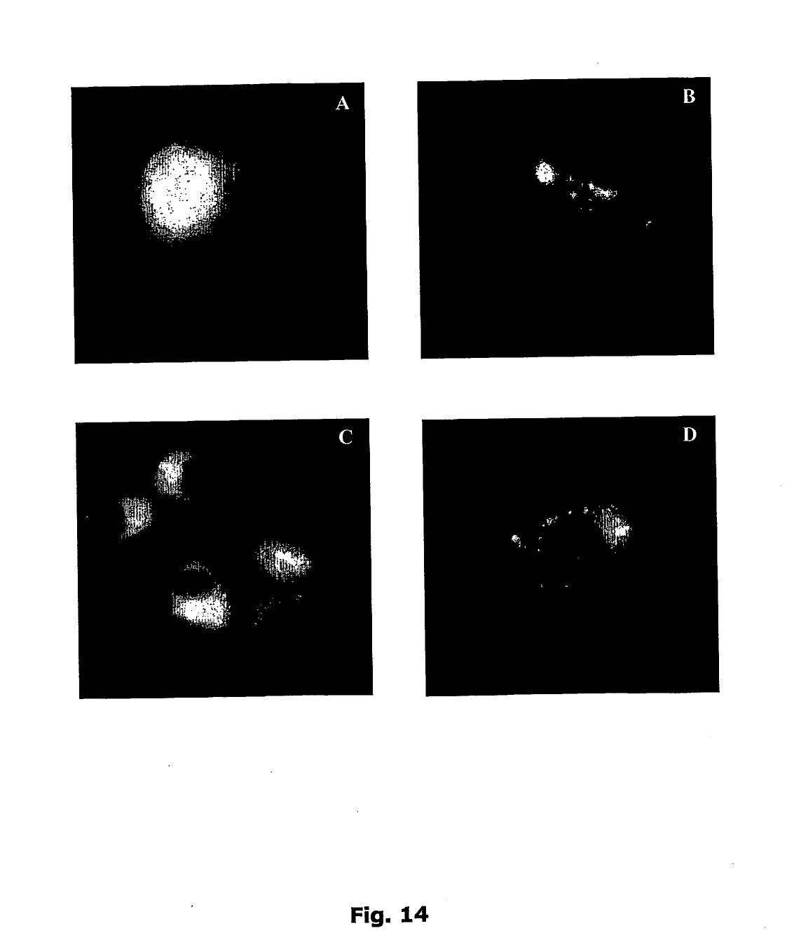

The internalisation experiments demonstrate that APP (521-536), which is derived from the extracellular part of the protein, has cell-penetrating abilities (FIGS. 14 A and B), localising both in the nucleus and the membrane. This is a putative pathway by which the secretory amyloid precursor protein (sAPP) is internalised. This fact is interesting since this fragment has been shown to protect neurons against hypoglycemic damage and glutamate neurotoxicity thus acting as neuroprotective agent. The presenilin-1 (97-109) deletion analogue, derived from a membrane spanning- and first extracellular loop part, also showed cell-penetrating abilities. However, this peptide is mostly localised in the cytosole, but can also be detected in the plasma-membrane and nucleus (FIG. 14D). APP (725-740) shows an increased fluorescence compared to the background, which seems to be situated mostly to the cytosol. However, this does not seem as clear as in the earlier mentioned peptides, and recent experiments have shown that it is not internalised at 4° C., which suggests that the slight increase in fluorescence that was observed is probably caused by endocytosis. Identical increase in fluorescence could also be observed for presenilin-1 (151-162) (not shown), which is a peptide derived from the first intracellular loop in presenilin-1. The internalisation experiments with APP (712-726) WT and APP (712-726) V717F mutant, do not reveal any no uptake. The reason behind this is probably that as soon as these peptides were added to the 24-well plate, they formed aggregates. This tendency to aggregate was also observed earlier, especially when the crude product was dissolved for purification on HPLC.

### Example 8

Design of a functional protein mimicking CPP, exemplified by a new effector-mimic-CPP corresponding to a synthetic peptide derived from the intracellular C-terminus of anglotensin 1A receptor.

Example 8 discloses a novel vasoconstrictor, more precisely to a synthetic peptide derived from the intracellular C-terminus of angiotensin 1A receptor. The peptide is a cell-penetrating peptide and promotes contraction of heart coronary blood vessels.

### Background

Angiotensin receptors are members of the 7-transmembrane G-protein coupled receptor family and are important components of the blood pressure and electrolyte balance maintaining system in mammals. They exist in two types: AT1 (consists of ATLA and AT1B subtypes) and AT2, among which AT1 seems to be responsible for the mediation of almost all known systemic effects of anglotensin II. AT1 receptors are involved in contraction of smooth muscles in different tissue, e.g. in blood vessels, uterus, bladder, and some endocrine glands, and are widely distributed in kidney, liver, and in CNS. Antagonists of AT1 receptor are potential antihypertensive drugs and some non-peptide antagonists, e.g. lorsatan, have been successfully introduced in clinical use. Selective agonists for AT1 receptor, however, are not available today. Agonists would be of interest as potential drugs useful in the situations where vasoconstriction is required, e.g. chronical hypotension or migraine.

### Methods and Materials

### Cell Culture

Bowes melanoma cells (American Type Culture Collection CRL-9607) were cultivated in Minimal Essential Medium (MEM, Life technologies, Stockholm, Sweden) with glutamax supplemented with 10% fetal bovine serum, 1% penicillin-streptomycin solution, 1% non-essential amino acids and 1% sodium pyruvate.

### Cellular Internalisation Assay

The cells used for internalisation experiments were seeded at a density of 10.000 cells/well on round glass coverslips in 24-well plates. After one day, when they had reached about 50% confluency, the medium was changed to serum-free and blotinylated peptides were added directly into the medium. After 60 min of incubation at either 37° C. or 4° C., the cells were washed three times with PBS and fixed with 3% (w/v) paraformaldehyde solution in PBS for 15 min. For indirect detection of the biotin labeled peptide, the fixed cells were permeabilized with 0.5% Triton X-100 in PBS and sites for non-specific binding were blocked with 5% BSA in PBS. The blotin moieties were visuaialized by incubation of the treated cells with streptavidin-TRITC (Molecular Probes, Netherlands, 1:200) for 1 h at room temperature. The cell nuclei were stained with Hoechst 33258 (0.5 μg/ml, Molecular Probes, Holland). The fluorescence was examined by using a Zeiss Axioplan 2 microscope (Carl Zeiss AB, Sweden) equipped with a CCD (C4880, Hamamatsu Photonics, Japan).

### Tissue Preparation

Dissected porcine hearts (260-390 g) were transported from local slaughterhouse to the laboratory in ice-cold Krebs-Henseleit solution (119 mM NaCl, 23.8 mM NaHCO₃, 3 mM KCl, 1.14 mM NaH₂PO₄, 1.63 mM CaCl₂, and 16.5 mM glucose). Left anterior descending coronary artery and great cardiac vein were isolated. Part of each blood vessel was used immediately after Isolation for contraction assays while part of it was kept frozen in liquid nitrogen for the membrane preparation. In some experiments blood vessels in which endothelium was mechanically removed were used. The same procedure was used also for the preparation of human umbilical blood vessels; umbilical cords were obtained from Obstetric Clinic of Ljubljana Clinical Center, Slovenia.

### Blood Vessel Contraction Measurement

The measurements were performed as already described (20). Rings (5 mm wide) of blood vessels were cut and mounted into 10 ml tissue chamber filled with Krebs-Henseleit solution (37° C.; pH=7.4) which was oxygenated with the mixture of ⁹⁵% O₂ and 5% CO₂. Initial tension of 50 mN was applied and after equilibration 30 mM KCl was added to obtain stable Isometric contraction. The presence of endothelium was verified with substance P. After washing out of KCl and substance P and after the equilibration of the system was restored, test peptide was added into the tissue chamber and blood vessel contraction was recorded.

### Overexpression of G-Proteins in Sf9 Cells

Baculovirus transfer vectors harboring the genes for Gsα, Giα1 and β1γ2 were kindly provided by Dr. Tatsuya Haga (University of Tokyo, Tokyo, Japan). The cDNA for bovine Gsα (21) in pVL1392, bovine Giα1 (22) In pVL1392 and bovine β1γ2 (23) in pVL1393 were cotransfected with linearized baculovirus DNA (Pharmingen, San Diego, Calif., USA), and the resulted virus stocks were subjected to one round of plaque purification before generation of high-titer virus stocks. In the expression procedure, Sf9 cells at density of 2 million cells/ml in suspension culture were Infected with a ratio 2:1 of a versus βγ. Cells were harvested 48 h post-infection, washed in PBS and stored at −70° C. until membrane preparation.

### Membrane Preparation

Frozen pieces of blood vessels were mechanically pulverized. Subsequently membranes were obtained according to the protocol of McKenzie (McKenzie, F. R. Signal Transduction (Milliqan, E., Ed.), Oxford University Press, Oxford, N.Y., Tokyo. (1992)) with minor modifications. Until used, they were kept frozen in the concentration of 1-2 mg protein/ml as determined by the method of Lowry (Lowry, O. H., Rosenbrough, N.Y., Farr, A. L. & Randall, R. J. J. Biol. Chem. 193, 265-275 (1951)). The same procedure (with the exception of pulverization of tissue) was used also for the preparation of membranes from Sf9 cells which overexpressed the G-proteins of different types; protein concentrations of these membrane preparations were between 0.2 and 0.4 mg/ml.

### The Rate of GTPγS Binding

The binding to G-proteins from blood vessel membranes was followed as described by McKenzie (McKenzie, F. R. Signal Transduction (Milligan, E., Ed.), Oxford University Press, Oxford, N.Y., Tokyo. (1992)). Briefly, the membranes (final protein concentration in the assay mixture was 0.05 mg/ml) were incubated for 3 min in the absence and presence of peptides in different concentrations with 0.5 nM [³⁵S]GTPγS at 13° C. In TE-buffer (10 mM Tris-HCl, 0.1 mM EDTA), pH 7.5. The unbound [355]GTPγS was removed by rapid filtration of the reaction mixture through Millipore GF/C glass-fiber filters under vacuum. The remaining radioactivity contained in the filters was determined in the LKB 1214 Rackbeta liquid scintillation counter. Blank values were determined by replacing the membrane preparations with buffer.

### Measurement of GTPase Activity

The measurements were performed radiometrically according to Cassel and Selinger (Cassel, D. & Selinger, Z. (1976) 452(2), 538-51. 452, 538-551 (1976, with the modifications suggested by McKenzie (McKenzie, F. R. Signal Transduction (Milligan, E., Ed.), Oxford University Press, Oxford, N.Y., Tokyo. (1992)). To the diluted membranes obtained from Sf9 cells with the overexpressed G-proteins of different types (final protein concentration in the assay mixture was 0.01 mg/ml) the Ice cold reaction cocktail containing ATP (1 mM), 5-adenylylimido-diphosphate (1 mM), ouabain (1 mM), phosphocreatine (10 mM), creatine phospho-kinase (2.5 Units/ml), dithlothreltol (4 mM), MgCl₂ (5 mM), NaCl (100 mM), and trace amounts of [γ-³²P]GTP to give 50.000-100.000 cpm in an aliquot of the reaction cocktail (with the addition of cold GTP to give the required 0.5 μM total concentration of GTP) was added. Incubation medium was standard TE-buffer, pH 7.5. Background low-affinity hydrolysis of [γ-³²P]GTP was assessed by incubating parallel tubes in the presence of 100 μM GTP. Blank values were determined by the replacement of membrane solution with assay buffer. GTPase reaction was started by transferal of the reaction mixtures to 30° C. water bath for 20 min. Unreacted GTP was removed by the 5% suspension of the activated charcoal in 20 mM H₃PO₄. The radioactivity of the yielding radioactive phosphate was determined in Packard 3255 liquid scintillation counter.

Curve fittings and other calculations as well as graphical presentations of the results were done by using a Prism computer program (GraphPad Software Inc., USA).

### Results

### Cellular Internalisation of Peptides

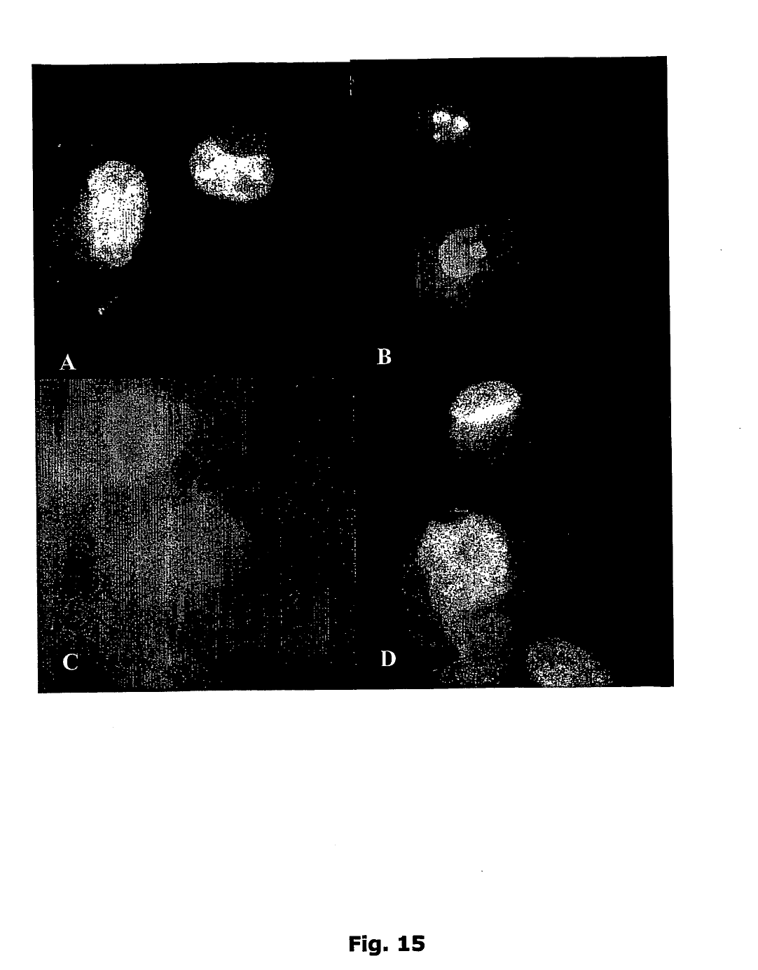

Blotinylated M511 internalized into living cells at both 4° C. and 37° C., as judged by indirect immunofluorescence. The peptide translocates in a temperature-independent manner into Bowes melanoma cells and therefore, the main mechanism of uptake could not be endocytosis. The peptide localized preferentially in nuclei (FIG. 15A) but also in the cytoplasm. Scrambled analogue of M511 (also biotinylated) was found to internalize into Bowes cell yielding a similar cellular localization and temperature dependence with M511 (FIG. 15B), with, however, slightly lower efficiency. Penetratin a well-studied cell-penetrating peptide (12-14) was used as positive control (FIG. 15D).

### Blood Vessel Contraction

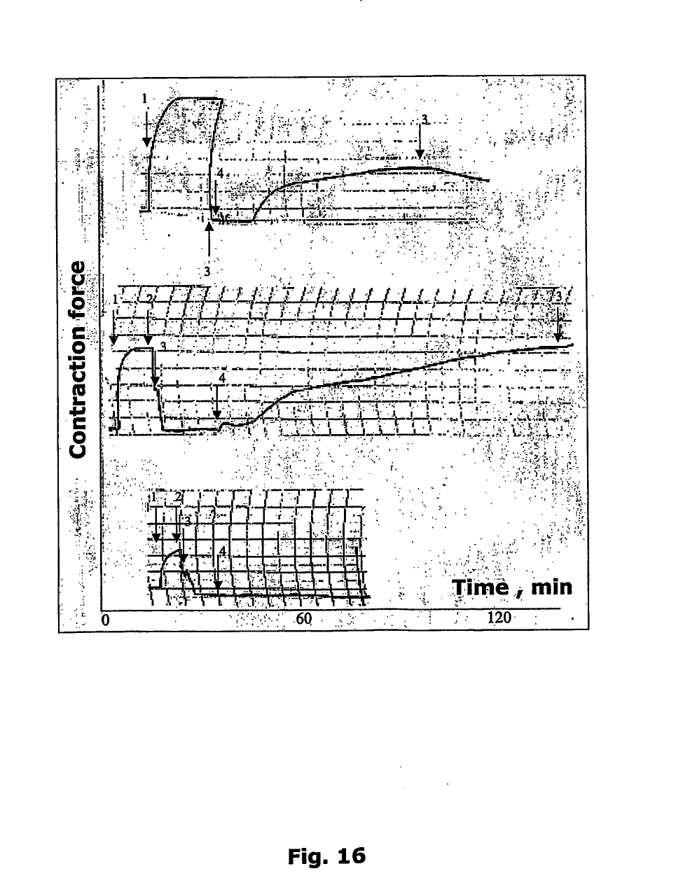

It was observed that M511 and blotinylated M511 act as powerful contractors of blood vessels. As seen in upper panel of FIG. 16, M511 at 16 μM concentration promotes intense and long-lasting contraction of porcine left anterior descending coronary artery. The strength of contraction is comparable to the effect of 30 mM K⁺ that is approximately 50% of maximal effect of potassium ion. With higher concentrations the maximal effect approaches the effect of 90 mM KCl that is generally considered to be a maximal attainable contraction effect. Contrary to the contractory effect of potassium, the effect of M511 could not be terminated by washing the contractor out of assay solution. Furthermore it showed a concentration dependent 5 to 15 min lag-period after addition of M511 before contraction occurred. Results (FIG. 16 middle panel) also show that blood vessel endothelium is not essential for the effect of M511.

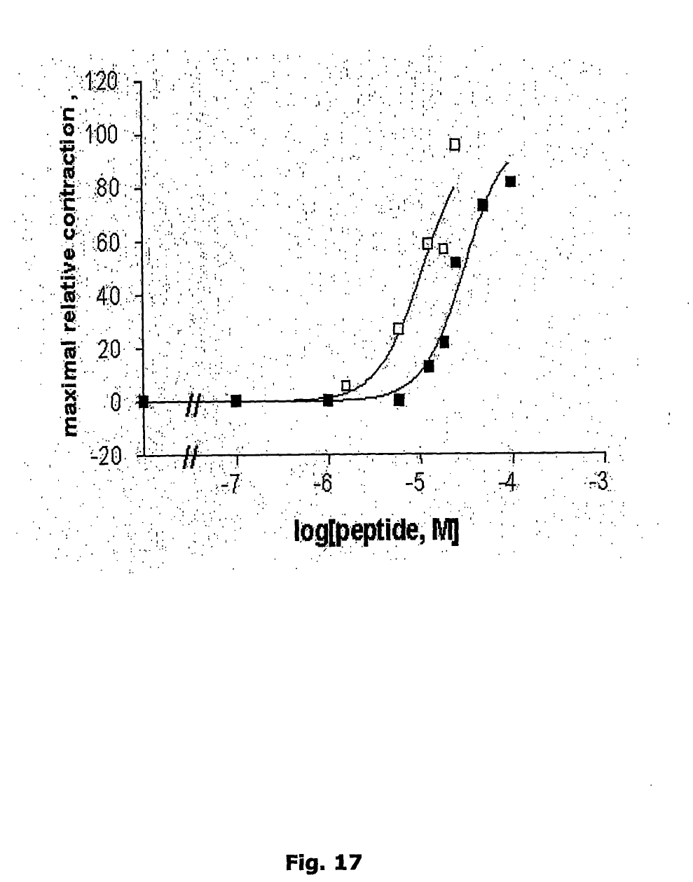

Qualitatively the same but quantitatively more pronounced blood vessel contraction as shown in FIG. 16 for M511 was observed also with biotin coupled M511 (diagram not shown). Concentration dependency of the maximal contraction force of porcine left anterior descending coronary artery exerted by M511 and biotin labeled M511 is shown in FIG. 17. Scrambled M511 under same circumstances as M511, did not cause any contraction (FIG. 16 bottom panel).

### GTPγS Binding

As presented in FIG. 17, the rate of binding of GTPγS to the membranes obtained from the porcine left anterior descending coronary artery was dose-dependently increased in the presence of M511, which was even more pronounced in the presence of blotinylated M511. The upper plateau of the effect was not obtained since the experimental points in the presence of peptides in concentrations over 500 μM could not be used. It is obvious that this effect is sequence specific for M511 (and also blotinylated M511) since the scrambled peptide is not active. Principally the same results were obtained also with membranes prepared from porcine great coronary vein and human umbilical artery (diagrams not shown).

### G-Protein Selectivity

In order to shed some light on the type of G-proteins that were affected by M511, the inventors used membranes from Sf9 cells overexpressing G-proteins of different types and measured GTPase activity of these membranes in the absence and presence of M511 (100 μM). The results summarized in FIG. 17 show small but significant activation of Gᵢ and Gₒ and, interestingly, also moderate inhibition of G₁₁; Gₛ type of G-proteins seems not to be affected. These findings are in accordance with the suggestion that ATLA receptors function via inhibition of adenylyl cyclase (activation of Gᵢ/Gₒ) and via modulation of phosphoinositide metabolism, most probably through pertussis toxin insensitive G-proteins (G_{q} and G₁₁). Indeed was demonstrated regulation of G₁₁ by M511, however, not by activation of this type of G-proteins but rather by their Inhibition.

### Effect of Sar¹-Thr⁸-Angiotensin II

Additionally, the inventors could also prove that angiotensin II antagonist Sar¹-Thr⁸-angiotensin II was able to revert the contraction effect of anglotensin II on the porcine left anterior descending coronary artery, but was unable to affect the contraction of blood vessels induced by M511. This is a strong indication that anglotensin 11 and M511 are not acting via the same contraction mechanism and corroborates the finding that M511 does not activate angiotensin receptor but that it probably binds directly to G-proteins and induces activation of phospholipase C.

Furthermore, it is most likely that in contracting the blood vessels, M511 is more efficient than anglotensin II, since it induced about 40% higher contraction force compared to that induced by 30 mM KCl, while the effect of angiotensin was almost the same as the effect of 30 mM KCl. 1 μM anglotensin and 100 mM M511 were used in order to achieve the maximal effect of both ligands, as is shown in FIG. 20.

### CONCLUSIONS

M511 is a peptide corresponding to rat AT1A receptor positions 304327 (Table 7). As FIG. 15 demonstrates, biotinylated M511 penetrates into human melanoma cell-line Bowes similarly to a well-studied cell-permeable peptide Penetratin. Moreover, results obtained by porcine artery and vein vessel contraction, confirm Internalization of M511 and suggest, that the Internalized peptide may compete with native receptor and affect its signaling pathway. Interestingly scrambled blotinylated M511 internalize as well into Bowes cells, but does not cause contraction in artery or vein vessel. The observed lag-period in muscle contraction studies could be interpreted as time required for the penetration of sufficient amount of the peptide into the cell and the shortening of lag-period with the increasing concentration of peptides, as well as the Inability to terminate the contraction by washing M511 from the assay solution, would be in accordance with its intracellular action. Virtually Identical effect of M511 in porcine artery and great cardiac vein and human umbilical artery, illustrate that this effect is not restricted only to arteries and Indicating its general nature in blood vessels of different tissues.

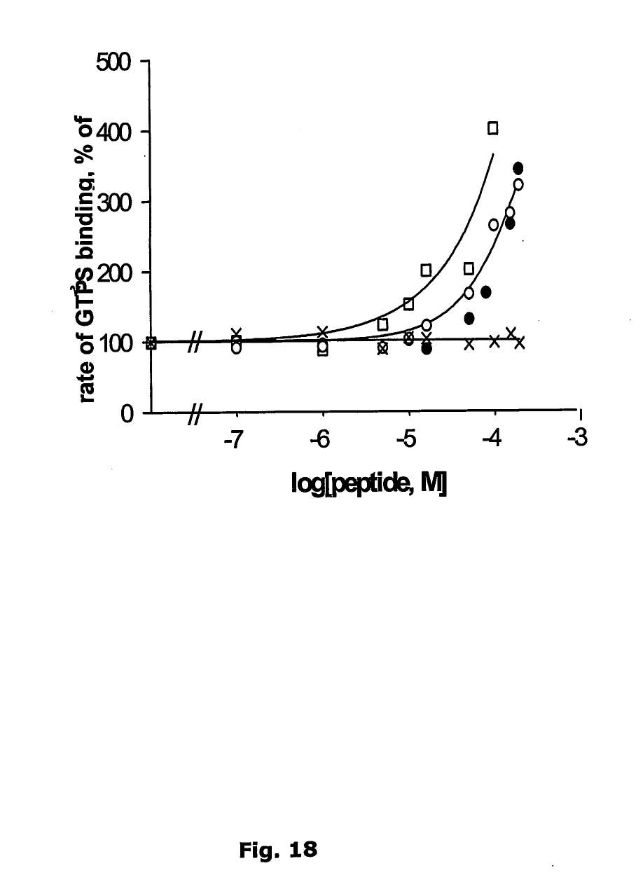

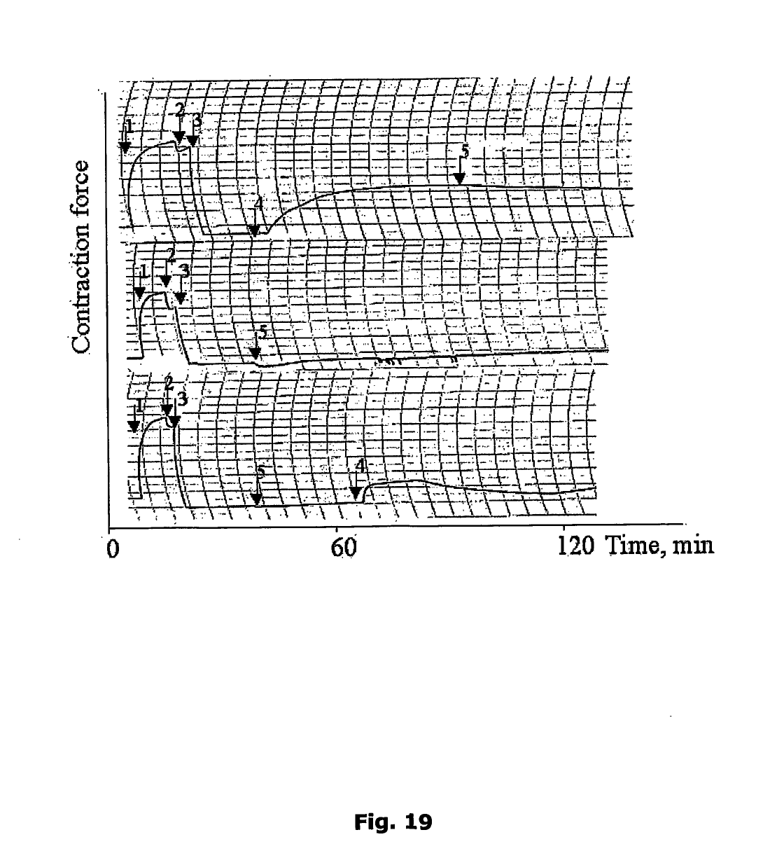

It is well known that AT1A as a member of 7-transmembrane receptors, is coupled to G-proteins via interaction of G subunit and the intracellular parts of the receptor. Increased rate of GTPγS binding (FIG. 18) proves the involvement of G-proteins in the process of blood vessel contraction induced by M511 and corroborate the idea that M511 uncouples G-proteins from the AT1A receptors. The presented results match well with the effect of the peptides on blood vessel contraction (see FIGS. 16 and 17).

In order to elucidate which G-proteins might be involved in the action of M511, GTPase activity in membranes overexpressing different types of G-proteins was measured. Slight activation of Gᵢ/Gₒ and no effect on Gₛ is in good accordance with previous studies.

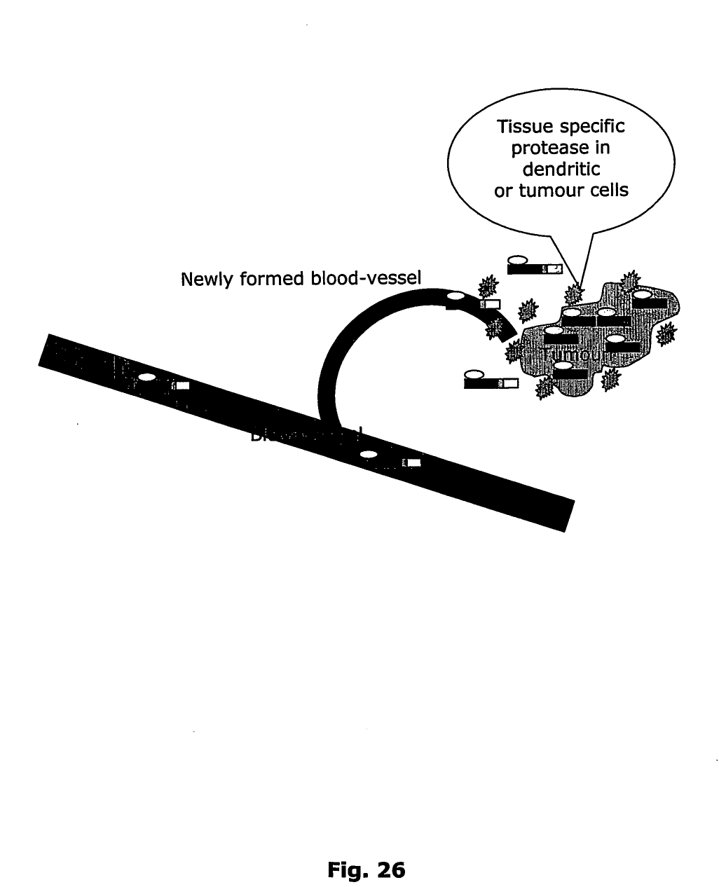

The inventors further inspected the mechanism of M511 action by using phospholipase C inhibitor U73122. As seen in FIG. 26A, U73122 at 30 μM concentration did not affect tonus of the porcine left anterior descending coronary artery (middle panel), and also did not modify blood vessel contraction induced by 16 μM concentration of M511 after posterior administration (upper panel), but it substantially decreased (for more than 50%) the effect of M511 when added 30 minutes prior to M511 administration (lower panel). This indicates that blood vessel contraction by M511 is mediated via phosphoinositole phosphate mechanism, as expected.

Another intriguing discovery is amplification of the effect of the peptide on blood vessel contraction via biotinylation. It can be proven by principally same effect on GTPγS binding rate (FIG. 18). GTPγS binding rate demonstrates also that N-terminal blotin on M511 does not induce a parallel signaling cascade, leading to contraction, but rather amplifies interactions between G-proteins and the peptide.

In conclusion, M511 (and even more remarkably, biotinylated M511) seems to be a powerful vasoconstrictor that successfully penetrates the cells and functions via uncoupling of AT1A receptors from Gₗ and Gₒ proteins, and possibly also via Inhibiting G_{q} proteins. As such it is an interesting drug candidate aimed against chronical hypotension and possibly also migraine. Its potential disadvantage is relatively high concentration required for the blood vessel contraction but its advantage could be its spontaneous internalization into the cells and Its long-lasting action. Besides that it gives a new quality for angiotensin studies.

### Summary of Results

The inventors studied angiotensin and signal transduction via its receptors. By Investigation of cell-penetrating peptides, they succeeded to design a peptide that penetrates cell membranes and Induces ex vivo intracellular signaling cascade similarly to the AT1A type of angiotensin receptor. The peptide, M511, corresponding to the fragment 304-327 of rat AT1A receptor, was found to Internalize into Bowes cells in a temperature Independent manner. This observation was confirmed by contractions of blood vessels from different origins. Induction of long lasting contraction of porcine coronary artery vessel and vessels from several other origins, after concentration dependent lag-period was assigned for the solution of the peptide. In order to discover principles of this action, influence on GTPγS binding rate and Gα-subtype selectivity of the peptide were measured. Results indicate, that the M511 peptide interacts with same selectivity to G-protein subtypes as agonist activated ATLA receptor, activating/inhibiting them. Down-regulation of blood vessel contraction by U73122 indicates that the further pathway involves phospholnositole phosphate system and stimulates phospholipase C for M511 was observed also with biotin coupled M511 (diagram not shown). Concentration dependency of the maximal contraction force of porcine left anterior descending coronary artery exerted by M511 and blotin labeled M511 is shown in FIG. 17. Scrambled sequence M511 under same circumstances as M511, did not cause any contraction (FIG. 16 bottom panel).

### Example 9

### Combining the Effects of PEI and TP10 and/or YTA-2

In this study the inventors developed a new gene delivery system based on already existing PEI protocols. By combining the effects of PEI and TP10 or YTA-2 together substantially higher transfection ratios were achieved than with PEI only. The approach was to crosslink TP10 to transfection reagent. Thereafter PEI of common transfection protocol was replaced by CPP modified one, and no more changes in protocol were done. Under optimal conditions, the results postulate a significant improvement in gene delivery compared to other systems.

### Materials and Methods

### Synthesis and Purification of TP-10 and YTA-2

The peptides were synthesized in a stepwise manner on an Perkin Elmer/Applied Biosystem Model 431A peptide synthesizer, using t-Boc strategy according to protocol described previously (Langel, Land et al. 1992). Cysteine or glutamic acid was coupled manually. TP10 sequence is given in (Pooga, 1998, FASEB J.). Cysteine or glutamic acid was coupled manually. TP10 sequence is given in Pooga, 1998, FASEB J. YTA-2 sequence is given in table 5 and listed as SEQ.ID.NO.31913. Prior conjugating peptides to PEI, they were purified on a reversed phase HPLC (Gynkotek) C18 column with AcN/H₂O gradient, and analyzed using a MALDI-TOF Mass spectrometer (Applied Biosystems model Voyager STR). The mass values obtained matched calculated values.

### PEI Modifications

### PEI Modifications

Conjugation of TP10 or YTA-2 to PEI was done in two alternative ways. In the first case, a cysteine was coupled to Lys7 side chain of TP10 or N-terminus of YTA-2. PEI (1 mg/ml, MW: 60 kDa, Aldrich) was treated with bifunctional crosslinker succinimidyl trans-4-(maleimidylmethyl)cyclohexane 1-carboxylate (SMCC) at concentratios needed for different TP10/PEI molar ratios. In the second case, glutamic acid was coupled to the N-terminus of TP 10 and was further covalently coupled to PEI (MW: 25 kDa, Sigma) using BOP generated Hobt esters. The calculated molecular weight of formed complexes was confirmed by MALDI TOF mass-spectrometry.

### Cell Culture

Murine fibroblasts C3H 10T1/2, mouse neuroblastoma N2A cells and COS-7 cells were grown in 10 cm petri dishes in Dulbecco's Modified Eagle's Media (DMEM) supplemented with 10% fetal calf serum (FCS), 2 mM L-Glutamine, 100 U/ml penicillin and 0.1 mg/ml streptomycin at 37° C. in a 5% CO₂ atmosphere. The cells were seeded and replated every fifth day. COS-7 and 10T1/2 cells were trypsinized when seeded while the N2A cells were suspended by mechanical force by adding media to the cells. Before starting the experiments, the cells were grown to confluency and then seeded and diluted two times in media before adding to 24-well plates (approximately 60 000 cells/well).

### Propagation of Plasmids

pEGFP-N1, pEGFP-C2 (both Clontech), pGL3-Promoter Vector (Promega) and pRL-CMV Vector (Promega) plasmids were propagated in replication competent E. coli, according the protocol suggested by the manufacturer. The propagated plasmids were purified using Qiagen midiprep (Qiagen) and then applied on agarose gel in order to estimate the purity and concentration of plasmid. The concentrations were then finally determined by ON spectrometry for each plasmid.

### Transfection

Transfection of COS-7 and N2A cells and murine fibroblasts C3H 10T½. PEI solution was dialysed by dialysis membrane (MW-cutoff 15 kDa) in order to remove smaller PEI fragments that can be toxic to the cells. Thereafter TP10 was crosslinked to PEI as described above. The PEI and/or CPP-PEI stock solutions (1 mg/ml) were diluted so that the same volume of solution could be taken for each N/P ratio experiment. 10 μl of plasmid (0.05 μg/μl) and 10 μl of transfection reagent were mixed in a 96 well plate, each experimental point separately. The mixture was incubated 15 min at room temperatures. Meanwhile media of 24 hours prior seeded cells was changed to fresh (250 μl/well in 24 well plate). After incubation of plasmid and PEI was finished, 20 μl of the conjugate was added to cells. Transfection was carried out for 3 h at 37° C., followed by media change. Efficiency of transfection was measured 24 or 72 h after transfection.

### Fluorescense Microscopy

The level of GFP expression was investigated by an inverted fluorescence microscope (Zeiss Axiovert 200 equipped for fluorescence microscopy, or Olympus IMT2 inverted microscope with RFL-1 fluorescence device), 1-3 days after transfection.

### GFP Quantification

For GFP quantification in murine fibroblasts C3H 10T1/2 cells were lysed and the lysate was exitated at 485 nm, and UV emission was measured at 527 nm using Labsystems. Fluoroscan Ascent CF instrument (Labsystems, FI).

Cells transfected by renilla or firefly luciferase gene were lysed 36 hours after transfection using passive lysis buffer (Promega). Samples were freeze-thawed once and luciferase activity was measured on the same or next day. Protocol of dual luciferase kit, recommended by manufacturer (Promega) was used on Victor (Wallac, Finland) luminometer. Obtained luciferase activities were transformed into activity per mg of total protein concentration. Determination of total protein concentration was performed by Bradford method.

In vivo Transfection of Chicken Embryo

Eggs with developing chicken embryos (3 days) were opened and SMV-lacZ reporter genes were injected on embryo near neural tube. After 48 h embryos were removed and fixed for 1-2 hours in 2% paraformaldehyde, 0.25% glutaraldehyde, washed with PBST and stained with staining solution (9 ml spermidine, 1 ml of 2% Xgal in DMF 0.5 ml of 165 mg/ml K-ferricyanide, 0.45 ml 210 mg/mL K-ferrocyanide) until blue color developed (2 h).

### Results

### CPP-PEI and GFP

TP10-PEI as well as YTA-2-PEI constructs mediated considerably higher levels of GFP transfection compared to unmodified PEI protocol at all tested concentrations. The most significant increase was achieved at concentrations below 1 μg TP10-PEI per well. At higher concentrations, the difference between modified and unmodified PEI was still significant, but not so drastic. FIG. 21A a, b and c. demonstrate the effect of unmodified PEI at concentrations of 0.25, 0.5 and 1.0 μg per well, respectively. d, e and f in the same figure correspond to TP10 modified PEI at same concentrations. In a parallel study, murine fibroblasts C3H 10T1/2 were used instead of N2A cells. The GFP expression was determined up to 14 times higher (ratio of PEI-TP 0.4 μg vs. DNA 1.2 μg) as with PEI alone under given experimental conditions. In fluorescent microscopy, the number of eGFP expressing cells was significantly higher in the samples transfected with PEI-TP.

### TP10-PEI and Luciferase

Under optimal conditions, in full growth media, TP10 modified PEI (TP10/PEI molar ratio 5) mediated about an 100% Increase in luciferase transfection efficiency (se FIG. 21B) Under nonoptimal conditions no significant positive effect was observed.

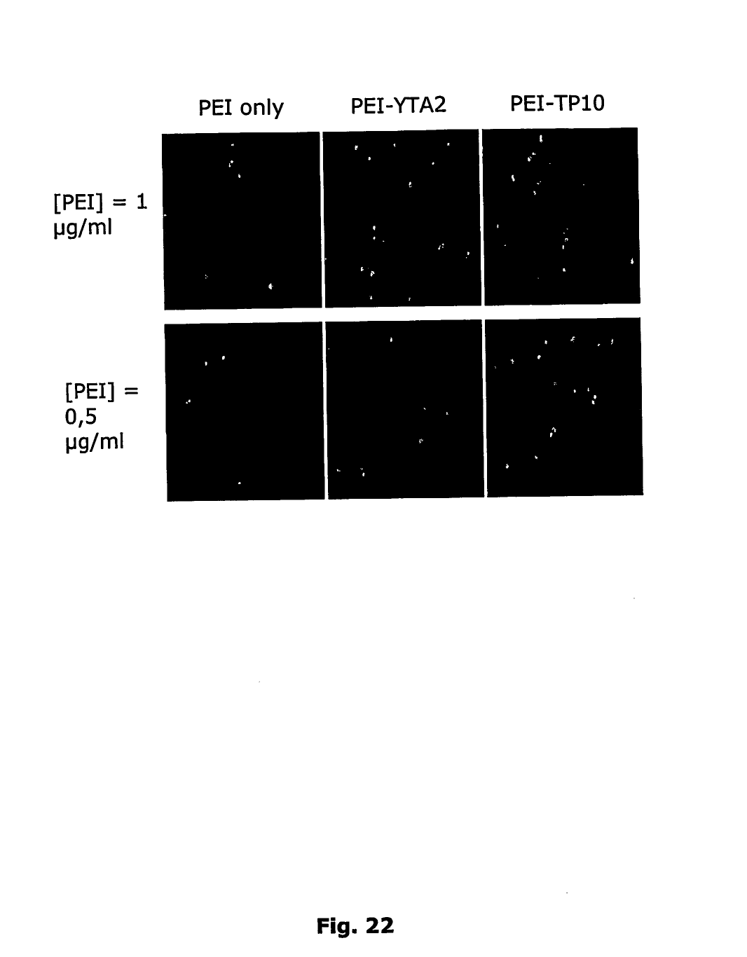

FIG. 22 shows transfection of mouse neuroblastoma N2A cells with unmodified polyethylenimine (PEI) or chemically crosslinked PEI and cycteinyl YTA2 or TP10 peptide. Two different concentrations of PEI or peptidyl-PEI were tested: 1 or 0.5 μg per 1 ml of media. Transfection was performed for 3 h in serum-free growth media (DMEM) and photos were taken 48 h after transfection. Improvement of transfection was registered with application of both, YTA2 and TP10 peptides. YTA2-PEI and PEI-TP10 transfected 2 times more cells than unmodified PEI, and also the expression level in transfected cells was higher.

### TP10/PEI Molar Ratio

Four different TP10/PEI ratios in the range of 1 to 100 molecules of TP10 per 1 polycationic molecule were tested. Results showed that the delivery yield is depending on the ratio. 5 TP10 per one PEI was found to be the best ratio, followed by 20. The most optimal ratio is probably between 5 and 20. Ratios 1 and 100 had lowest effect.

In vivo Transfection of Chicken Embryo

When SMV-lacZ alone or in complex with PEI was applied to the embryo, no expression of the reporter gene was observed. If SMV-lacZ was complexed with PEI-TP10 the reporter gene was expressed in a neural tube region as detected by specific staining. The B-galactosidase staining was strong and was distributed with equal intensity all over the neural tube region.

### Example 10

### Positional Scanning of CPP within Human 7™ Receptors

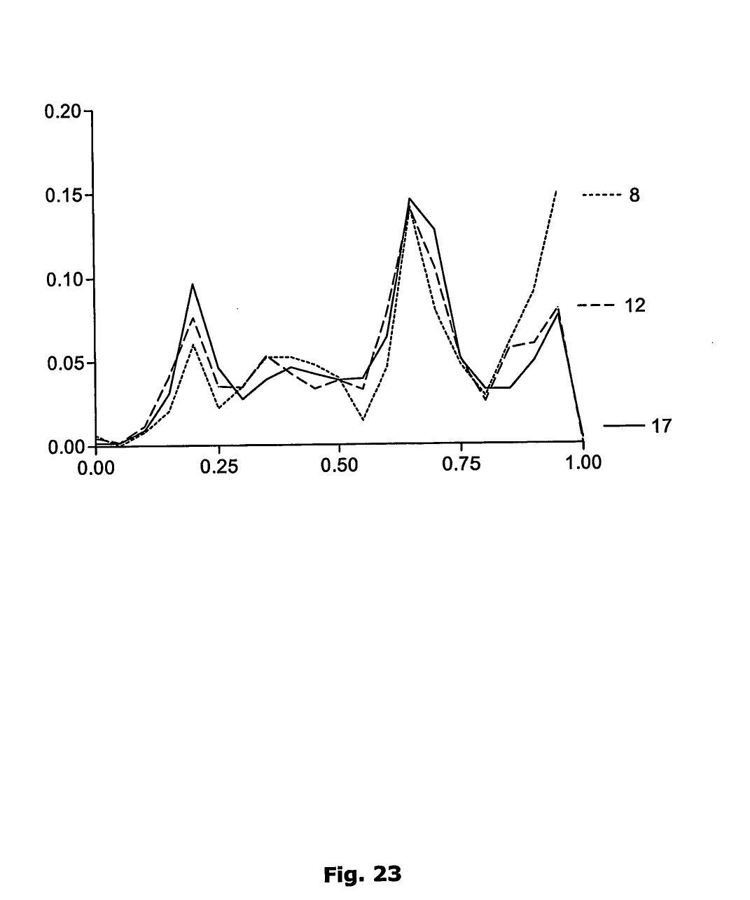

Human 7™ receptor sequences were downloaded from the swissprot/trmbl databases. The sequences were searched for CPPs of the indicated length (8, 12 and 17 aa long). The position of the start of the CPP in the protein was divided by the total length of the protein, plotted against the frequency of occurrence (fraction of CPPs). Here, a search window in the size of 17 aa produced the most hits. The four peaks in evidence correspond to the four intracellular parts of the 7TM receptors, with the largest peak corresponding to the third internal loop (IC3). It can be noted that the CPP functionality seem to correlate well, both with the topology of the 7™ receptors, as well as with the proposed G protein activation sites. See FIG. 23.

### Example 11

### New Selection Criteria

### Three Graded System

The three grades represent a successive narrowing of the descriptor interval. The performance of the grades can be seen from table 10. Principally, the higher the grade, the lower the chance that a predicted CPP is a “false” positive. However, the chance that a CPP is missed also increases.

### New “Descriptors”

Two additional descriptors are introduced: Bulkha being the number of non-hydrogen atoms (C, N, S and O) in the side chains of the amino acids, and hdb standing for the number of accepting hydrogen bonds for the side chains of the amino acids.

TABLE 10
New descriptors:

| Amino acid | Bulkₕₐ | hdb |
|---|---|---|
| A | +1 | 0 |
| C | +2 | 0 |
| D | +4 | −4 |
| E | +5 | −4 |
| F | +7 | 0 |
| G | 0 | 0 |
| H | +6 | −2 |
| I | +4 | 0 |
| K | +5 | +3 |
| L | +4 | 0 |
| M | +4 | 0 |
| N | +4 | 0 |
| P | +3 | 0 |
| Q | +5 | 0 |
| R | +7 | +5 |
| S | +2 | −1 |
| T | +3 | −1 |
| V | +3 | −0 |
| W | +10 | +1 |
| Y | +8 | 0 |

The supplemented selection criteria uses Z_{Σ1}, Z_{Σ2}, Z_{Σ3}, Z_{ΣBulkha} and net hydrogen bond donation (hdb) and average hdb. Bulkha is calculated as number of atoms in the side chain of the amino acids not counting the hydrogens e.g. for CH₂CH₂OH (serine) Bulkₕₐ=2*C+1*O=3. hdb is calculated as the donated hydrogen bonds-accepted hydrogen bonds of the side chains. E.g. N—H donates and C═O accepts.

The three grades represent a successive narrowing of the descriptor interval. The performance of the grades can be seen from table 11.

TABLE 11
Correctly predicted, in %, for the 3 grades

| | Grade is | | |
|---|---|---|---|
| | >0 | >1 | >2 |
| Positives | 100 | 72 | 41 |
| Negative | 50 | 61 | 82 |
| Unrelated | 80 | 90 | 100 |

Positives correspond to the CPP training-set, negatives corresponds to the non functional CPP analogues training-set and unrelated corresponds to hormone training-set.

TABLE 12
“training sets”.

| | Non-functional-CPP-analogues | |
|---|---|---|
| CPP training-set | training-set | Hormone training-set |
| GWTLNSAGYLLGKINLKALAALAKKIL | GWTLNSAGYLLGKFLPLILRKIVTAL | QNLGNQWAVGHLM |
| RQIKIWFQNRRMKWKK | LLGKINLKALAALAKKIL | RPPGFSPFR |
| KLALKALKALKAALKLA | LNSAGYLLGKALAALAKKIL | LYGNKPRRPYIL |
| LLIILRRRIRKQAHAHSK | LNSAGYLLGKLKALAALAK | GWTNLSAGYLLGPPPGFSPFR |
| AGYLLGKINLKALAALAKKIL | GWTLNSAGYLLGKINLKAPAALAKKIL | GWTLNSAGYLLGPHAI |
| FLGKKFKKYFLQLLK | LLKTTALLKTTALLKTTA | HDEFERHAEGTFTSDVSSYLEGQAA |
| | | KEFIAWLVKGR |
| GRKKRRQRRRPQ | LLKTTELLKTTELLKTTE | WSYGLRPG |
| RRRRRRRRR | GRKKRRQPPQC | TIHCKWREKPLMLM |
| GWTLNSAGYLLGKINLKALAALAKKLL | FITKALGISYGRKKRRQC | FVPIFTHSELQKIREKERNKGQ |
| GWTLNPAGYLLGKINLKALAALAKKIL | QNLGNQWAVGHLM | AGCKNFFWKTFTSC |
| GWTLNPPGYLLGKINLKALAALAKKIL | RPPGFSPFR | CYFQNCPRG |
| LNSAGYLLGKINLKALAALAKKIL | LYGNKPRRPYIL | |
| GWTLNSAGYLLGKLKALAALAKKIL | GWTNLSAGYLLGPPPGFSPFR | |
| RRWRRWWRRWWRRWRR | GIWFAYSRGHFRTKKGT | |
| GWTLNSKINLKALAALAKKIL | LRKKKKKH | |
| LNSAGYLLGKLKALAALAKIL | VATIKSVSFYTRK | |
| AGYLLGKLKALAALAKKIL | KKKQYTSIHHGVVEVD | |
| KLALKLALKALKAALK | RQIKIFFQNRRMKFKK | |
| KLALKLALKAWKAALKLA | KKLSECLKRIGDELDS | |
| KITLKLAIKAWKLALKAA | PVVHLTLRQAGDDFSR | |
| KALAKALAKLWKALAKAA | EILLPNNYNAYESYKYPGMFIALSK | |
| KALKKLLAKWAAAKALL | IAARIKLRSRQHIKLRHL | |
| KLAAALLKKWKKLAAALL | | |
| LKTLATALTKLAKTLTTL | | |
| KALAALLKKWAKLLAALK | | |
| KLALKLALKALQAALQLA | | |
| KLALQLALQALQAALQLA | | |
| QLALQLALQALQAALQLA | | |
| LLKKRKVVRLIKFLLK | | |
| RLIKTLKTLLQKRKTL | | |
| NAKTRRHERRRKLAIER | | |
| LLIILRRPIRKQAHAHSK | | |
| LLIILRARIRKQAHAHSK | | |
| LLIILRRRIRKQAHAHSA | | |
| TRRNKRNRIQEQLNRK | | |
| GGRQIKIWFQNRRMKWKK | | |
| MGLGLHLLVLAAALQGAKKKRKV | | |
| RKKRRQRRR | | |
| GRKKRRQRRRPPC | | |
| GRKKRRQRRRC | | |
| GRKKRRQRRPPQC | | |
| RQPKIWFPNRRMPWKK | | |
| RQIKIWFPNRRMKWKK | | |
| TRQARRNRRRWRERQR | | |
| KMTRAQRRAAARRNRWTAR | | |
| RVIRVWFQNKRCKDKK | | |
| RKSSKPIMEKRRRAR | | |
| YGRKKRRQRRRPPLRKKKKKH | | |
| RQIKIWFQNRRMKWKKLRKKKKKH | | |
| VQAILRRNWNQYKIQ | | |
| MAQDIISTIGDLVKWIIDTVNKFTKK | | |
| KRPAATKKAGQAKKKKL | | |
| RRRRNRTRRNRRRVR | | |
| TRRQRTRRARRNR | | |
| MDAQTRRRERRAEKQAQWKAAN | | |
| TAKTRYKARRAELIAERR | | |
| RQGAARVTSWLGRQLRIAGKRLEGR | | |
| SK | | |
| RQGAARVTSWLGRQLRIAGKRLEGR | | |
| GAARVTSWLGRQLRIAGKRLEGRSK | | |
| RVTSWLGRQLRIAGKRLEGRSK | | |
| SWLGRQLRIAGKRLEGRSK | | |
| GRQLRIAGKRLEGRSK | | |
| KCRKKKRRQRRRKKLSECLKRIGDE | | |
| LDS | | |
| KCRKKKRRQRRRKKPVVHLTLRQAG | | |
| DDFSR | | |
| AAVALLPAVLLALLAPVQRKRQKLMP | | |
| RRRRRRWGRWGRWGRWGRWGR | | |
| WGRPKKKRKV | | |
| ALWMTLLKKVLKAAAKAALNAVLVG | | |
| ANA | | |
| ALWKTLLKKVLKA | | |
| PKKKRKVALWKTLLKKVLKA | | |
| RQARRNRRRALWKTLLKKVLKA | | |
| RQARRNRRRC | | |
| RRLSYSRRRF | | |
| RGGRLSYSRRRFSTSTGR | | |
| YGRKKRRQRRRSVYDFFVWL | | |
| YGRKKRRQRRRGTSSSSDELSWIIE | | |
| LLEK | | |
| IVIAKLKA | | |

Non-natural sequences listed as SEQ. ID. NO. 31923-31940:

| | |
|---|---|
| Evo162 | KTVLLRKLLKLLVRKI |
| Evo163 | KIIKRLIVVRLITLVIK |
| Evo164 | LLKLKLLAILKIKLIV |
| Evo83 | KLIRKRLI |
| Evo86 | RLIKRLIK |
| Evo86 dimer | (RLIKRLIKC)₂ |
| Evo165 | LLKKRKVVRLIKFLLK |
| Evo165 analogue | LLKKRKVVRLIKQKQK |
| Evo165 analogue | LLKKRKVRLIKQKQK |
| Evo165 analogue | LLKKRKVVRLIKAHSK |
| Evo165 analogue | LLKKRKVRLIKAHSK |
| Evo165 analogue | LLKKRKVVRLIKVRK |
| L-407-Abz | LKLLYKNKLLKYNLKamide |
| L-408-Abz | KLFKYKKLKRYFYLQKamide |
| L 409-Abz | YKRLSLVKRLIKamide |
| Evo165-B | Biotin-LLKKRKVVRLIKFLLKamide |
| Evo165 amide | LLKKRKVVRLIKFLLKamide |

As described previously for the first set of selection criteria, the values for the peptide are averaged (divided by number of amino acid residues in the peptide). Z_{Σ1}, Z_{Σ2}, Z_{Σ3}, Z_{ΣBulkha}, Z_{Σhdb}

The Intervals for the Different Grades are:

3: Preferred: Z_{ΣBulkha}>3.1 and Z_{ΣBulkha}<8.13 and Z_{Σ1}>−1.25 and Z_{Σ1}<3.52 and Z_{Σ2}>−3.9 and Z_{Σ2}<3.1 and Z_{Σ3}<−0.5 and Z_{Σ3}>−3.51 and Z_{Σhdb}>−0.115 and Z_{Σrhdb}<5.1 and hdb>0 and hdb<84
2: More preferred: Z_{ΣBulkha}>3.2 and Z_{ΣBulkha}<5.9 and Z_{Σ1}>−1.25 and Z_{Σ1}1.92 and Z_{Σ2}>−1.22 and Z_{Σ2}<1.29 and Z_{Σ3}<−0.5 and Z_{Σ3}>−1.94 and Z_{Σhdb}>0.28 and Z_{Σhdb}<2 and hdb>5 and hdb<30
1: Most preferred: Z_{ΣBulkha}>3.2 and Z_{ΣBulkha}<4.8 and Z_{Σ1}>−1.1 and Z_{Σ1}<1.92 and Z_{Σ2}>−1.1 and Z_{Σ2}<0 and Z_{Σ3}<−0.55 and Z_{Σ3}>−1.94 and Z_{Σhdb}>−0.28 and Z_{Σ1}<1.57 and hdb>7 and hdb<25

### Example 12

seICPP

### Introduction

Matrix metallo proteases (MMPs) are Zn²⁺ metallo endopeptidases. The family contains both membrane bound and secreted members of which both catalyse the breakdown of proteins located either on the cell's plasma membrane or within the extracellular matrix (ECM) (Sternlicht-01).

Because MMPs can degrade the ECM, MMP's influence cell migration, remodeling, and inflammatory responses. However, these enzymes may also be involved in the underlying causes of invasive and inflammatory diseases, including cancer, rheumatoid arthritis, multiple sclerosis and bacterial meningitis (Leppert-01).

One characteristic of invasive processes like metastasis and angiogenesis is the degradation of the ECM and basal membranes, which are normally physical barriers to cell migration. MMPs have been linked to the invasive and metastatic behaviour of a wide variety of malignancies, and these enzymes are generally overexpressed in a variety of tumours (Sternlicht-01, Rozanov-01). The number of different MMPs have been found to increase with tumour progression (Hoekstra-01) and correlate to the invasive capacity of certain tumours (Hornebeck 02). Furthermore, expression of MMPs have been correlated to the number of metastatic growth in transgenic mice (Strenlicht-01).

Membrane type MMPs (MT-MMP) such as MMP-MT1 have been strongly implicated in oncogenesis. These enzymes localise to the invasive fronts. The soluble MMPs 1-3 and 9 have also been implicated as agonists of tumourigenesis (Rozanov-01 and Nabeshima-02). MMP-MT1 is also upregulated during endothelial cell induction and migration during anglogensis (Galvez-01). In addition, MMP-MT1, or MMP14 as it is also called, is involved in the activation of proMMP-2 by its “shedase” activity, thereby releasing active MMP-2 at the inasive front (Sounni-02). MMP-2 appears to have an important role in tumour angiogensis (Chen-01).

seICPPs

The concept of selective CPPs are based on the tissue specificity of MMPs enzyme activity. One approach is to use endocytotic uptake for specificity and activation of the CPP by conjugating it to any receptor ligand, such as galanin.

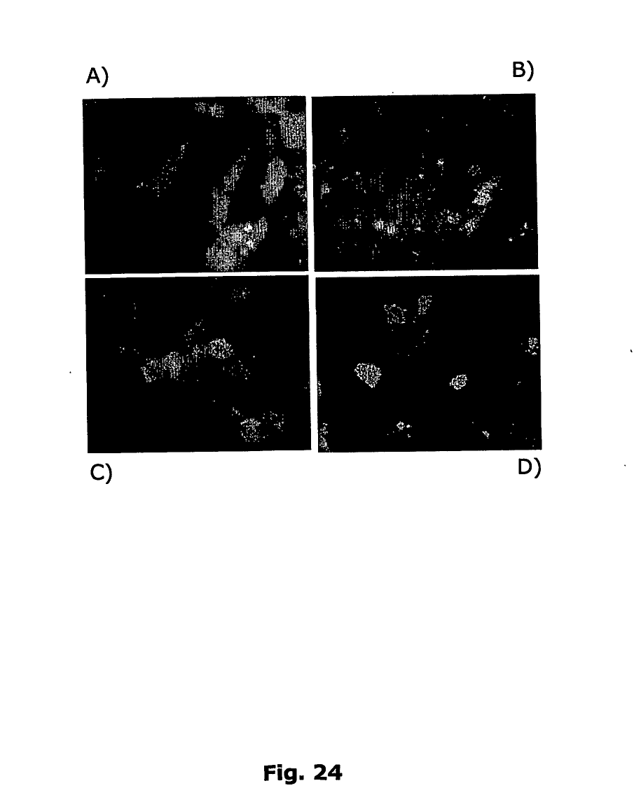

TABLE 13
Sequences of sel CPPs

| Name | Sequence | Uptake | In Cells |
|---|---|---|---|
| YTA-2 | biotin-YTAIAWVKAFIRKLRK-amide | +++ | Lovo, bEnd, |
| SelCPP1 | | | Caco |
| YTA2-ps | biotin-SGESLAY-YTAIAWVKAFIRKLRK-amide | + | Lovo, bEnd, |
| | | | Caco |
| LRSW-1 | LRSWVISRSIRKAA-amide | nd | synthesis |
| LRSW-2 | LRSWIRRLIKAWKS-amide | nd | synthesis |
| LRSW-3 | LRSWRVIIRNGQR-amide | nd | synthesis |
| SelCPP2 | coum-DEEQERSEN-IRQIKIWFQNRRMKWK*K-amide | ++ | bEnd, Caco, |
| (FIG. 24) | | | SHS5Y |
| LYP-1 | CGNKRTRGC cyclic | − | Laakkonen |
| | | | 02 |
| Endo CPP | GWTLNSAGGKLKALAALAK | nd | synthesis |
| | Cys-s | | |
| | s-Cys-LLKKRKVVRLIKFLLK-amide | | |

*fluoresceinlabelled

MMP Activated selCPP

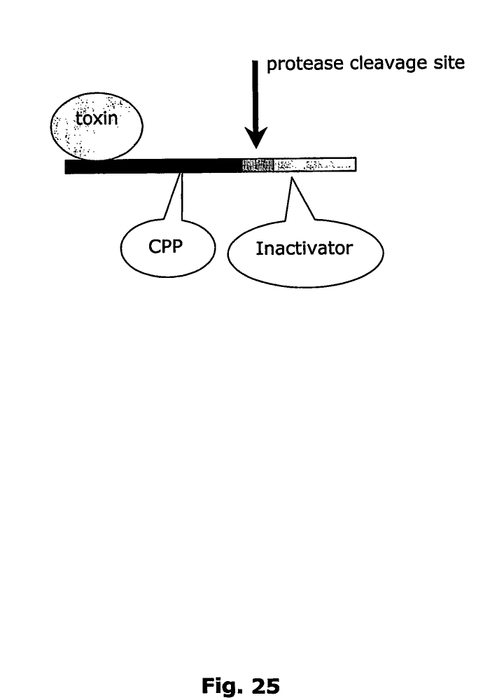

A typical selCPP (FIG. 25) is made up of three parts, the transporter (CPP), the specific protease site and the Inactivator. The Inactivator is added to Inactivate the CPP, so that it cannot enter cells before the inactivator is cleaved off. The CPP carries a toxin, for example a known non-permeable cytostatic and/or cytotoxic agent. For preferred embodiments of sequences and labelling of the peptides see table 13.

The Idea is based on three basic functions: a) selective cleavage (and thereby activation) by MMP-2 or MMP-MT1, b) cellular penetration by peptide (CPP) carrying the toxin and thereby c) killing of nearby, preferably tumour cells or endothelia involved in tumour neovascularisation (a schematic view is given in FIG. 25).

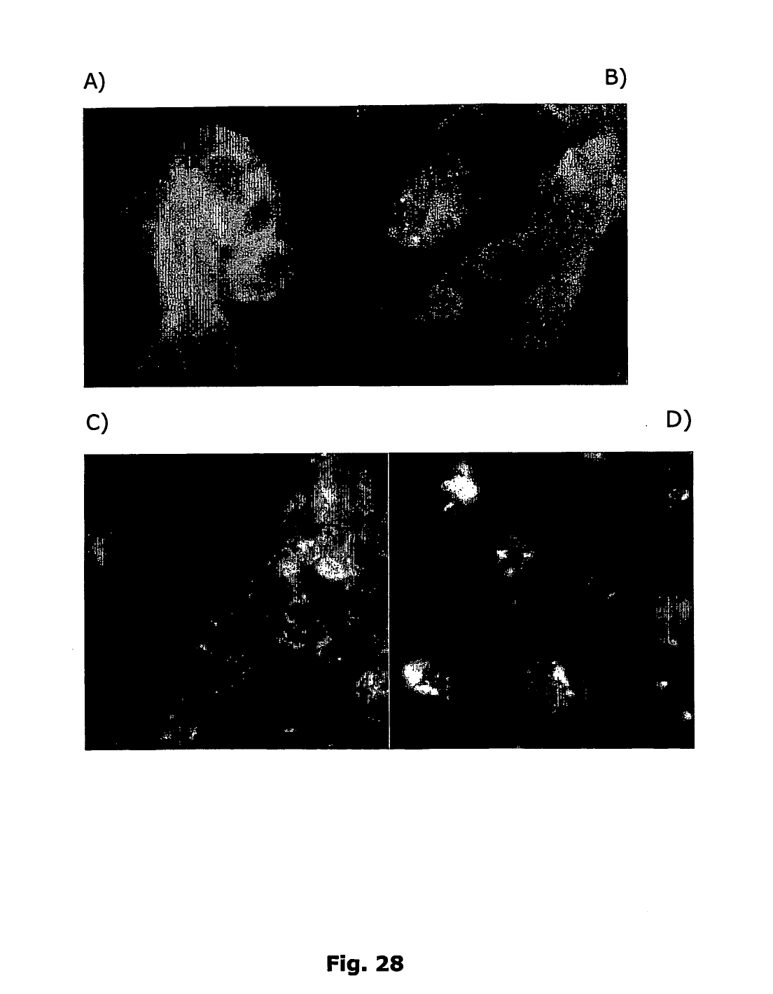

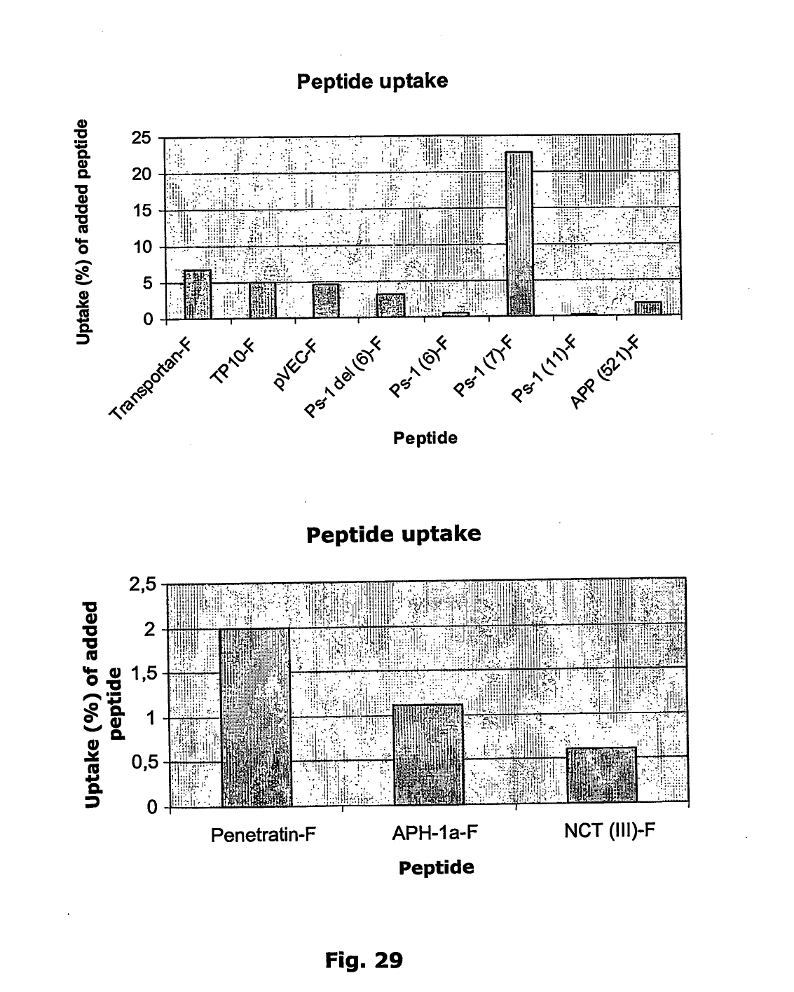

As an illustrative example to prove the above concept, the present inventors have successfully been able to show that the CPP-part of the selective CPP (YTA-2) can efficiently enter cells both at 370 (FIGS. 27 and 28) and 4° C. (data not shown). In addition, the “Inactivator”, see FIG. 26, renders the peptide less active in translocation over the cell membrane. The correct cleavage of YTA-2 by MMP2 has also be confirmed by mass spectrometry (data not shown). The attachment and efficiency of cytostatic and/or cytotoxic agent (MTX) to the CPP-part, see example 14.

There are four new sequences for cleavage of MMP 14 (or MMP-MT1): the LRSW1-3 and penMMP14. The latter has been shown to be taken up in cells expressing MMP14 (Bowes and Caco-2) see FIG. 24.

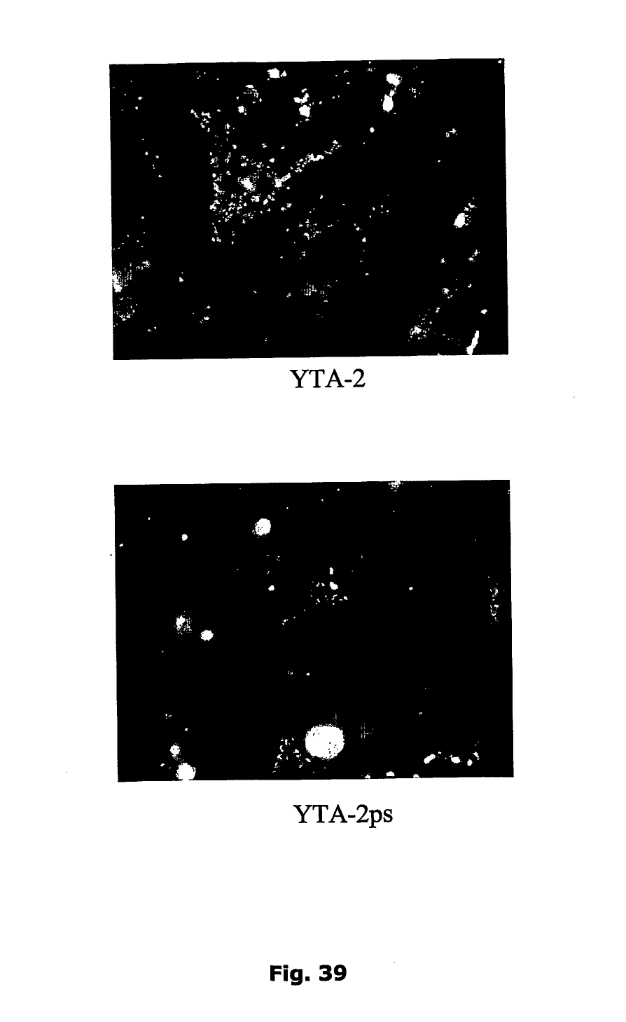

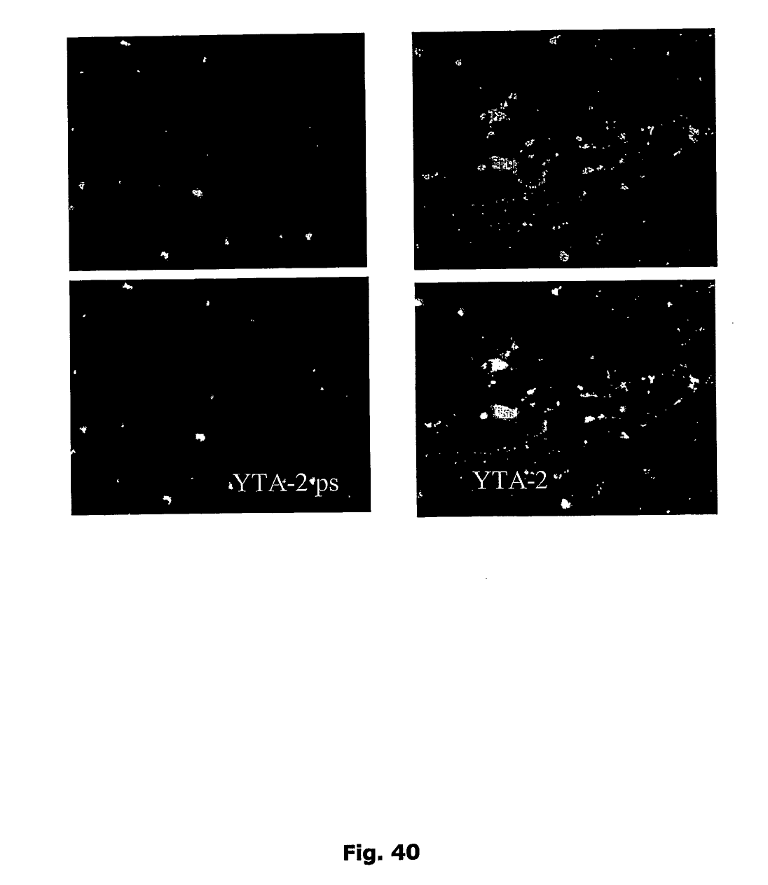

Additionally, the 67 kDa protein fluorescein labelled streptavidin was internalised when linked to blotinylated YTA-2 (see FIGS. 39 and 40). This shows that YTA-2 can transport large cargo molecules into cells.

TABLE 14
Epression of MMP2 and MMP14 in cell lines in the lab.

| | MMP2 expression | MMP14 expression |
|---|---|---|
| Cells: | (Experiment): | (Litterature) |
| Caco-2 | − | + |
| Lovo | − | + |
| SHS5Y | + (released inactive) | not determined |
| Bowes | + (released inactive) | + |
| PC 12 | + (released inactive) | not determined |
| Rinm5F | − | not determined |

All the selCPP tested for uptake so far are confirmed with mass spectrometry. Furthermore the stability of the peptides in cell culture media and in cell lysate are determined. In addition, the selectivity of the peptides are tested in in vitro cell assays, for example with cells expressing the selectively protease mixed with cells that do not express the protease for activation of the CPP part (Table 14.)

### Example 13

### Modification of Aβ-Production by Regulation of Secretase Activity

Amyloid Precursor Protein is processed in at least three different places by proteases called secretases. The secretases that are responsible for the creation of the Aβ-fragment, which is believed to be the main reason of toxicity in Alzheimer's Disease, are the β- and γ-secretases. Regulation of these secretases nowadays seems to be one of the most appealing approaches for developing a pharmaceutical aimed at reducing the Alzheimer's Disease symptoms.

Although this approach seems straightforward in inhibiting the AD symptoms, some caution is needed. This is mainly due to the fact that there is still some doubt as to whether the other fragments created in the secretase processing, sAPP (secretory APP) and AICD (APP intracellular domain), have some activity which is needed for the cells to work properly. Another problem to overcome is to be able to separate the Influence on APP from Notch, which in some ways seems to be processed in the same fashion as APP. In one embodiment of the present invention, an approach to this problem is taken comprising synthesising peptide fragments derived from the different secretases to find a peptide containing an ability to bind to a consensus sequence in the secretases/APP, thus competing with the secretase binding, which at the same time contains cell-penetrating ability. Said approach leads a transporter and deactivator in the same sequence, thus yielding a potential pharmaceutical against Alzheimer's Disease.

The sequences synthesised origin from Presenilin-1, nicastrin, APH-1 and PEN-2, which are important constituents in the γ-secretase complex. Also sequences from BACE, which is believed to be the β-secretase, is synthesised.

### Peptide Sequences.

TABLE 15
Peptides synthesised for affecting β-amyloid production.

| | |
|---|---|
| Ps-1-F 97-109 (6) del-I | VATKSVSVFYTRK deletion-I |
| Ps-1-F 97-109 (6) | VATIKSVSVFYTRK |
| Ps-1-F 151-162 (7) | VVLYKYRCYKVI |
| Ps-1-F 305-317 (11) | AQRRVSKNSKYNA |
| PS-1-F 151-165 | VVLYKYRCYKVIHAW |
| PS-1-F 151-165 H163R | VVLYKYRCYKVIRAW |
| PS-1-F 151-165 H163Y | VVLYKYRCYKVIYAW |
| PS-1-F 147-163 | TILLVVLYKYRCYKVIH |
| PS-1-F 147-163 H163R | TILLVVLYKYRCYKVIR |
| PS-1-F 148-163 H163R | ILLVVLYKYRCYKVIR |
| PS-1-F 149-163 H163R | LLVVLYKYRCYKVIR |
| PS-1-F 150-163 H163R | LVVLYKYRCYKVIR |
| PS-1-F 151-163 H163R | VVLYKYRCYKVIR |
| APP-F 521-537 (1) | KKAAQIRSQVMTHLRVI |
| APP-F 712-726 (3) | IATVIVITLVMLKKK |
| APP-F 712-726 V717F | IATVIVITLVMLKKK |
| APH-1a-F 97-109 | VFRFAYYKLLKKA |
| BACE-F 296-313 | RLPKKVFEAAVKSIKAAS |
| Nct-F 616-635 (I) | RLPRCVRSTARLARALSPAF |
| Nct-F 651-666 (II) | SRWKDIRARIFLIASK |
| Nct-F 414-434 (III) | RRPNQSQPLPPSSLQRFLRAR |
| PEN-2 11-26 | KLNLCRKYYLGGFAFL |
| PEN-2 63-75 | KGYVWRSAVGFLFW |
| PEN-2 78-89 | FQIYRPRWGALG |

### Methods

The γ-secretase cleavage is monitored through a luciferase reporter assay (Karlstrom et al. J Biol Chem 2002 Mar. 1; 277(9):6763-6)

The time points of peptide exposure were arbitrarily chosen since no previous data regarding these peptides could be consulted. The peptides were added twice, with 4 hours Incubation between the additions. After the incubation, the cells were washed with PBS, and lysed. After lysation, the luciferase activity and the protein concentrations was measured. The results are shown as % of control, were no peptide was added.

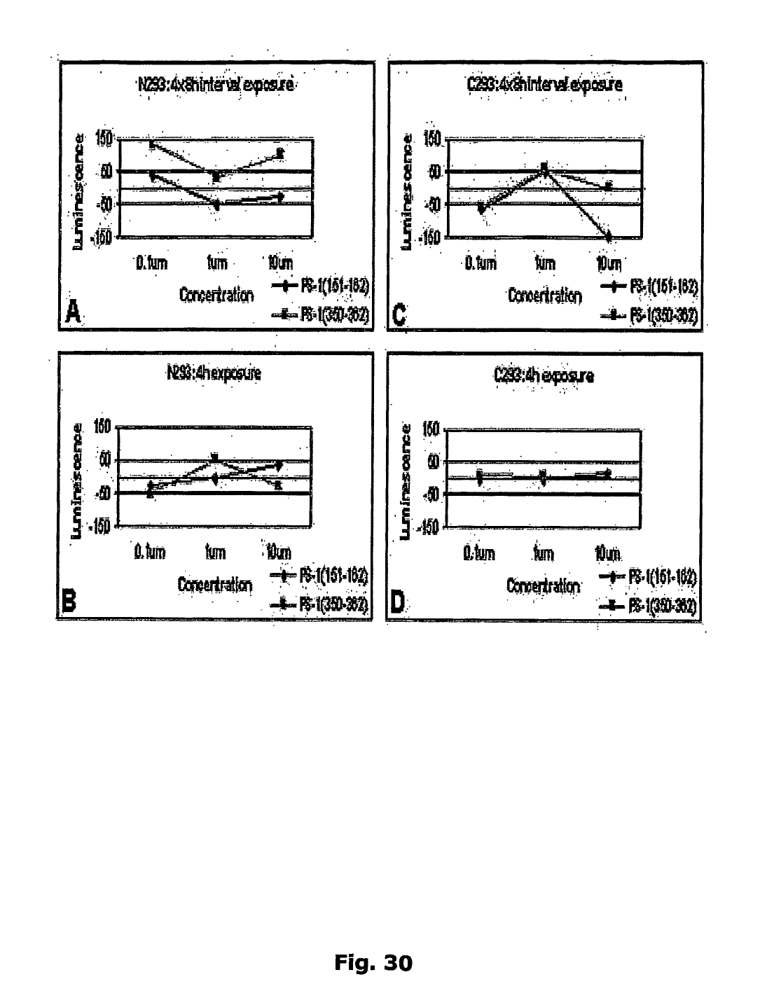

Luminescence rendered by presence of either peptide is shown in FIG. 30. A result above zero indicates an increase in APP cleavage compared to control, while data lower than zero represent the opposite. A difference can be seen in cleavage effects between the two procedures of peptide administration as well as between cell line responses. N293 is affected, of varying degree, regardless of time point, while the C293 cell line is primarily influenced by the longer exposure time. The two peptides seemingly follow one another in cleavage pattern but diverge in effect.

### Example 14

### Example of Intracellular Drug Delivery Conjugate of Methotrexate and Cell-Penetrating Peptide (MTX-CPP)

### Introduction

Methotrexate (MTX) is a cytotoxic drug, which was developed for the treatment of malignancies but is now also used to cure autoimmune diseases, such as psoriasis. MTX is a folate antagonist (Formule 1) and enters the cell via folate transporters. The targets of MTX are folate-requiring enzymes.

The usage of MTX in the treatment of psoriases is hampered due to its side-effects at the systemic administration, most serious is hepatotoxicity. Therefore, a topic administration formulation of MTX is highly desirable.

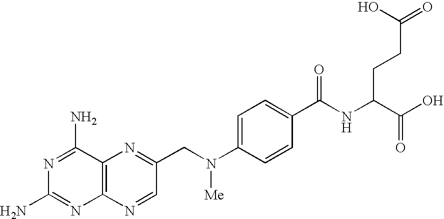

### Formule 1. The Structure of MTX.

In the biological fluids, MTX is present as negatively charged molecule. Therefore, it can cross the cell membrane only via folate transporters. The present invention for the first time reveals the means to produce an MTX-CPP conjugate which:
1) penetrates readily and receptor/carrier-independently into cells and into the skin
2) hampers the keratinocyte hyperproliferation, a hallmark of psoriasis
3) does not have unwanted side effects (such as histamine release).

All the MTX-CPP conjugates are synthesised using solid phase peptide synthesis strategy. This is possible due to two reasons:
1) MTX (CAS no 59-05-2) can be divided into two structural units: 4-deoxy-4-amino-N10-methylpteroic acid (Apa, CAS no 19741-14-1) and γ-glutamate
2) MTX is relatively insensitive to substitutions on its γ-carboxyl group

TABLE 16
MTX-CPP conjugates

Apa-γGlu-Gly-CPP
Apa-(γGlu)2-5-Gly-CPP
Apa-Cys-S-S-Cys-CPP

As shown in Table 16, several MTX-CPP conjugates are designed. It is expected that the CPP part of the conjugate is degraded in the cell.

### Solid Phase Synthesis of MTX-CPP Conjugates

Fmoc-chemistry was used due to MTX not being stable under standard cleavage conditions for Boc-chemistry (hydrogen fluoride at 0° C. for 30 min). The couplings of Fmoc-Glu-OtBu and Apa were performed using the standard coupling method (HOBt/TBTU). The conjugates were cleaved from the resing using the Reagent K (82.5% TFA: 5% phenol: 5% thioanisole: 2.5% 1,2-ethanedithiol).

### Cell Cultures

All cells were cultured at 37° C. in 5% CO₂. The plastic labware was from Corning Inc. (Acton, Mass.). Neonatal human epidermal keratinocytes (HEKn), all growth media components and trypsination reagents necessary for their propagation were obtained from Cascade Biologicsm (Portland, Oreg.). HEKn cells were cultured in Epilife® Medium supplemented with human keratinocyte growth supplement kit (HKGS) and penicillin, streptomycin and amphotericin B. HEKn cells were splitted once a week, seeding 300 000 cells per T75. Growth medium was changed every 1-2 days.

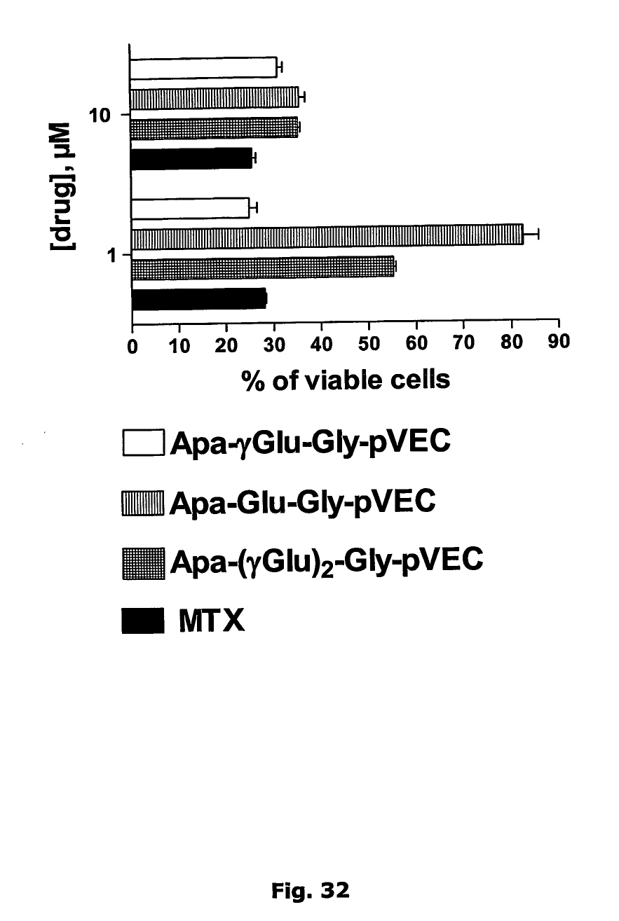

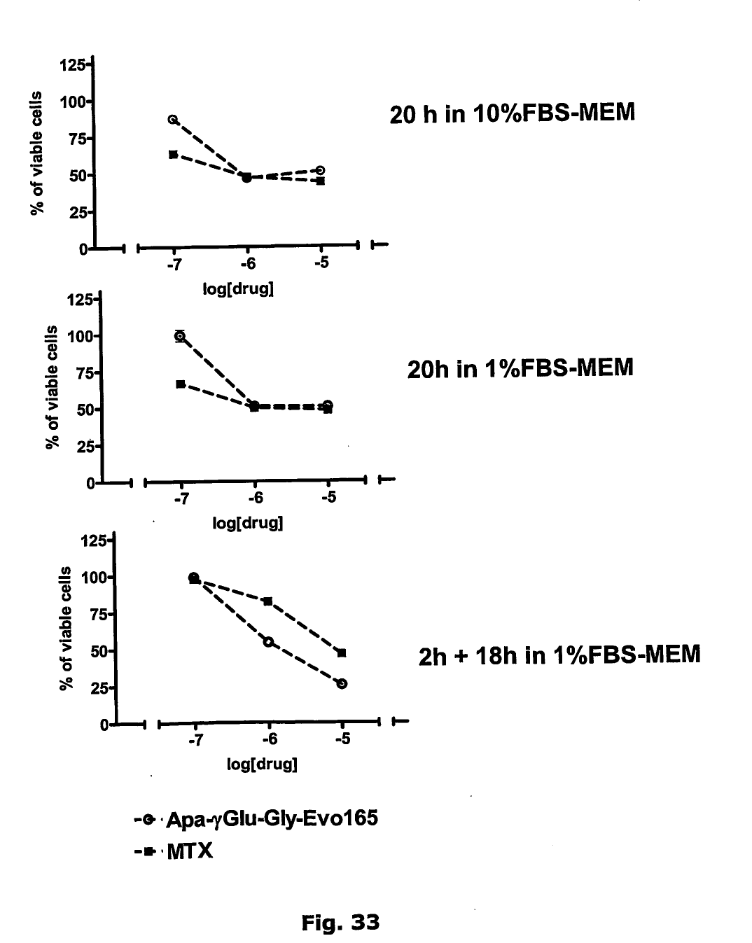

Bowes human melanoma cells were obtained from American Type Culture Collection (Manassas, Va.). Bowes cells were cultured in Minimal Essential Medium with Earie's sats complemented with Glutamax-I, non-essential amino acids, sodium pyruvate, penicillin, streptomycin (referred on figures as “MEM”) and 10% foetal bovine serum (FBS). All media components for Bowes cells were from invitrogen Corporation (Paisley, UK). Trypsin/EDTA was from PAA laboratories GmbH (Linz, Austria). Cells were subcultured once a week, seeding 200 000 cells per T75. For the viability assays, 50 000 cells (in 300 μl) were seeded per a well in a 24-well-plate a day before the experiment. K562 human erythroleukemia cells were obtained from American Type Culture Collection (Manassas, Va.). K562 cells were cultured in RPMI-1640 medium complemented with Glutamax-I, penicillin, streptomycin (referred in FIG. 32-35 as “RPMI”) and foetal bovine serum (7.5%). All media components for K562 cells were from invitrogen Corporation (Paisley, UK). Trypsin/EDTA was from PAA laboratories GmbH (Linz, Austria). Cells were subcultured every 2^{nd} or 3^{rd} day (when cell density had reached 1 000 000 cells/ml), seeding 100 000 cells per ml. For the viability assays, 30 000 cells in 300 μl were seeded per a well in a 24-well-plate.

### Cell Viability Measurements

The stock solutions of the conjugates (1 mM) were prepared in sterile water and the stock solution of MTX in 10% DMSO in water. For the exposure, the drugs were diluted in the exposure medium (serum-free OPTIMEM or 1% FBS in MEM or 10% FBS in MEM or 7.5% FBS in RPMI) to the desired concentrations (in the case of Bowes cells) or to 10× final concentration (in the case of K562 cells). 300 μl (for Bowes) or 30 μl (for K562) of the respective exposure mix was used per well. In the case of some experiments with Bowes cells, the exposure mix was removed after 2 h and replaced with prewarmed drug-free exposure medium. After 1-2 days the cell viability was assayed using CellTiter-Glo™ Luminescent Cell Viability Assay (Promega, Madison, Wis.). The plates were equilibrated to the room temperature (approximately 30 min). 300 μl of CellTiter-Glo™ Reagent was added to each well and incubated for 10 min. Then 300 μl was transferred to a white polystyrene FluoroNunc™ plate (Nunc A/S, Roskilde, Denmark) and luminescence was recorded at dual-scanning microplate spectrofluorometer (SPECTRAmax® GEMINI XS, Molecular Devices, Sunnyvale, Calif.).

### Results

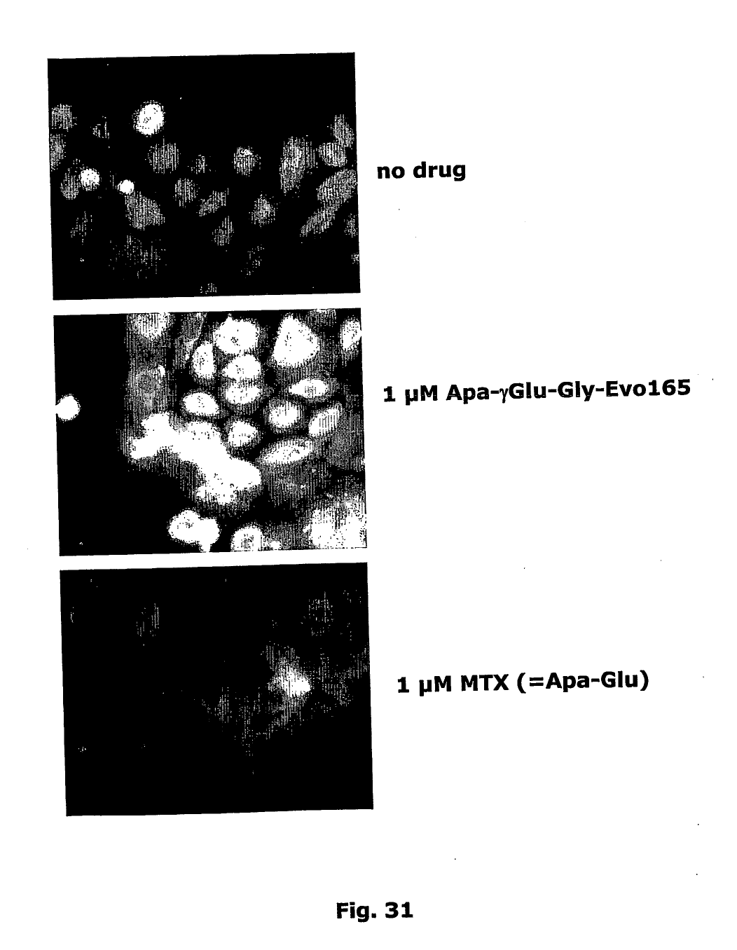

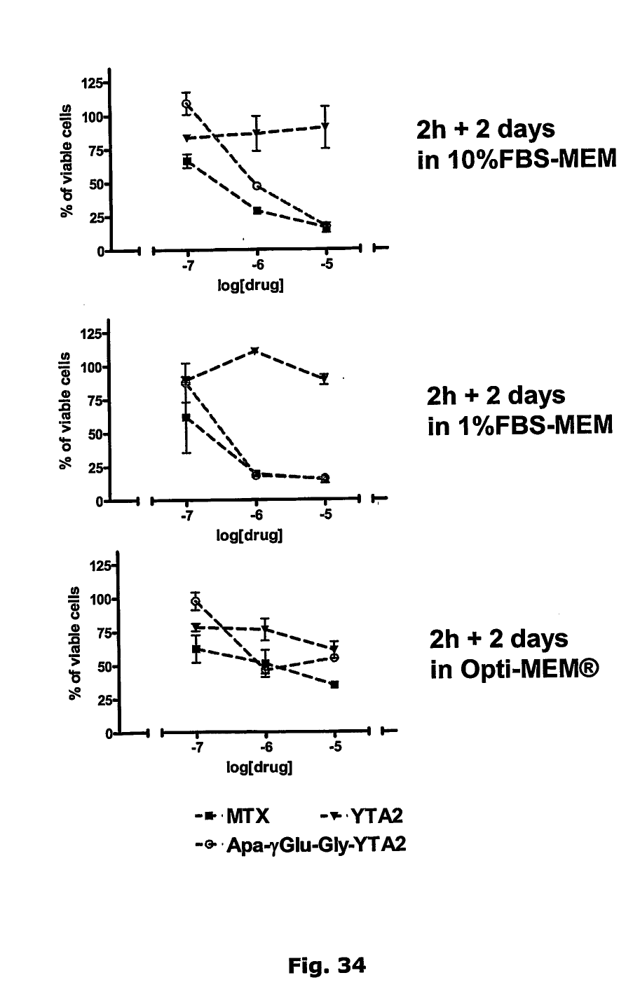

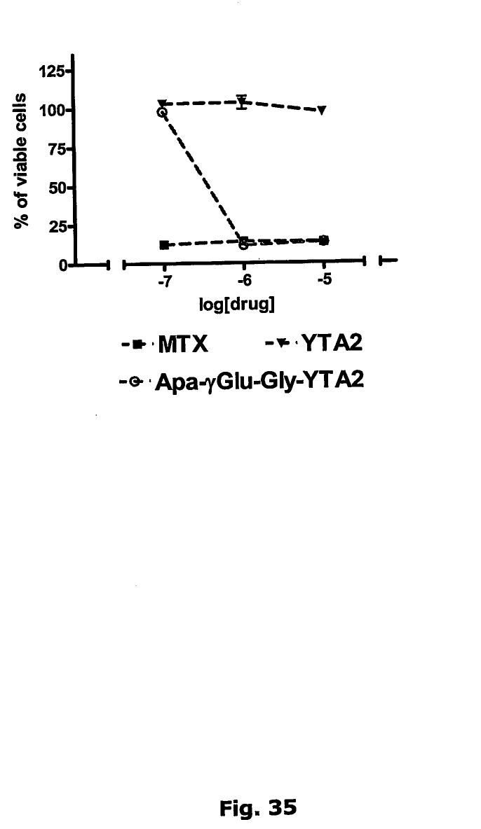

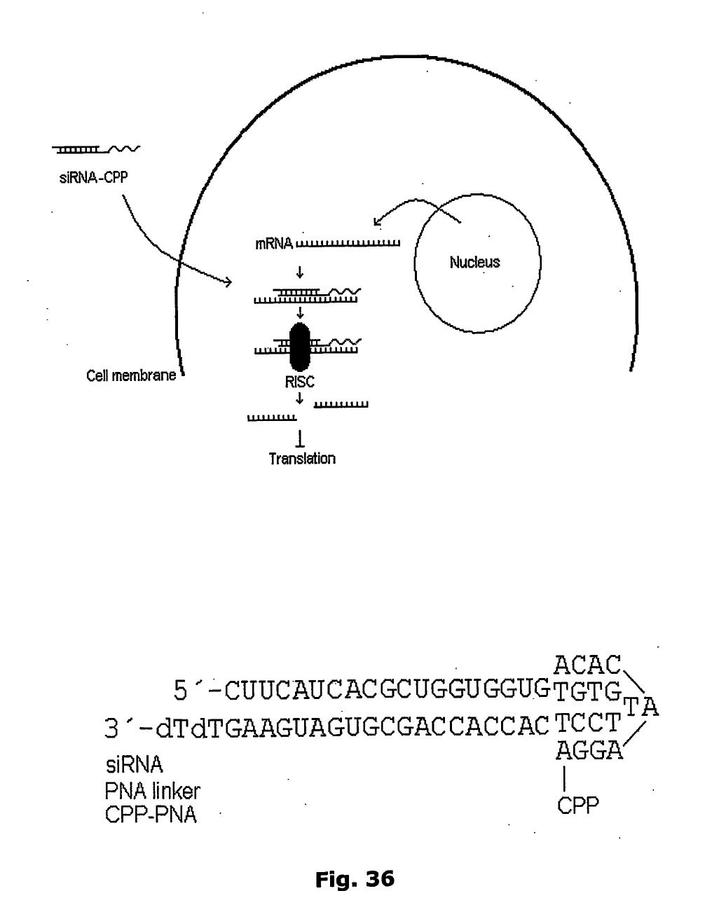

The results obtained so far indicate clearly that conjugation of an MTX to a CPP does not abolish the cell-penetrating nature of a CPP (as exemplified in FIG. 31, wherein Apa-γGlu-Gly-Evo165 is shown to penetrate into human epidermal keratinocytes). Also, the inventors find that the conjugation of a CPP to MTX does not abolish the toxic effect of MTX. Note that the γ-carboxyl group of MTX is more suitable for the conjugation to CPP than the α-carboxyl group of MTX (as seen in FIG. 32, wherein Apa-γGlu-Gly-pVEC is demonstrated to be more toxic than Apa-Glu-Gly-pVEC). What is more, a variety of different CPP-s can be used in the MTX-CPP conjugate as exemplified by all tested conjugates so far: Apa-γGlu-Gly-pVEC in FIG. 32, Apa-γGlu-Gly-Evo165 in FIG. 34 and Apa-γGlu-Gly-YTA2 in FIGS. 34 and 35).

### Example 15

### Improvement of siRNA Uptake using CPP

In resent years, small Interfering RNA (siRNA) have gained a lot of attention for their highly sensitive ability to regulate gene expression in mammalian cells. siRNA are short strands (about 20 bp) of double stranded RNA that Induce specific cleavage of their complementary mRNA through activation of the RNA-induced silencing complex (RISC). The RNA-induced silencing is an endogenous mechanism, but synthetically synthesized siRNA's have been shown to have the same effect both in vitro and In vivo.

Unfortunately, a typical problem when using siRNA is the low yield of uptake in the cell. By coupling cell-penetrating peptides (CPP) according to the present invention to synthetically synthesized siRNA the inventors were able to improve the cellular uptake both in vito and in vivo.

A siRNA was designed against the galanin receptor-1 (GALR-1) mRNA to which a CPP Transportan10 (Tp10) was coupled via a disulfide linker. The properties of the CPP were shown to increase the cellular uptake of the siRNA, thus rendering it more suitable of pharmaceutical usage. A schematic view of the mechanism of action is given in FIG. 39.

### Material and Methods:

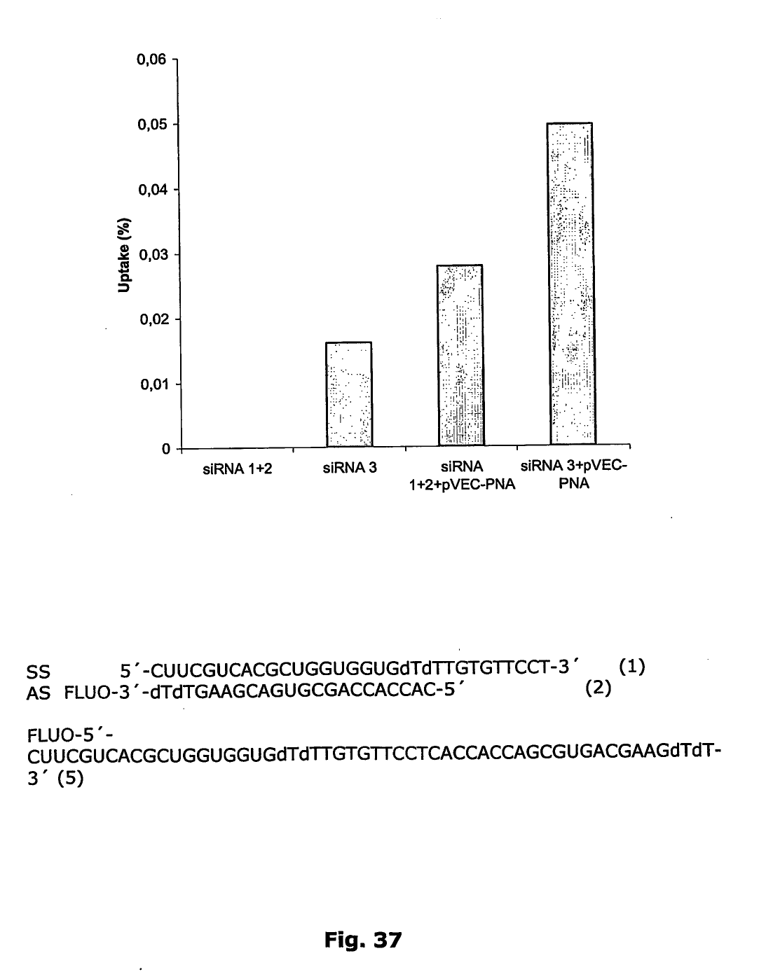

Bowes cells were grown over night in a 24 well plate (100 000 cells/well). The cells were treated with 200 μl, 1 μM fluorescently labelled peptide (pVEC), or fluorescently labelled siRNA-peptide (according to FIG. 37) for 30 min at 37° C. The cells were exposed to 3 times diluted standard trypsin/EDTA to remove any peptide stuck to the outer cell membrane. The cells were then lysed with 0.1% Triton-X and the uptake of peptide/siRNA was measured using Spectramax Gemeni XS fluorescence reader.

### Results:

As can be seen in FIG. 37, a clear difference was noted in uptake of CPP-conjugated DNA (siRNA) as compared to naked siRNA. The uptake was not due to membrane disruption by the cell-penetrating peptide. Furthermore mixture of un-conjugated pVEC and siRNA did not internalize into the cell.

As is well known in the field, siRNA has good efficiency even at low concentrations (down to pM) inside the cell, thus the uptake is considered enough to potentially activate the siRNA mediated degradation of the target mRNA.

### Example 16

### General Methods in Characterisation of Cell-Penetrating Peptides

### Transwell™ Experiments

The human colon cancer cell line Caco-2 (ATCC via LGC, Sweden) was propagated in Dulbeccos modified essential media with Glutamax (Invitrogen, Sweden) supplemented with 10% foetal bovine serum, sodium puruvate 1 mM, non-essential amino acids 1×100, 100 U/ml penicillin and 100 μg/ml streptomycinin air enriched with 5% CO₂ at 37° C.

Transwell™-clear cups (0.4 μm pores, Corning Costar, The Netherlands) were coated with bovine plasma fibronectin 0.5 μg/ml (Invitrogen, Sweden). 100 000 Caco-2 cells were seeded in each cup of a 12-well Transwell™ (1.13 cm2 filter area) and cultured for at least ten days. The media was changed in both the lower (1.5 ml) and the upper (0.5 ml) chambers every 2-3 days. The cell confluence was examined in a phase contrast microscope and by measuring TEER with a Millicell-ERS (Millipore, Sweden) with alternating current. As controls of the cell layer permeability, FITC-labelled dextran 4,4 kDa (Sigma-Aldrich, Sweden) passage was measured with or without 10 mM EGTA treatment. Before the addition of peptides, the media in the lower well was changed to HEPES buffered Krebbs-Ringer solution (HKR). The resistance reached 600 Ω/cm2 before the experiments were initiated, values over 500 Ω/cm2 are considered as high resistance.

Fluorophore-labelled peptide at 10 μM concentration, dissolved in phosphate buffered saline (PBS) was added to the upper Transwell™ chamber. At each time point, a 1501 sample was collected from the lower chamber, and the fluorescence was measured at 320/420 nm (Abz) and 492/520 nm (fluorescein) on a Spectramax Gemini XS (Molecular Devices, Calif.).

### Cellular Penetration Studies and Fluorescence Microscopy

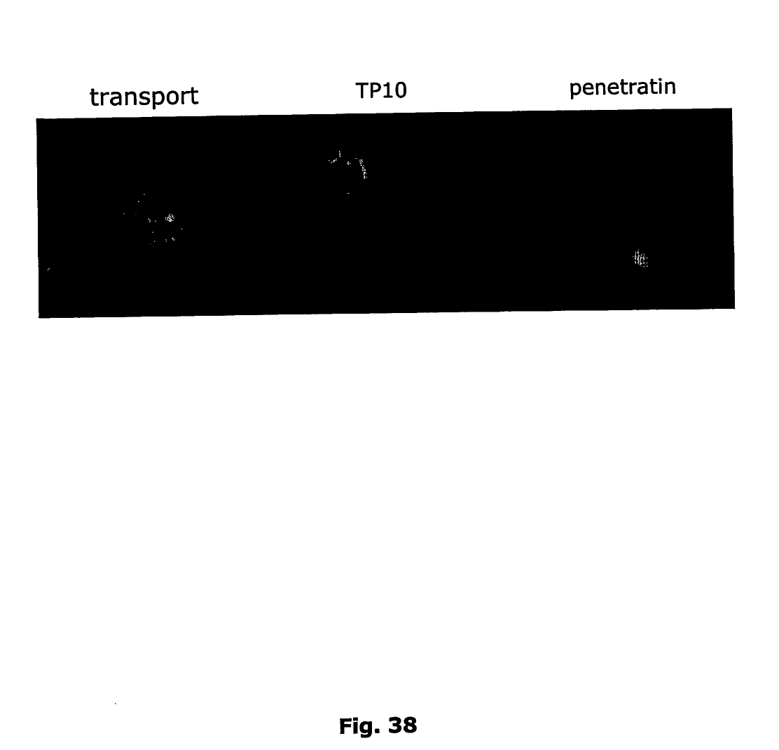

The cells were grown on round glass cover slips (12 mm, GTF, Sweden) in a 24-well plate to approximately 50% confluence. The media was changed to serum free and the biotinylated peptide solutions were added. The cells were incubated for 30 min at 37 or 4° C. The cells were washed twice with PBS, fixed with 4% paraformaldehyde solution 15 min at room temperature (dark) and then permeabilised in 30 mM HEPES buffer containing 0.5% w/v Triton X-100, 3 min on Ice. Sites for unspecific binding were blocked In PBS containing 3% (w/v) bovine serum albumin, overnight at 4° C. The peptides were visualised by staining with avidin-FrTC or streptavidin-TRITC (Molecular Probes, the Netherlands). The cell nuclei were stained with Hoechst 33258 (0.5 μg/ml) for 5 min, after which the cover slips were washed 3 times with PBS and mounted in 25% glycerol in PBS. The images were obtained with a Leica DM IRE2 fluorescence microscope (Leica Microsystem., Sweden) and processed in PhotoShop 6.0 software (Adobe Systems Inc., CA) (See e.g. FIG. 38-40).

### Peptide Uptake and Outflow Studies in Cells in Suspension

Cells were detached with trypsin (Invitrogen, Sweden), dissolved in culture media and centrifuged (1000×g for 10 min at RT). The cells were resuspended, counted and aliqoted in HKR on Ice, 300 000 cells/tube. Abz-labelled peptide was incubated for 15 and 30 min together with the cells in suspension, on a shaking 37° C. water bath. To stop the uptake or outflow, trypsin solution was added for 3 min. The cells were spun down at 1000×g for 10 min at 4° C. The pellets were resuspended in HKR for fluorescence detection, or for the outflow samples, incubated again with peptide-free HKR. Fluorescence was read at 320/420 nm on a Spectramax Gemini XS (Molecular Devices, Calif.). The Intracellular concentrations were calculated from a standard curve of Abz-labelled peptides. The average cell volume of Caco-2 cells was determined by using a Coulter 256 channelizer (Coulter Electronics Ltd. CA).

### Degradation of Peptides/Uptake in Cells Detected by Mass Spectrometry

Eighty percent confluent cells in 35-mm cell culture dishes were treated with 10 μM peptide dissolved in serum-free media for different time points. The cells were washed three times with PBS and cell lysates were prepared by treating the cells with 0.1% HCl for 15 min on Ice. The lysates were centrifuged at 13,000 g for 5 min and frozen. Before loading, the samples were purified by using C 18 Zip Tip columns (Millipore, Sweden) and then analysed on a Voyager-DR STR system (Applied Biosystems, Framingham).

### Membrane Disturbance Assays

### 2-deoxyglucose Assay

Cells were seeded in 12-well plates and used for experiment five days after seeding. First, 0.5 μCi of 2-deoxy-D-[1-3H]-glucose was added to each well (Amersham Pharmacia Biotech, UK) in glucose-free buffer. After 20 min incubation at 37° C. peptides were added in serum-free medium to reach the final concentrations of 5, 10 and 20 μM. As a positive control, cells were treated with 1% Triton X-100 in PBS (to establish the upper boundary of leakage). At 1, 5, 15 and 30 min a 150 μl media samples were collected and Emulsifier Safe scintillation cocktail (Packard, Netherlands) was added and the radioactivity was measured in a Packard 3255 liquid scintillation counter. The relative radioactive efflux from each well was calculated as percentage of untreated cells.

### Lactate Dehydrogenase Assay

Lactate dehydrogenase leakage was performed using CycioTox-ONEh Homeogeneous Membrane Integrity Assay from Promega Corp. (Promega, Madison, Wis.) and the lactate dehydrogenase activity calculated according to the manufacturer's Instructions.

### Histamine Release Assays

### Cell Culturing.

RBL-2H3 cells were obtained from American Type Culture Collection (Manassas, Va.). The cells were cultured in Minimal Essential Medium with Earle's salts complemented with Glutamax-I, non-essential amino acids, sodium pyruvate, penicillin, streptomycin and heat-inactivated foetal bovine serum (10%). All media components were from invitrogen Corporation (Paisley, UK). Trypsin/EDTA was from PAA laboratories Gmbh (Linz, Austria). Cells were splitted twice a week, seeding circa 1.2 million cells per T75. For the histamine release assay, 125 000-250 000 cells (in 1 ml) were seeded per a well in a 24-well-plate a day before the experiment.

### Histamine Release

The following solutions were used: assay buffer (10 mM HEPES, 140 mM NaCl, 5 mM KCl, 0.6 mM MgCl₂, 1 mM CaCl₂, 5.5 mM glucose, pH 7.4), 1 M NaOH, 10 mg/ml o-phthaldialdehyde (OPT) In methanol, 3 M HCl and 0.1% Triton X-100. All chemicals were from Sigma-Aldrich (St. Louis, Mass.). 1 mM peptide stock solutions were prepared in water and diluted further using assay buffer.

The cells were washed twice with assay buffer and then the peptide was added at desired concentration in 300 μl of assay buffer. For the determination of the total cellular histamine, some wells were exposed to 0.1% Triton X-100. After 20 min incubation at 37° C., the exposure medium was transferred to a 1.5 ml polypropylene tube and centrifuged briefly (2 min at 3000 rpm).

Histamine determination using OPT OPT was from Sigma-Aldrich Corp., St. Louis, Mo. 200 μl of the supernatant was transferred into a black untreated microwell plate (NUNC) and assayed for hivamine by adding 40 μl of 1 M NaOH and 10 μl of OPT solution and shaking for 4 minutes. To terminate the reaction, 20 μl of 3 M HCl was added. After 30 seconds, the fluorescence intensity was measured using a 355 nm excitation filter and a 455 nm emission filter (SPECTRAmax®GEMINI XS, Molecular Devices, Sunnyvale, Calif.). The histamine released was expressed as a percentage of total cellular histamine.

Histamine Determination using ELISA

The EUSA kit for the histamine was from IBL GmbH (Hamburg, Germany). The assay was performed following the manufacturers instructions. The absorbance measurements were performed using Digiscan Microplate Reader from ASYS Hitech GmbH (Eugendorf, Austria).

### [³⁵S]-GTPγS Binding Assay

The effect of the peptides on the initial rate of [³⁵S]-GTPS binding was determined by following the protocol presented by McKenzie with minor modifications. Briefly, the membranes were incubated with 50 000-70 000 cpm of [³⁵S]-GTPγS (Amersham Biosciences) In assay buffer (10 mM Tris-HCl, 0.1 mM EDTA, 5 mM MgCl2, 150 mM NaCl, 1 mM DTT, 10μM GDP, pH 7.5). The final protein concentration in the assay mixture, incubation time and temperature was adjusted to give the window in the linear part of the binding curve. Typically, the respective values were 1-2.5 mg/ml, 2-5 min and 15 or 25° C. The unbound [³⁵S]-GTPγS was removed by the addition of 0.9 ml of ice-cold TE buffer (10 mM Tris-HCl, 0.1 mM EDTA, pH 7.5) and rapid filtration of the reaction mixture through glass fiber filter without binder resin (Millipore, APFA 02500) which had been presoaked in at least 1 h in TE buffer. The filters were washed with 3×5 ml of ice-cold TE-buffer and transferred into counting vials. 10 ml of scintillation cocktail (Emulsifier-Safe, Packard) was added into each vial and the radioactivity counted next day.

### Example 17

### Effect of Intracellular Loop of CGRPR Loop (M630) on Porcine Coronary Artery

CGRP receptor loop iC4, sequence 391-405 (VQAILRRNWNQYKIQ) was synthesised and tested at blood vessels as described for the ATLAR loop previously.

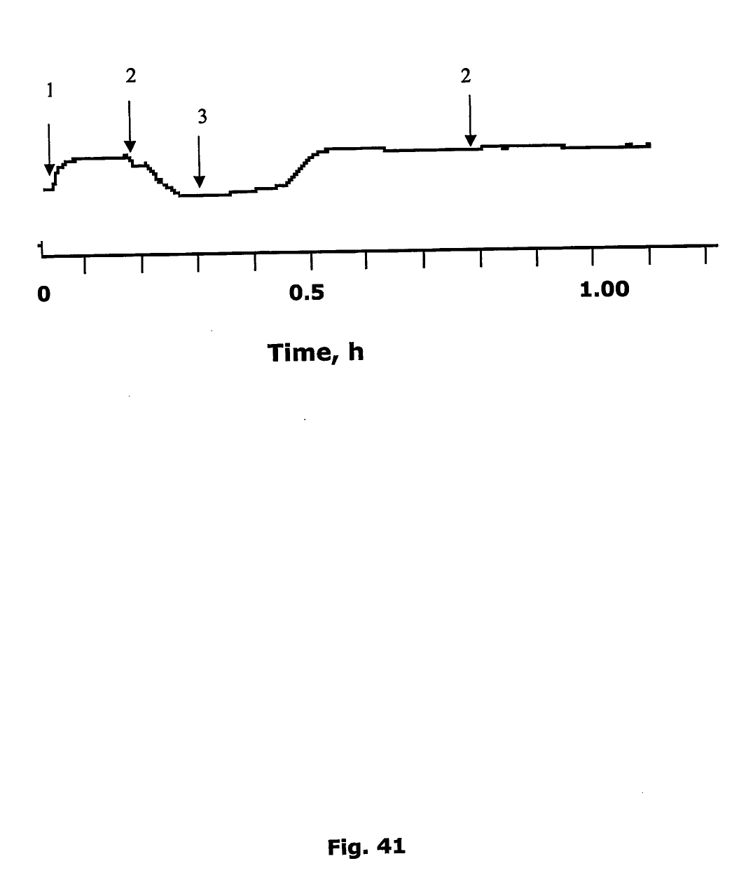

Porcine coronary artery was contracted by using KCl. After relaxation of the artery by washing, 50 μM M630 was applied. Contraction started after approximately 10 min and reached maximum in 15 to 20 min after application. Washing did not reverse the contraction. The effect was reproducible and always very clearly observed. The recording of typical experiment is shown in FIG. 41. The lag-period of 10 min could be the time needed for the penetration of M630 into the cells. M630 showed no effect on the contracted arteries.

Methods for this Experiment

### 1. Cell Cultures

Rin m5F cells were grown as monolayer culture in RPMI-1640 medium supplemented with 10% fetal bovine serum, 2 mM L-glutamine, 100 units/ml penicillin, 100 μml streptomycin at 37° C. in a 5% CO2 atmosphere.

Sf9 cells were maintained as monolayer culture in Grace's Insect medium supplemented with 10% heat-inactivated fetal bovine serum, 100 units/ml penicillin, 100 μg/ml streptomycin at 28° C. in a 5% CO₂ atmosphere.

### 2. Overexpression of G-Proteins in Sf9 Cells

Sf9 cells were cotransfected with recombinant Baculoviruses vectors carrying different alpha subunits of heterotrimeric G-proteins (Gαs, Gαi1, Gαo, Gα11) together with β1γ2 subunits as described by Nassman et al, with minor modifications. Briefly, approximately 6×10⁶ cells/75 cm2 flask were infected with high titer recombinant Baculovirus stock solution. After 60 min of incubation at 28° C. the virus stock was removed, the fresh medium was added and cells were grown for three days at 28° C. The expression of G-proteins was analyzed by 11% SDS-PAGE which, in contrast to control non-transfected cells, showed strong protein bands with molecular mass of 36 and 40-45 kDa which corresponded to β-subunits and α subunits of G-proteins, respectively. Each type of the overexpressed G-protein (Gs, Gl1, Go, G11) was further checked by using the corresponding monoclonal antibodies. The yield of the overexpression was assessed by comparison of the rate of [³⁵S]-GTPγS binding to the membranes obtained from transfected and non-transfected cells.

### 3. Plasma Membrane Preparation

Plasma membranes were obtained according to the protocol of McKenzie et al., 1992 with minor modifications described previously. Monolayer cell cultures were washed and then resuspended in TE buffer (10 mM Tris-HCl, 0.1 mM EDTA, pH 7.5). In the case of brain membranes, Wistar rats were first sacrificed and the whole brains were removed and sliced. The brain cortices were separated and quickly frozen in liquid nitrogen. Immediately before membrane preparation, tissue was chopped in small pieces. Material (tissue or cells) was homogenized in Polytron-type homogenizer (Braun AG, Germany). Homogenate was centrifuged at 500×g for 15 min and membranes were collected by centrifugation of the supernatant at 40 000×g for 30 min in Beckman-L8 70M ultracentrifuge (Beckman Instruments, USA). The whole procedure was undertaken at the temperature below 4° C. The protein concentration in membrane preparations was from 1 to 2.5 mg/ml as determined by the method of Lowry et al., 1952.

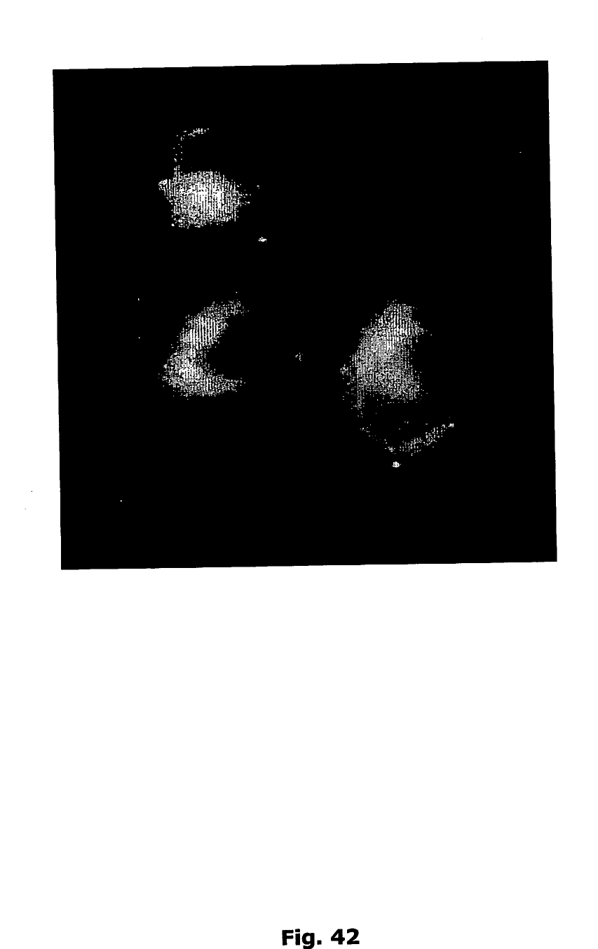

Cellular uptake of M630 was tested as described previously, the result is presented in FIG. 42.

### LIST OF REFERENCES

1. Lindgren, M., Hallbrink, M., Prochiantz, A. & Langel, Ü. Cell-penetrating peptides. Trends Pharmacol. Sci. 21, 99-103 (2000).
2. Derossi, D., Chassaing, G. & Prochiantz, A. Trojan peptides: the penetratin system for intracellular delivery. Trends Cell. Biol. 8, 8487 (1998).
3. Derossi, D., Joliot, A. H., Chassaing, G. & Prochiantz, A. The third helix of the Antennapedia homeodomain translocates through biological membranes. J. Blol. Chem. 269, 10444-10450 (1994).
4. Prochiantz, A. Homeodomain-derived peptides. In and out of the cells. Ann N Y Acad Sci 886, 172-9 (1999).
5. Sandberg, M., Eriksson, L., Jonsson, J., Sjöstrom, M. & Wold, S, New chemical descriptors relevant for the design of biologically active peptides. A multivariate characterization of 87 amino acids. J. Med. Chem. 41, 2481-2491 (1998).
6. Altschul, S. F., Gish, W., Miller, W., Myers, E. W. & Lipman, D. J. Basic local alignment search tool. J Mol Blol 215, 403-10. (1990).
7. Ma, H. & Diamond, S. L. Nonviral gene therapy and its delivery systems. Curr Pharm Biotechnol 2, 1-17. (2001).
8. Garnett, M. C. Gene-delivery systems using cationic polymers. Crit. Rev Ther Drug Carrier Syst 16, 147-207 (1999).
9. Somiarl, S. et al. Theory and in vivo application of electroporative gene delivery. Mol Ther 2, 178-87. (2000).
10. Lee, R.). & Huang, L. Uipidic vector systems for gene transfer. Crit. Rev. Therap. Drug Carrier Syst. 14, 173-206 (1997).
11. Ropert, C. Liposomes as a gene delivery system. Braz J Med Biol Res 32, 163-9. (1999).
12. Luo, D. & Saltzman, W. M. Enhancement of transfection by physical concentration of DNA at the cell surface. Nat Biotechnol 18, 893-5.
13. Feigner, P. L. et al. Nomenclature for synthetic gene delivery systems. Hum Gene Ther 8, 511-2. (1997).
14. Gebhart, C. L. & Kabanov, A. V. Evaluation of polyplexes as gene transfer agents. J Control Release 73, 401-16. (2001).
15. Boussif, O. et al. A versatile vector for gene and oligonucleotide transfer into cells In culture and In vivo: polyethylenimine. Proc Natl Acad Sci USA 92, 7297-301. (1995).
16. Abdallah, B. et al. A powerful nonviral vector for in vivo gene transfer into the adult mammalian brain: polyethylenimine. Hum Gene Ther 7, 1947-54. (1996).
17. Schatzlein, A. G. Non-viral vectors in cancer gene therapy: principles and progress. Anticancer Drugs 12, 275-304. (2001).
18. Remy-Kristensen, A., Clamme, J. P., Vuilleumier, C., Kuhry, J. G. & Mely, Y. Role of endocytosis in the transfection of L929 fibroblasts by polyethylenimine/DNA complexes. Biochim Biophys Acta 1514, 21-32. (2001).
19. Rozanov, D. V. et al. Mutation analysis of membrane type-1 matrix metalloproteinase (MT1-MMP). The role of the cytoplasmic tall Cys(574), the active site Glu(240), and furin cleavage motifs in oligomerization, processing, and self-proteolysis of MT1-MMP expressed in breast carcinoma cells. J Biol Chem 276, 25705-14. (2001)ⱼ.
20. Smith, L. E., Parks, K. K., Hasegawa, L. S., Eastmond, D. A. & Grosovsky, A. J. Targeted breakage of paracentromeric heterochromatin induces chromosomal Instability. Mutagenesis 13, 435-43. (1998).
21. Tang, W. et al. Development and evaluation of high throughput functional assay methods for HERG potassium channel. J Blol Screen 6, 325-31. (2001).
22. Hallbrink, M. et al. Cargo delivery kinetics of cell-penetrating peptides. Blochim Biophys Acta 1515, 101-9. (2001).
23. Vidal, P. et al. Interactions of primary amphipathic vector peptides with membranes. Conformational consequences and influence on cellular localization. J Membr Biol 162, 259-64. (1998).
24. Kilk, K. et al. Cellular Internalization of a cargo complex with a novel peptide derived from the third helix of the islet-1 homeodomain. Comparison with the penetratin peptide. Bioconjug Chem 12, 911-6. (2001).
25. Magzoub, M., Kilk, K., Eriksson, L. E., Langel, U. & Graslund, A. Interaction and structure induction of cell-penetrating peptides in the presence of phospholipid vesicles. Biochim Biophys Acta 1512, 77-89. (2001).
26. Inoue, A., Takahashi, M., Hatta, K., Hotta, Y. & Okamoto, H. Developmental regulation of islet-1 mRNA expression during neuronal differentiation in embryonic zebraflsh. Dev Dyn 199, 1-11. (1994).
27. Langel, U., Land, T. & Bartfal, T. Design of chimeric peptide ligands to galanin receptors and substance P receptors. Int J Pept Protein Res 39, 516-22. (1992).
28. McKenzie, F. R. Signal Transduction (Milligan, E., Ed.), Oxford University Press, Oxford, N.Y., Tokyo. (1992).
29. Lowry, O. H., Rosenbrough, N.Y., Farr, A. L. & Randall, R. J. J. Biol. Chem. 193, 265-275 (1951).
30. Cassel, D. & Selinger, Z. (1976) 452(2), 538-51. 452, 538-551 (1976).
31. Stemlicht M D, Werb Z., Annu Rev Cell Dev Biol 2001; 17:463-516 How matrix metalloproteinases regulate cell behavior.
32. Leppert D, Lindberg R L, Kappos L, Leib S L., Brain Res Brain Res Rev 2001 October; 36(2-3):249-57Matrix metalloproteinases: multifunctional effectors of Inflammation in multiple sclerosis and bacterial meningitis.
33. Rozanov D V, Deryugina E I, Ratnikov B I, Monosov E Z, Marchenko G N, Quigley J P, Strongin A Y., J Biol Chem 2001 Jul. 13; 276(28):25705-14, Mutation analysis of membrane type-1 matrix metalloproteinase (MT1-MMP). The role of the cytoplasmic tall Cys(574), the active site Glu(240), and furin cleavage motifs in oligomerization, processing, and self-proteolysis of MT1-MMP expressed in breast carcinoma cells.
34. Hoekstra R, Eskens F A, Verweij J, Oncologist 2001; 6(5):415-27, Matrix metalloproteinase Inhibitors: current developments and future perspectives.
35. Hornebeck W, Emonard H, Monboisse J C, Belion G., Semin Cancer Biol 2002 June; 12(3):231-41, Matrix-directed regulation of pericellular proteolysis and tumour progression.
36. Nabeshima K, Inoue T, Shimao Y, Sameshima T, Pathol Int 2002 April; 52(4):255-64 Matrix metalloproteinases in tumour invasion: role for cell migration.
37. Galvez B G, Matias-Roman S, Albar J P, Sanchez-Madrid F, Arroyo A G, J Biol Chem 2001 Oct. 5; 276(40):37491-500, Membrane type 1-matrix metalloproteinase is activated during migration of human endothelial cells and modulates endothelial motility and matrix remodeling.
38. Sounnl N E, Baramova E N, Munaut C, Maquoi E, Frankenne F, Foidart J M, Noel A., Int J Cancer 2002 Mar. 1; 98(1):23-8, Expression of membrane type 1 matrix metalloproteinase (MT1-MMP) In A2058 melanoma cells is associated with MMP-2 activation and increased tumour growth and vascularization.
39. Chen C C, Chen N, Lau L F., I Biol Chem 2001 Mar. 30; 276(13):10443-52, The angiogenic factors Cyr61 and connective tissue growth factor induce adhesive signaling in primary human skin fibroblasts.
40. Laakkonen P, Porkka K, Hoffman J A, Ruoslahti E, Nat Med 2002 July; 8(7):751-5, A tumour-homing peptide with a targeting specificity related to lymphatic vessels.
41. Pooga M., et al., 1998, FASEB 3. 1998 January; 12(1):67-77, Cell penetration by transportan.
42. Pooga M., et al., Nat. Biotechnol. 1998 September; 16(9):857-61, Cell-penetrating PNA constructs regulate galanin receptor levels and modify pain transmission in vivo.
43. Moroianu J., J. Cell Biochem, 1999, Suppl. 32-33:76-83, Nuclear import and export pathways.
44. Dayhoff, Schwartz, and Orcutt (1978) Atlas Protein Seq. Struc. 5:345-352 45. Henikoff and Henikoff (1992) Proc Natl Acad Sci USA 89(22):10915-9.
46. Simon R J et al., PNAS (1992) 89(20), 9367-9371 and Horwell D C, Trends Blotechnol. (1995) 13(4), 132-134.

1. Method for identifying a cell-penetrating peptide or protein and/or a cell-penetrating fragment of a peptide or protein, the method comprising the steps of
a) obtaining the amino acid sequence of said protein or peptide,
b) selecting the amino acid sequence of at least one candidate fragment,
d) assessing the bulk property value Z_{Σ} of said sequence, Z_{Σ} comprising at least 5 individual average interval values Z_{Σ1}; Z_{Σ2}; Z_{Σ3}; Z_{Σ4} and Z_{Σ5},
wherein Z_{Σ1}, Z_{Σ2}, Z_{Σ3}, Z_{Σ4} and Z_{Σ5} are average values of the respective descriptor values for the residues in said amino acid sequence, calculated with the formula
Z_{Σx}=(Zₓᵣₑₛ₁+Zₓᵣₑₛ₂ . . . +Zₓᵣₑₛₙ)/n
Z_{xresy} being the respective descriptor value for amino acid residue y comprised in the selected candidate fragment, and wherein the descriptor value of each residue corresponds to a Z₁, Z₂, Z₃, Z₄, and Z₅ descriptor value in a descriptor value scale as listed in table 1A, and
e) identifying a cell-penetrating fragment from said at least one candidate fragment(s) based on its Z_{Σ} bulk property value,
a cell-penetrating fragment being characterised by having a Z_{Σ} bulk property value essentially consisting of individual average interval values, wherein Z_{Σ1}<0.2; Z_{Σ2}<1.1; Z_{Σ3}<−0.49;
Z_{Σ4}<0.33; and Z_{Σ5}<1.1 and Z_{Σ5}>0.12,
f) optionally verifying the cell-penetrating capacity of said identified peptide or protein and/or said fragment by in vitro and/or in vivo methods.

2. Method for checking cellular penetration properties of a peptide, the method comprising the steps of
a) obtaining the amino acid sequence of the peptide,
d) assessing the bulk property value Z_{Σ} of said sequence, Z_{Σ} comprising at least 5 individual average interval values Z_{Σ1}: Z_{Σ2}; Z_{Σ3}; Z_{Σ4} and Z_{Σ5},
wherein Z_{Σ1}, Z_{Σ2}, Z_{Σ3}, Z_{Σ4} and Z_{Σ5} are average values of the respective descriptor values for the residues in said amino acid sequence, calculated with the formula
Z_{Σx}=(Zₓᵣₑₛ₁+Zₓᵣₑₛ₂ . . . +Zₓᵣₑₛₙ)/n
Z_{xresy} being the respective descriptor value for amino acid residue y comprised in the selected candidate fragment, and wherein the descriptor value of each residue corresponds to a Z₁, Z₂, Z₃, Z₄, and Z₅ descriptor value in a descriptor value scale as listed in table 1A, and
e) checking the cell-penetrating properties of said peptide based on its Z_{Σ} bulk property value,
a cell-penetrating fragment being characterised by having a Z_{Σ} bulk property value essentially consisting of individual average interval values, wherein Z_{Σ1}<0.2; Z_{Σ2}<1.1; Z_{Σ3}<−0.49; Z_{Σ4}<0.33; and Z_{Σ5}<1.1 and Z_{Σ5}>0.12,
f) synthesizing or isolating a peptide comprising the amino acid sequence of said identified cell-penetrating peptide, and
g) optionally verifying the protein-mimicking functionality and/or the cell-penetrating capacity of the synthesized or isolated peptide by in vitro and/or in vivo methods.

3. Method for producing a cell-penetrating and functional protein-mimicking peptide, the method comprising the steps of
a) selecting a functional protein of interest,
b) obtaining the amino acid sequence of said selected protein,
c) selecting the amino acid sequence of at least one candidate fragment corresponding to an intracellular part of said protein,
d) assessing the bulk property value Z_{Σ} of said sequence, Z_{Σ} comprising at least 5 individual average interval values Z_{Σ1}; Z_{Σ2}; Z_{Σ3}; Z_{Σ4} and Z_{Σ5},
wherein Z_{Σ1}, Z_{Σ2}, Z_{Σ3}, Z_{Σ4} and Zs are average values of the respective descriptor values for the residues in said amino acid sequence, calculated with the formula
Z_{Σx}=((Zₓᵣₑₛ₁+Zₓᵣₑₛ₂ . . . +Zₓᵣₑₛₙ)/n
Z_{xresy} being the respective descriptor value for amino acid residue y comprised in the selected candidate fragment, and wherein the descriptor value of each residue corresponds to a Z₁, Z₂, Z₃, Z₄, and Z₅ descriptor value in a descriptor value scale as listed in table 1A, and
e) identifying a cell-penetrating fragment from said at least one candidate fragment(s) based on its Z_{Σ} bulk property value,
a cell-penetrating fragment being characterised by having a Z_{Σ} bulk property value essentially consisting of individual average interval values, wherein Z_{Σ1}<0.2; Z_{Σ2}<1.1; Z_{Σ3}<−0.49; Z_{Σ4}<0.33; and Z_{Σ5}<1.1 and Z_{Σ5}>0.12,
f) synthesizing or isolating a peptide comprising the amino acid sequence of said identified cell-penetrating peptide, and
g) optionally verifying the protein-mimicking functionality and/or the cell-penetrating capacity of the synthesized or isolated peptide by in vitro and/or in vivo methods.

4. Method for de novo designing and producing an artificial cell-penetrating and/or an artificial cell-penetrating and functional protein-mimicking peptide, the method comprising the steps of
a) designing at least one artificial peptide and/or peptide fragment,
d) assessing the bulk property value Z_of the amino acid sequence of said artificial peptide or peptide fragment, Z_comprising at least 5 individual average interval values Z_{Σ1}; Z_{Σ2}; Z_{Σ3}; Z_{Σ4} and Z_{Σ5},
wherein Z_{Σ1}, Z_{Σ2}, Z_{Σ3}, Z_{Σ4} and Z_{Σ5} are average values of the respective descriptor values for the residues in said amino acid sequence, calculated with the formula
Z_{Σx}=(Zₓᵣₑₛ₁+Zₓᵣₑₛ₂ . . . +Zₓᵣₑₛₙ)/n
Z_{xresy} being the respective descriptor value for amino acid residue y comprised in the selected candidate fragment, and wherein the descriptor value of each residue corresponds to a Z₁, Z₂, Z₃, Z₄, and Z₅ descriptor value in a descriptor value scale as listed in table 1A, and
e) checking the cell-penetrating properties of said artificial peptide and/or peptide fragment based on its Z_{Σ} bulk property value,
a cell-penetrating fragment being characterised by having a Z_{Σ} bulk property value essentially consisting of individual average interval values, wherein Z_{Σ1}<0.2; Z_{Σ2}<1.1; Z_{Σ3}<−0.49; Z_{Σ4}<0.33; and Z_{Σ5}<1.1 and Z_{Σ5}>0.12,
f) synthesizing said peptide and/or peptide fragment comprising the amino acid sequence identified as cell penetrating, and
g) optionally verifying the protein-mimicking functionality and/or the cell-penetrating capacity of the synthesized peptide and/or peptide fragment by in vitro and/or in vivo methods.

5. Method according to any of claims 1-4, wherein said amino acid sequence after step e) is additionally
h) assessed and selected for having a property value essentially consisting of individual average interval values, wherein Z_{ΣBukha}>3.1 and Z_{ΣBulkha}<8.13 and Z_{Σ1}>−1.25 and Z_{Σ1}<3.52 and Z_{Σ2}>−3.9 and Z_{Σ2}<3.1 and Z_{Σ3}<−0.5 and Z_{Σ3}>−3.51 and Z_{Σhdb}>−0.1 5 and Z_{Σhdb}<5.1 and hdb>0 and hdb<84.

6. Method according to any of claims 1-4, wherein said amino acid sequence after step e) is additionally
h) assessed and selected for having a property value essentially consisting of individual average interval values, wherein Z_{ΣBulkha}>3.2 and Z_{ΣBulkha}<5.9 and Z_{Σ1}>−1.25 and Z_{Σ1}<1.92 and Z_{Σ2}>−1.22 and Z_{Σ2}<1.29 and Z_{Σ3}<−0.5 and Z_{Σ3}>−1.94 and Z_{Σhdb}>0.28 and Z_{Σhdb}<2 and hdb>5 and hdb<30.

7. Method according to any of claims 1-4, wherein said amino acid sequence after step e) is additionally
h) assessed and selected for having a property value essentially consisting of individual average interval values, wherein Z_{ΣBulkha}>3.2 and Z_{ΣBulkha}<4.8 and Z_{Σ1}>−1.1 and Z_{Σ1}<1.92 and Z_{Σ2}>−1.1 and Z_{Σ2}<0 and Z_{Σ3}<−0.55 and Z_{Σ3}>−1.94 and Z_{Σhdb}>−0.28 and Z_{Σhdb}<1.57 and hdb>7 and hdb<25.

8. Method according to any of claims 1-4, wherein steps d) and e) are exchanged for
h) assessing and selecting an amino acid sequence for having a property value essentially consisting of individual average interval values, wherein Z_{ΣBulkha}>3.1 and Z_{ΣBulkha}<8.13 and Z_{Σ1}>−1.25 and Z_{Σ1}<3.52 and Z_{Σ2}>−3.9 and Z_{Σ2}<3.1 and Z_{Σ3}<−0.5 and Z_{Σ3}>−3.51 and Z_{Σhdb}>−0.115 and Z_{Σhdb}<5.1 and hdb>0 and hdb<84.

9. Method according to any of claims 1-4, wherein steps d) and e) are exchanged for
h) assessing and selecting an amino acid sequence for having a property value essentially consisting of individual average interval values, wherein Z_{ΣBulkha}>3.2 and Z_{ΣBulkha}<5.9 and Z_{Σ1}>−1.25 and Z_{Σ1}<1.92 and Z_{Σ2}>−1.22 and Z_{Σ2}<1.29 and Z_{Σ3}<−0.5 and Z_{Σ3}>−1.94 and Z_{Σhdb}>0.28 and Z_{Σhdh}<2 and hdb>5 and hdb<30.

10. Method according to any of claims 1-4, wherein steps d) and e) are exchanged for
h) assessing and selecting an amino acid sequence for having a property value essentially consisting of individual average interval values, wherein Z_{ΣBulkha}>3.2 and Z_{ΣBulkha}<4.8 and Z_{Σ1}>−1.1 and Z_{Σ1}<1.92 and Z_{Σ2}>−1.1 and Z_{Σ2}<0 and Z_{Σ3}<−0.55 and Z_{Σ3}>−1.94 and Z_{Σhdb}>−0.28 and Z_{Σhdb}<1.57 and hdb>7 and hdb<25.

11. A method according to any of claims 1 to 4, wherein said protein is a transmembranal protein.

12. A method according to claim 11, wherein said protein is a protein selected from the group consisting of human PrpC, bovine PrpC, amyloid precursor protein (APP) and presenilin-1 (PS-1).

13. A method according to claim 11, wherein said protein is a mammalian receptor, such as a receptor belonging to the superfamily of tyrosine kinase receptors, a 7™ recptor and/or a G-protein coupled receptor.

14. A method according to claim 13, wherein said protein is a protein selected from the group consisting of the GLP-1 receptor, AT1A receptor, and Dopamine-2 receptor.

15. A method according to any of claims 1 to 4, wherein the cell-penetrating capacity of said peptide and/or peptide fragment is verified by monitoring the cellular uptake rate of a detectable dye into said cell after exposure to said peptide and/or peptide fragment.

16. A method according to claim 15, wherein said dye is fluorescein.

17. A cell-penetrating peptide and/or a non-peptide analogue thereof obtained by a method according to any of claims 1 to 4.

18. A cell-penetrating peptide essentially consisting of a peptide obtained by a method according to any of claims 1 to 4.

19. A cell-penetrating peptide selected from a 8 to 50 amino acid residues long peptide, or a fragment thereof with cell-penetrating capacity.

20. A cell-penetrating peptide according to claim 19, wherein the peptide is 14 to 30 amino acid residues long.

21. A cell-penetrating peptide according to claim 19, wherein the peptide is 16 to 20 amino acid residues long.

22. A cell-penetrating peptide selected from a 8 amino acid residues long peptide or a fragment of a peptide corresponding to one of the amino acid sequences listed in SEQ.ID.NO. 6234-7420.

23. A cell-penetrating peptide selected from a 12 to 50 amino acid residues long peptide or a fragment of a peptide corresponding to one of the amino acid sequences listed in SEQ.ID.NO. 1-150.

24. A cell-penetrating peptide selected from a 12 amino acid residues long peptide or a fragment of a peptide corresponding to one of the amino acid sequences listed in SEQ.ID.NO. 151-2684.

25. A cell-penetrating peptide selected from a 12 amino acid residues long peptide or a fragment of a peptide corresponding to one of the amino acid sequences listed in SEQ.ID.NO. 7421-11649.

26. A cell-penetrating peptide selected from a 16 amino acid residues long peptide or a fragment of a peptide corresponding to one of the amino acid sequences listed in SEQ.ID.NO. 2685-6233.

27. A cell-penetrating peptide selected from a 16 amino acid residues long peptide or a fragment of a peptide corresponding to one of the amino acid sequences listed in SEQ.ID.NO. 11650-18398.

28. A cell-penetrating functional protein-mimicking peptide that is derived from a transcription factor or designed to closely resemble a transcription factor or at least a functional fragment of a transcription factor.

29. A cell-penetrating peptide selected from a 8-16 amino acid residues long peptide or a fragment of a peptide corresponding to one of the amino acid sequences listed in SEQ.ID.NO. 18399-31839.

30. A cell-penetrating functional protein-mimicking peptide that is derived from a secretase or designed to closely resemble a secretase or at least a functional fragment of a secretease.

31. A cell-penetrating peptide selected from a peptide or a fragment of a peptide corresponding to one of the amino acid sequences listed in SEQ.ID.NO. 31840-31864.

32. A cell-penetrating functional protein-mimicking peptide that is derived from a GLP-1 receptor or designed to closely resemble a GLP-1 receptor or at least a functional fragment of a GLP-1 receptor.

33. A cell-penetrating peptide selected from a peptide or a fragment of a peptide corresponding to one of the amino acid sequences listed in SEQ.ID.NO. 31865-31886.

34. A cell-penetrating functional protein-mimicking peptide that is derived from a CGRP receptor or designed to closely resemble a CGRP receptor or at least a functional fragment of a CGRP receptor.

35. A cell-penetrating peptide selected from a peptide or a fragment of a peptide corresponding to the amino acid sequence listed in SEQ.ID.NO. 31895.

36. A cell-penetrating functional protein-mimicking peptide that is derived from an AT2 type receptor or designed to closely resemble an AT2 type receptor or at least a functional fragment of an AT2 type receptor.

37. A cell-penetrating peptide selected from a peptide or a fragment of a peptide corresponding to one of the amino acid sequences listed in SEQ.ID.NO. 31887-31894.

38. A cell-penetrating functional protein-mimicking peptide that is derived from a PrpC or designed to closely resemble a PrpC or at least a functional fragment of a PrpC.

39. A cell-penetrating peptide selected from a peptide or a fragment of a peptide corresponding to one of the amino acid sequences listed in SEQ.ID.NO. 31896-31899.

40. A cell-penetrating functional protein-mimicking peptide that is derived from amyloid precursor protein (APP) or presenilin-1 (PS-1) or designed to closely resemble amyloid precursor protein (APP) or presenilin-1 (PS-1) or at least a functional fragment of amyloid precursor protein (APP) or presenilin-1 (PS-1).

41. A cell-penetrating peptide selected from a peptide or a fragment of a peptide corresponding to one of the amino acid sequences listed in SEQ.ID.NO. 31900-31906.

42. A functional analogue of a cell-penetrating peptide according to any of claims 19 to 41.

43. A cell-penetrating peptide and/or a non-peptide analogue thereof being at least 75% identical to a cell-penetrating peptide and/or a non-peptide-analogue thereof according to any of claims 19 to 41.

44. A cell-penetrating peptide and/or a non-peptide analogue thereof comprising a cell-penetrating peptide and/or a non-peptide analogue thereof according to any of claims 19 to 41.

45. A cell-penetrating peptide and/or a non-peptide analogue thereof according to any of claims 19 to 41, selected from the group consisting of peptides comprising the amino acid sequence IVIAKLKA and/or a cell membrane penetrating functional analogue thereof.

46. A cell-penetrating peptide and/or a non-peptide analogue thereof according to claim 45, comprising the amino acid sequence IVIAKLKANLMCKTCRLAK.

47. A cell-penetrating peptide and/or a non-peptide analogue thereof according to any of claims 19 to 41, wherein the peptide is coupled to a cargo.

48. A cell-penetrating peptide and/or a non-peptide analogue thereof according to claim 47, wherein the peptide is coupled to a cargo by a S—S bridge.

49. A cell-penetrating peptide and/or a non-peptide analogue thereof according to claim 47, wherein the cargo is a cellular effector.

50. A cell-penetrating peptide and/or a non-peptide analogue thereof according to claim 47, wherein the cargo is a pharmaceutically active component.

51. A cell-penetrating peptide and/or a non-peptide analogue thereof according to claim 47, wherein the cargo is selected from the group consisting of a small molecule, peptide, protein, saccharide, single and/or double stranded oligonucleotide, plasmid, antibiotic substance, cytotoxic and/or antiviral agent.

52. A cell-penetrating peptide and/or a non-peptide analogue thereof according to claim 47, wherein the cargo is a marker molecule.

53. A cell-penetrating peptide and/or a non-peptide analogue thereof according to claim 47, selected from a peptide or a fragment of a peptide corresponding to one of the amino acid sequences listed in SEQ.ID.NO. 31907-31922.

54. A cell-selective delivery system for a cytostatic and/or cytotoxic agent, comprising
a) a cell-penetrating peptide and/or a non-peptide analogue thereof comprising a protease consensus site for a protease specifically overexpressed in and/or secreted by a target cell and
b) a cytostatic and/or cytotoxic agent, wherein said cell-selective delivery system additionally comprises an inactivation sequence repressing the activity of said cell-penetrating peptide, and which is cleaved by said protease upon introducing said cell-selective delivery system in the near vicinity of said target cell.

55. A vector for transfecting a cell, the vector comprising
a) a nucleic acid component,
b) a polycation conjugate, and
c) a cell-penetrating peptide and/or a non-peptide analogue thereof, wherein the average rate of transfection per cell at identical transfection conditions is enhanced by a factor of at least 2, compared to a vector comprising only components a) and b), or only components a) and c).

56. A vector according to claim 55, wherein said vector is used in a transient transfection and/or a stable transfection of a cell.

57. A vector according to claim 56, wherein said vector is used in an in vivo and/or in an in vitro transfection of a cell.

58. A vector according to claim 57, wherein said vector is used for a non-viral transfection of a cell.

59. A vector according to any of claims 55-58, wherein said polycation conjugate is polyethylene imine (PEI).

60. A vector according to claim 55, selected from a peptide or a fragment of a peptide corresponding to the amino acid sequence listed in SEQ.ID.NO. 31913.

61. A vector according to any of claims 55-58, wherein said cell-penetrating peptide is a peptide or a peptide fragment is selected from the group consisting of an 8 amino acid residues long peptide or a fragment of a peptide corresponding to one of the amino acid sequences listed in SEQ ID NOS:6234-7420, a 12 to 50 amino acid residues long peptide or a fragment of a peptide corresponding to one of the amino acid sequences listed in SEQ ID NOS:1-150, a 12 amino acid residues long peptide or a fragment of a peptide corresponding to one of the amino acid sequences listed in SEQ ID NOS:151-2684, a 12 amino acid residues long peptide or a fragment of a peptide corresponding to one of the amino acid sequences listed in SEQ ID NOS:7421-11649, a 16 amino acid residues long peptide or a fragment of a peptide corresponding to one of the amino acid sequences listed in SEQ ID NOS:2685-6233, a 16 amino acid residues long peptide or a fragment of a peptide corresponding to one of the amino acid sequences listed in SEQ ID NOS: 11650-18398, an 8-16 amino acid residues long peptide or a fragment of a peptide corresponding to one of the amino acid sequences listed in SEQ ID NOS: 18399-31839, a peptide or a fragment of a peptide corresponding to one of the amino acid sequences listed in SEQ ID NOS:31840-31864, a peptide or a fragment of a peptide corresponding to one of the amino acid sequences listed in SEQ ID NOS:31865-31886, a peptide or a fragment of a peptide corresponding to the amino acid sequence of SEQ ID NO:31895, a peptide or a fragment of a peptide corresponding to one of the amino acid sequences listed in SEQ ID NOS:31887-31894, a peptide or a fragment of a peptide corresponding to one of the amino acid sequences listed in SEQ ID NOS:31896-31899, a peptide or a fragment of a peptide corresponding to one of the amino acid sequences listed in SEQ ID NOS:31900-31906, a peptide comprising the amino acid sequence IVIAKLKA, a peptide comprising the amino acid sequence IVIAKLKANLMCKTCRLAK, and a peptide or a fragment of a peptide corresponding to one of the amino acid sequences listed in SEQ ID NOS:31907-31922.

62. A cell-penetrating peptide and/or a non-peptide analogue thereof and/or a vector according to any of claims 22-27, 29, 31, 33, 35, 37, 39, 41, 45, 46, 52, 59 and 55-60, further characterised by being cell and/or cell-type and/or tissue specific.

63. A cell-penetrating peptide and/or a non-peptide analogue thereof and/or a vector according to claim 62, wherein said peptide and/or a non-peptide analogue thereof and/or vector selectively interacts with a cell surface protein, thus mediating the cell and/or cell-type and/or tissue specific cellular penetration.

64. A cell-penetrating peptide and/or a non-peptide analogue thereof and/or a vector according to claim 63, wherein said cell surface protein is over-expressed in said specific cell and/or cell-type and/or tissue.

65. A cell-penetrating peptide and/or a non-peptide analogue thereof and/or a vector according to claim 63 or 64, wherein said cell surface protein is selected from the group consisting of receptor tyrosine kinase type receptors, glycosphingolipids, CD44, erbB2, erbB3, and neuropeptide receptors.

66. A cell-penetrating peptide and/or a non-peptide analogue thereof and/or a vector according to claim 62, wherein said peptide and/or vector selectively interacts with an over-expressed cellular and/or extracellular protein, thus mediating the cell and/or cell-type and/or tissue specific cellular penetration.

67. A cell-penetrating peptide and/or a non-peptide analogue thereof and/or a vector according to claim 62, wherein said over-expressed protein is selected from the group consisting of agonists and antagonists to cell and/or cell-type and/or tissue specific receptors.

68. A cell-penetrating peptide and/or a non-peptide analogue thereof and/or a vector according to claim 62 or 63, wherein said over-expressed protein is selected from the group consisting of proteases, protease inhibitors and protease activators.

69. Use of a cell-penetrating peptide and/or a non-peptide analogue thereof and/or a vector according to any of claims 17-68 and/or a cell-selective delivery system according to claim 54 for the manufacture of a pharmaceutical composition.

70. A pharmaceutical composition manufactured according to claim 69.

71. Use of a cell-penetrating peptide and/or a non-peptide analogue thereof and/or a vector and/or a cell-selective delivery system and/or a pharmaceutical composition according to any of claims 17-68 for gene therapy.

72. Use of a cell-penetrating peptide and/or a non-peptide analogue thereof and/or a vector and/or a cell-selective delivery system according to any of claims 17-68 for the manufacture of a pharmaceutical composition for gene therapy.

73. Use of a cell-penetrating peptide and/or a non-peptide analogue thereof and/or a vector and/or a cell-selective delivery system according to any of claims 17-68 for the manufacture of a drug delivery system for transmembrane transport across an epithelial membrane, such as across the epithelium in the intestinal/buccal system, the mucosa in the mouth, lung, rectum or nose, or the blood brain barrier of a mammal.

74. Use of a cell-penetrating peptide and/or a non-peptide analogue thereof and/or a vector, a pharmaceutical composition and/or a drug delivery system according to any of claims 17-68 for the manufacture of a pharmaceutical composition for treating and/or preventing a medical condition selected from the group consisting of infectious diseases, diabetes type I, diabetes type II, Alzheimers Disease, Parkinssons Disease, cancer.

75. Method for treating a patient who suffers from a medical condition, the method comprising administering a pharmaceutical composition comprising a cell-penetrating peptide and/or a non-peptide analogue thereof and/or a vector, a pharmaceutical composition and/or a drug delivery system according to any of claims 17-68 to a patient in need thereof.

76. Method of treating a patient who suffers from a medical condition selected from the group consisting of diabetes type I and II, Alzheimers Disease, Parkinssons Disease, a prion disease, a cardiovascular disease, an infectious disease, disorders resulting from perturbed signal transduction, or cancer, the method comprising administering a pharmaceutical composition comprising a cell-penetrating peptide and/or a non-peptide analogue thereof and/or a vector, a pharmaceutical composition and/or a drug delivery system according to any of claims 17-68 is administered to a patient in need thereof.
